(19) United States
(12) Patent Application Publication
(10) Pub. No.: US 2018/0030118 A1
(43) Pub. Date: Feb. 1, 2018

(54) HUMANZIED FILOVIRUS ANTIBODIES AND USES THEREOF

(71) Applicant: Emergent BioSolutions Canada Inc., Winnipeg (CA)

(72) Inventor: Darrell JOHNSTONE, Winnipeg (CA)

(73) Assignee: Emergent BioSolutions Canada Inc., Winnipeg, MB (CA)

(21) Appl. No.: 15/552,007

(22) PCT Filed: Feb. 19, 2016
(86) PCT No.: PCT/CA2016/000040

§ 371 Date: Aug. 18, 2017

Related U.S. Application Data
(60) Provisional application No. 62/118,432, filed on Feb. 19, 2015.

(51) Int. Cl. C07K 16/10; G01N 33/569

(52) U.S. Cl. CPC C07K 16/10; G01N 33/56983; C07K 2317/565; C07K 2317/567; C07K 2317/56; C07K 2317/622; C07K 2317/55; C07K 2317/54; C07K 2317/624; C07K 2317/24; C07K 2317/76; A61K 2039/505

(57) ABSTRACT

The present disclosure relates to an antibody or antigen-binding portion thereof that binds to a filovirus. The antibody or antigen-binding portion thereof may have one or more murine CDRs and one or more human framework, regions. Also provided herein are compositions comprising the antibody or antigen-binding portion t hereof, methods of producing the antibody or antigen-binding portion thereof and methods of using the antibody or antigen-binding portion thereof.

### CROSS-REFERENCE TO RELATED APPLICATIONS

This application claims priority to U.S. Provisional Application No. 62/118,432, filed Feb. 19, 2015, which is hereby incorporated by reference in its entirety for all purposes.

### FIELD OF THE DISCLOSURE

The present disclosure relates to an antibody or antigen-binding portion thereof that binds to a Filovirus, compositions comprising the antibody or antigen-binding portion thereof, methods of producing the antibody or antigen-binding portion thereof and methods of using the antibody or antigen-binding portion thereof.

### DESCRIPTION OF THE TEXT FILE SUBMITTED ELECTRONICALLY

The content of the text file submitted electronically herewith is incorporated herein by reference in its entirety: A computer readable format copy of the Sequence Listing (filename: EMER_051_01WOSeqList_ST25.txt); date recorded: Feb. 19, 2016; file size 140 KB).

### BACKGROUND

Filovirus infections have been associated with severe fatality rates in humans. Filoviruses are enveloped, negative-strand RNA viruses and can cause lethal hemorrhagic fever in humans and non-human primates. The Filoviridae family includes the genera Marburgvirus and Ebolavirus.

The Marburgvirus genus includes the species, Marburg marburgvivus, which includes the members Marburg virus (MAV) and Ravn virus (RAVV). There are at least two MARV strains, Musoke and Popp, which were identified when the Ravn virus and Angola virus emerged.

The Ebolavirus (EBOV) is a pleiomorphic filamentous virus in the Filoviridae family. Infection with EBOV usually causes a severe hemorrhagic fever, with 50-90% lethality. The outbreak frequency of EBOV has also increased recently. Five different species of EBOV have been identified: Zaire, Sudan, Cote d'Ivoire, Reston and Bundibugyo, each named after the location in which the species was first described. All species are believed to be lethal to humans, with the possible exception of the rare Cote d'Ivoire species and the Reston species. Of these species, the Zaire species of Ebolavirus (ZFBOV) is believed to be the most common and the most lethal.

The negative-stranded RNA genome of a filovirus encodes seven genes. The proteins encoded by the seven genes include the glycoprotein (GP). The GP is responsible for attached and entry of the virus into target cells and is expressed as a precursor that is cleaved to yield two subunits: GP1, which contains the putative receptor-binding region and heavily glycosylated mucin domain: and GP2, which drives membrane fusion.

Natural survival from filovirus infection is rare and not clearly understood. There are currently no approved vaccines or therapeutics for filovirus infection. Development of neutralizing antibodies in the context of natural infection is believed to be difficult. Accordingly, there is a need for the development of vaccines and therapeutics for Filovirus infection. The present disclosure addresses these needs and provides related advantages.

### SUMMARY OF THE DISCLOSURE

The present disclosure provides an antibody or antigen-binding portion thereof that binds to a filovirus. In some embodiments, the antibody or antigen-binding portion thereof that binds to a Filovirus, is an isolated antibody or antigen-binding portion thereof comprising: (a) a light chain CDR1 comprising an amino acid sequence that has at least about 85% sequence identity to an amino acid sequence comprising SEQ NO: 6, 30, 54, 78, 102, 126, 150, 174 or 198; (b) a light chain CDR2 comprising an amino acid sequence that has at least about 85% sequence identity to an amino acid sequence comprising SFQ ID NO: 7, 31, 55, 79, 103, 127, 151, 175, or 199; (c) a light chain CDR3 comprising an amino acid sequence that has at least about 85% sequence identity to an amino acid sequence comprising SEQ ID NO: 8, 32, 56, 80, 104, 128, 152, 176, or 200; (d) a heavy chain CDR1 comprising an amino acid sequence that has at least about 85% sequence identity to an amino acid sequence comprising SEQ ID NO: 18, 42, 66, 90, 114, 138, 162, 186, or 210; (e) a heavy chain CDR2 comprising an amino acid sequence that has at least about 85% sequence identity to an amino acid sequence comprising SEQ ID NO: 19, 43, 67, 91, 115, 139, 163, 187, or 211; (f) a heavy chain CDR3 comprising an amino acid sequence that has at least about 85% sequence identity to an amino acid sequence comprising SEQ ID NO: 20, 44, 68, 92, 116, 140, 164, 188, or 212; (g) a light chain FR1 comprising an amino acid sequence that has at least about 85% sequence identity to an amino acid sequence e comprising SEQ ID NO: 333, 341, 349, 357, 365, 373, 381, 389, or 397; (h) a light chain FR2 comprising an amino acid sequence that has at least about 85% sequence identity to an amino acid sequence comprising SEQ ID NO: 334, 342, 350, 358, 366, 374, 382, 390 or 398; (i) a light chain FR3 comprising an amino acid sequence that has at least about 85% sequence identity to an amino acid sequence comprising SEQ ID NO: 335, 343, 351, 359, 367, 375, 383, 391 or 399; (j) a light chain FR4 comprising an amino acid sequence that has at least about 85% sequence identity to an amino acid sequence comprising SEQ ID NO: 336, 344, 352, 360, 368, 376, 384, 392, or 400; (k) a heavy chain FR1 comprising an amino acid sequence that has at least about 85% sequence identity to an amino acid sequence comprising SEQ ID NOs: 337, 345, 353, 361, 369, 377, 385, 393, or 401; (l) a heavy chain FR2 comprising an amino acid sequence that has at least about 85% sequence identity to an amino acid sequence comprising SEQ ID NO: 338, 346, 354, 362, 370, 378, 386, 394, or 402; (m) a heavy chain FR3 comprising an amino acid sequence that has at least about 85% sequence identity to an amino acid sequence comprising SEQ ID NO: 339, 347, 355, 363, 371, 379, 387, 395 or 403; and, (n) a heavy chain FR4 comprising an amino acid sequence that has at least about 85% sequence identity to an amino acid sequence comprising SEQ ID NO: 340, 348, 356, 364, 372, 380, 388, 396, or 404.

In another embodiment, the isolated antibody or antigen-binding portion thereof comprises: (a) a light chain CDR1, CDR2, and CDR3 comprising an amino acid sequence that has at least about 85% sequence identity to an amino acid sequence comprising SEQ ID NOs: 6, 7 and 8, respectively; a heavy chain CDR1, CDR2, and CDR3 comprising an amino acid sequence that has at least about 85% sequence identity to an amino acid sequence comprising SEQ ID NOs: 18, 19 and 20, respectively; a light chain FR1, FR2, FR3, and FR4 comprising an amino acid sequence that has at least about 85% sequence identity to an amino acid sequence comprising SEQ ID NOs: 333, 334, 335 and 336, respectively; and a heavy chain FR1, FR2, FR3, and FR4 comprising an amino acid sequence that has at least about 85% sequence identity to an amino acid sequence comprising SEQ ID NOs: 337, 338, 339, and 340, respectively; (b) a light chain CDR1, CDR2, and CDR3 comprising an amino acid sequence that has at least about 85% sequence identity to an amino acid sequence comprising SEQ ID NOs: 30, 31, and 32, respectively; a heavy chain CDR1, CDR2, and CDR3 comprising an amino acid sequence that has at least about 85% sequence identity to an amino acid sequence comprising SEQ ID NOs: 42, 43, and 44, respectively; a light chain FR1, FR2, FR3, and FR4 comprising an amino acid sequence that has at least about 85% sequence identity to an amino acid sequence e comprising SEQ ID NOs: 341, 342, 343, and 344, respectively; and a heavy chain FR1, FR2, FR3, and FR4 comprising an amino acid sequence that has at least about 85% sequence identity to an amino acid sequence comprising SEQ ID NOs: 345, 346, 347, and 348, respectively; (c) a light chain CDR1, CDR2, and CDR3 comprising an amino acid sequence that has at least about 85% sequence identity to an amino acid sequence comprising SEQ ID NOs: 54, 55, and 56, respectively; a heavy chain CDR1, CDR2, and CDR3 comprising an amino acid sequence that has at least about 85% sequence identity to an amino acid sequence comprising SEQ ID NOs: 66, 67, and 68, respectively; a light chain FR1, FR2, FR3, and FR4 comprising an amino acid sequence that has at least about 85% sequence identity to an amino acid sequence comprising SEQ ID NOs: 349, 350, 351, and 352, respectively; and a heavy chain FR1, FR2, FR3, and FR4 comprising an amino acid sequence that has at least about 85% sequence identity to an amino acid sequence comprising SEQ ID NOs: 353, 354, 355, and 356, respectively; (d) a light chain CDR1, CDR2, and CDR3 comprising an amino acid sequence that has at least about 85% sequence identity to an amino acid sequence comprising SEQ ID NOs: 78, 79 and 80, respectively; a heavy chain CDR1, CDR2, and CDR3 comprising an amino acid sequence that has at least about 85% sequence identity to an amino acid sequence comprising SEQ ID NOs: 90, 91 and 92, respectively; a light chain FR1, FR2, FR3, and FR4 comprising an amino acid sequence that has at least about 85% sequence identity to an amino acid sequence comprising SEQ ID NOs: 357, 358, 359 and 360, respectively; and a heavy chain FR1, FR2, FR3, and FR4 comprising an amino acid sequence that has at least about 85% sequence identity to an amino acid sequence comprising SEQ NOs: 361, 362, 363, and 364, respectively; (e) a light chain CDR1, CDR2, and CDR3 comprising an amino acid sequence that has at least about 85% sequence identity to an amino acid sequence comprising SEQ ID NOs:102, 103, and 104, respectively; a heavy chain CDR1, CDR2, and CDR3 comprising an amino acid sequence that has at least about 85% sequence identity to an amino acid sequence comprising SEQ ID NOs: 114, 115, and 116, respectively; a light chain FR1, FR2, FR3, and FR4 comprising an amino acid sequence that has at least about 85% sequence identity to an amino acid sequence comprising SEQ ID NOs: 365, 366, 367, and 368, respectively; and a heavy chain FR1, FR2, FR3, and FR4 comprising an amino acid sequence that has at least about 85% sequence identity to an amino acid sequence comprising SEQ ID NOs: 369, 370, 371, and 372, respectively; CD a light chain CDR1, CDR2, and CDR3 comprising an amino acid sequence that has at least about 85% sequence identity to an amino acid sequence comprising SEQ ID NOs: 126, 127, and 128, respectively; a heavy chain CDR1, CDR2, and CDR3 comprising an amino acid sequence that has at least about 85% sequence identity to an amino acid sequence comprising SEQ ID NOs: 138, 139, and 140, respectively; a light chain FR1, FR2, FR3, and FR4 comprising an amino acid sequence that has at least about 85% sequence identity to an amino acid sequence comprising SEQ ID NOs: 373, 374, 375, and 376, respectively; and a heavy chain FR1, FR2, FR3, and FR4 comprising an amino acid sequence that has at least about 85% sequence identity to an amino acid sequence comprising SEQ ID NOs: 377, 378, 379, and 380, respectively; (g) a light chain CDR1, CDR2, and CDR3 comprising an amino acid sequence that has at least about 85% sequence identity to an amino acid sequence comprising SEQ ID NOs: 150, 151., and 152, respectively; a heavy chain CDR1, CDR2, and CDR3 comprising an amino acid sequence that has at least about 85% sequence identity to an amino acid sequence comprising SEQ ID NOs: 162, 163, and 164, respectively; a light chain FR1, FR2, FR3, and FR4 comprising an amino acid sequence that has at least about 85% sequence identity to an amino acid sequence comprising SEQ 381, 382, 383, and 384, respectively; and a heavy chain FR1, FR2, FR3, and FR4 comprising an amino acid sequence that has at least about 85% sequence identity to an amino acid sequence comprising SEQ ID NOs: 385, 386, 387 and 388, respectively; (h) a light chain CDR1, CDR2, and CDR3 comprising an amino acid sequence that has at least about 85% sequence identity to an amino acid sequence comprising SEQ NOs: 174, 175, and 176, respectively; a heavy chain CDR1, CDR2, and CDR3 comprising an amino acid sequence that has at least about 85% sequence identity to an amino acid sequence comprising SEQ ID NOs: 186, 187, and 188, respectively; a light chain FR1, FR2, FR3, and FR4 comprising an amino acid sequence that has at least about 85% sequence identity to an amino acid sequence comprising SEQ NOs: 389, 390, 391, and 392, respectively; and a heavy chain FR1 FR2, FR3, and FR4 comprising an amino acid sequence that has at least about 85% sequence identity to an amino acid sequence comprising SEQ ID NOs: 393, 394, 395, and 396, respectively; or (i) a light chain CDR1, CDR2, and CDR3 comprising an amino acid sequence that has at least about 85% sequence identity to an amino acid sequence comprising SEQ ID NOs: 198, 199 and 200, respectively; a heavy chain CDR1, CDR2, and CDR3 comprising an amino acid sequence that has at least about 85% sequence identity to an amino acid sequence comprising SEQ ID NOs: 210, 211, and 212, respectively; a light chain FR1, FR2, FR3, and FR4 comprising an amino acid sequence that has at least about 85% sequence identity to an amino acid sequence comprising SEQ ID NOs: 397, 398, 399, and 400, respectively; and a heavy chain FR1, FR2, FR3, and FR4 comprising an amino acid sequence that has at least about 85% sequence identity to an amino acid sequence comprising SEQ ID NOs: 401, 402, 403 and 404, respectively.

in some embodiments, the antibody or antigen-binding portion thereof that binds to a filovirus, is an isolated antibody or antigen-binding portion thereof comprising: (a) a light chain CDR1 comprising an amino acid sequence that has at least about 85% sequence identity to an amino acid sequence encoded by a nucleotide sequence comprising SEQ ID NO: 3, 27, 51, 75, 99, 123, 147, 171 or 195; (b) a light chain CDR2 comprising an amino acid sequence that has at least about 85% sequence identity to an amino acid sequence encoded by a nucleotide sequence comprising SEQ ID NO: 4, 28, 52, 76, 100, 124, 148, 172, or 196; (c) a light chain CDR3 comprising an amino acid sequence that has at least about 85% sequence identity to an amino acid sequence encoded by a nucleotide sequence comprising SEQ ID NO: 5, 29, 53, 77, 101, 125, 149, 173, or 197; (d) a heavy chain CDR1 comprising an amino acid sequence that has at least about 85% sequence identity to an amino acid sequence encoded by a nucleotide sequence comprising SEQ ID NO: 15, 39, 63, 87, 111, 135, 159, 183, or 207; (e) a heavy chain CDR2 comprising an amino acid sequence that has at least about 85% sequence identity to an amino acid sequence encoded by a nucleotide sequence comprising SEQ ID NO: 16, 40, 64, 88, 112, 136, 160, 184, or 208; (f) a heavy chain CDR3 comprising an amino acid sequence that has at least about 85% sequence identity to an amino acid sequence encoded by a nucleotide sequence comprising SEQ ID NO: 17, 41, 65, 89, 113, 137, 161, 185 or 209; (g) a light chain FR1 comprising an amino acid sequence that has at least about 85% sequence identity to an amino acid sequence encoded by a nucleotide sequence comprising SEQ ID NO: 9, 33, 57, 81, 105, 129, 153, 177, or 201; (h) a light chain FR2 comprising an amino acid sequence that has at least about 85% sequence identity to an amino acid sequence encoded by a nucleotide sequence comprising SEQ ID NO: 10, 34, 58, 82, 106, 130, 154, 178, or 202; (i) a light chain FR3 comprising an amino acid sequence that has at least about 85% sequence identity to an amino acid sequence encoded by a nucleotide sequence comprising SEQ ID NO: 11, 35, 59, 83, 107 131, 155, 179 or 203; (j) a light chain FR4 comprising an amino acid sequence that has at least: about 85% sequence identity to an amino acid sequence encoded by a nucleotide sequence comprising SEQ ID NO: 12, 36, 60, 84, 108, 132, 156, 180 or 204; (k) a heavy chain FR1 comprising an amino acid sequence that has at least about 85% sequence identity to an amino acid sequence encoded by a nucleotide sequence comprising SEQ ID NO: 21, 45, 69, 93, 117, 141, 165, 189, or 213; (l) a heavy chain FR2 comprising an amino acid sequence that has at least about 85% sequence identity to an amino acid sequence encoded by a nucleotide sequence comprising SEQ ID NO: 22, 46, 70, 94, 118, 142, 166, 190 or 214; (m) a heavy chain FR3 comprising an amino acid sequence that has at least about 85% sequence identity to an amino acid sequence encoded by a nucleotide sequence comprising SEQ ID NO: 23, 47, 71, 95, 119, 143, 167, 191 or 215; and, (n) a heavy chain FR4 comprising an amino acid sequence that has at least about 85% sequence identity to an amino acid sequence encoded by a nucleotide sequence comprising SEQ ID NO: 24, 48, 72, 96, 120, 144, 168, 192 or 216.

In some embodiments, the isolated antibody or antigen-binding portion thereof comprises: (a) a light chain CDR1, CDR2, and CDR3 comprising an amino acid sequence that has at least about 85% sequence identity to an amino acid sequence encoded by a nucleotide sequence comprising SEQ ID NOs: 3, 4, and 5, respectively; a heavy chain CDR1, CDR2, and CDR3 comprising an amino acid sequence that has at least about 85% sequence identity to an amino acid sequence encoded by a nucleotide sequence comprising SEQ ID NOs: 15, 16, and 17, respectively; a light chain FR1, FR2, FR3, and FR4 comprising an amino acid sequence that has at least about 85% sequence identity to an amino acid sequence encoded by a nucleotide sequence comprising SEQ ID NOs: 9, 10, 11, and 12, respectively; and a heavy chain FR1, FR2, FR3, and FR4 comprising an amino acid sequence that has at least about 85% sequence identity to an amino acid sequence encoded by a nucleotide sequence comprising SEQ ID NOs: 21, 22, 23, and 24, respectively; (b) a light chain CDR1, CDR2, and CDR3 comprising an amino acid sequence that has at least about 85% sequence identity to an amino acid sequence encoded by a nucleotide sequence comprising SEQ ID NOs: 27, 28, and 29, respectively; a heavy chain CDR1, CDR2, and CDR3 comprising an amino acid sequence that has at least about 85% sequence identity to an amino acid sequence encoded by a nucleotide sequence comprising SEQ ID NOs: 39, 40, and 41, respectively; a light chain FR1, FR2, FR3, and FR4 comprising an amino acid sequence that has at least about 85% sequence identity to an amino acid sequence encoded by a nucleotide sequence comprising SEQ ID NOs: 33, 34, 35, and 36, respectively; and a heavy chain FR1, FR2, FR3, and FR4 comprising an amino acid sequence that has at least about 85% sequence identity to an amino acid sequence encoded by a nucleotide sequence comprising SEQ ID NOs: 45, 46, 47 and 48, respectively; (c) a light chain CDR1, CDR2, and CDR3 comprising an amino acid sequence that has at least about 85% sequence identity to an amino acid sequence encoded by a nucleotide sequence comprising SEQ ID NOs: 51, 52, and 53, respectively; a heavy chain CDR1, CDR2, and CDR3 comprising an amino acid sequence that has at least about 85% sequence identity to an amino acid sequence encoded by a nucleotide sequence comprising SEQ ID NOs: 63, 64, and 65, respectively: a light chain FR1, FR2, FR3, and FR4 comprising an amino acid sequence that has at least about 85% sequence identity to an amino acid sequence encoded by a nucleotide sequence comprising SEQ ID NOs: 57, 58, 59 and 60, respectively; and a heavy chain FR1, FR2, FR3, and FR4 comprising an amino acid sequence that has at least about 85% sequence identity to an amino acid sequence encoded by a nucleotide sequence comprising SEQ ID NOs: 69, 70, 71, and 72, respectively; (d) a light chain. CDR1, CDR2, and CDR3 comprising an amino acid sequence that has at least about 85% sequence identity to an amino acid sequence encoded by a nucleotide sequence comprising SEQ ID NOs: 75, 76, and 77, respectively; a heavy chain CDR1, CDR2, and CDR3 comprising an amino acid sequence that has at least about 85% sequence identity to an amino acid sequence encoded by a nucleotide sequence comprising SEQ ID NOs: 87, 88, and 89, respectively; a light chain FR1, FR2, FR3, and FR4 comprising an amino acid sequence that has at least about 85% sequence identity to an amino acid sequence encoded by a nucleotide sequence comprising SEQ ID NOs: 81, 82, 83, and 84, respectively; and a heavy chain FR1, FR2, FR3, and FR4 comprising an amino acid sequence that has at least about 85% sequence identity to an amino acid sequence encoded by a nucleotide sequence comprising SEQ ID NOs: 93, 94, 95, and 96, respectively; (e) a light chain CDR1, CDR2, and CDR3 comprising an amino acid sequence that has at least about 85% sequence identity to an amino acid sequence encoded by a nucleotide sequence comprising SEQ ID NOs: 99, 100, and 101, respectively; a heavy chain CDR1, CDR2, and CDR3 comprising an amino acid sequence that has at least about 85% sequence identity to an amino acid sequence encoded by a nucleotide sequence comprising SEQ ID NOs: 111, 112, and 113, respectively; a light chain FR1, FR2, FR3, and FR4 comprising an amino acid sequence that has at least about 85% sequence identity to an amino acid sequence encoded by a nucleotide sequence comprising SEQ ID NOs: 105, 106, 107, and 108, respectively; and a heavy chain FR1, FR2, FR3, and FR4 comprising an amino acid sequence that has at least about 85% sequence identity to an amino acid sequence encoded by a nucleotide sequence comprising SEQ ID NOs: 117, 118, 119, and 120, respectively; (f) a light chain CDR1, CDR2, and CDR3 comprising an amino acid sequence that has at least about 85% sequence identity to an amino acid sequence encoded by a nucleotide sequence comprising SEQ ID NOs: 123, 124 and 125, respectively; a heavy chain CDR1, CDR2, and CDR3 comprising an amino acid sequence that has at least about 85% sequence identity to an amino acid sequence encoded by a nucleotide sequence comprising SEQ ID NOs: 135, 136, and 137, respectively; a light chain FR1, FR2, FR3, and FR4 comprising an amino acid sequence that has at least about 85% sequence identity to an amino acid sequence encoded by a nucleotide sequence comprising SEQ ID NOs: 129, 130, 131, 132, respectively; and a heavy chain FR1, FR2, FR3, and FR4 comprising an amino acid sequence that has at least about 85% sequence identity to an amino acid sequence encoded by a nucleotide sequence comprising SEQ ID NOs: 141, 142, 143, and 144, respectively; (g) a light chain CDR1, CDR2, and CDR3 comprising an amino acid sequence that has at least about 85% sequence identity to an amino acid sequence encoded by a nucleotide sequence comprising SEQ ID NOs: 147, 148 and 149, respectively; a heavy chain CDR1, CDR2, and CDR3 comprising an amino acid sequence that has at least about 85% sequence identity to an amino acid sequence encoded by a nucleotide sequence comprising SEQ ID NOs: 159, 160, and 161, respectively; a light chain FR1, FR2, FR3, and FR4 comprising an amino acid sequence that has at least about 85% sequence identity to an amino acid sequence encoded by a nucleotide sequence comprising SEQ ID NOs: 153, 154, 155, and 156, respectively; and a heavy chain FIR1, FR2, FR3, and FR4 comprising an amino acid sequence that has at least about: 85% sequence identity to an amino acid sequence encoded by a nucleotide sequence comprising SEQ ID NOs: 165, 166, 167, and 168, respectively; (h) a light chain CDR1, CDR2, and CDR3 comprising an amino acid sequence that: has at least about 85% sequence identity to an amino acid sequence encoded by a nucleotide sequence comprising SEQ ID NOs: 171, 172, and 173, respectively; a heavy chain CDR1, CDR2, and CDR3 comprising an amino acid sequence that has at least about 85% sequence identity to an amino acid sequence encoded by a nucleotide sequence comprising SEQ ID NOs: 183, 184, and 185, respectively; a light chain FR1, FR2, FR3, and FR4 comprising an amino acid sequence that has at least about 85% sequence identity to an amino acid sequence encoded by a nucleotide sequence comprising SEQ ID NOs: 177, 178, 179 and 180, respectively; and a heavy chain FR1, FR2, FR3, and FR4 comprising an amino acid sequence that has at least about 85% sequence identity to an amino acid sequence encoded by a nucleotide sequence comprising SEQ ID NOs: 189, 190, 191, and 192, respectively; or (i) light chain CDR1, CDR2, and CDR3 comprising an amino acid sequence that has at least about 85% sequence identity to an amino acid sequence encoded by a nucleotide sequence comprising SEQ ID NOs: 195, 196 and 197, respectively; a heavy chain CDR1, CDR2, and CDR3 comprising an amino acid sequence that has at least about 85% sequence identity to an amino acid sequence encoded by a nucleotide sequence comprising SEQ ID NOs: 207, 208, and 209, respectively; a light chain FR1, FR2, FR3, and FR4 comprising an amino acid sequence that has at least about 85% sequence identity to an amino acid sequence encoded by a nucleotide sequence comprising SEQ ID NOs: 201, 202, 203 and 204. respectively; and a heavy chain FR1, FR2, FR3, and FR4 comprising an amino acid sequence that has at least about 85% sequence identity to an amino acid sequence encoded by a nucleotide sequence comprising SEQ D NOs: 213, 214, 215 and 216, respectively.

In some embodiments, the antibody or antigen-binding portion thereof comprises: (a) a variable heavy chain comprising an amino acid sequence that has at least about 98% sequence identity to an amino acid sequence encoded by a nucleotide sequence comprising SEQ ID NO: 13, 37, 61, 85, 109, 133, 157, 181, or 205; and (b) a variable light chain comprising an amino acid sequence that has at least about 98% sequence identity to an amino acid sequence encoded by a nucleotide sequence comprising SEQ ID NO: 1, 25, 49, 73, 97, 121, 145, 169, or 193. In some embodiments, the antibody or antigen-binding portion thereof comprises: (a) a variable heavy chain comprising an amino acid sequence that has at least about 98% sequence identity to an amino acid sequence encoded by a nucleotide sequence comprising SEQ ID NO: 13 and a variable light chain comprising an amino acid sequence that has at least about 98% sequence identity to an amino acid sequence encoded by a nucleotide sequence comprising SEQ ID NO: 1; (b) a variable heavy chain comprising an amino acid sequence that has at least about 98% sequence identity to an amino acid sequence encoded by a nucleotide sequence comprising SEQ ID NO: 37 and a variable light chain comprising an amino acid sequence that has at least about 98% sequence identity to an amino acid sequence encoded by a nucleotide sequence comprising SEQ ID NO: 25; (c) a variable heavy chain comprising an amino acid sequence that has at least about 98% sequence identity to an amino acid sequence encoded by a nucleotide sequence comprising SEQ ID NO: 61 and a variable light chain comprising an amino acid sequence that has at least about 98% sequence identity to an amino acid sequence encoded by a nucleotide sequence comprising SEQ LD NO: 49; (d) a variable heavy chain comprising an amino acid sequence that has at least about 98% sequence identity to an amino acid sequence encoded by a nucleotide sequence comprising SEQ ID NO: 85 and a variable light chain comprising an amino acid sequence that has at least about 98% sequence identity to an amino acid sequence encoded by a nucleotide sequence comprising SEQ ID NO: 73; (e) a variable heavy chain comprising an amino acid sequence that has at least about 98% sequence identity to an amino acid sequence encoded by a nucleotide sequence comprising SEQ ID NO: 109 and a variable light chain comprising an amino acid sequence that has at least about 98% sequence identity to an amino acid sequence encoded by a nucleotide sequence comprising SEQ ID NO: 97; (f) a variable heavy chain comprising an amino acid sequence that has at least about 98% sequence identity to an amino acid sequence encoded by a nucleotide sequence comprising SEQ ID NO: 133 and a variable light chain comprising an amino acid sequence that has at least about 98% sequence identity to an amino acid sequence encoded by a nucleotide sequence comprising SEQ ID NO: 121; (g) a variable heavy chain comprising an amino acid sequence that has at least about 98% sequence identity to an amino acid sequence encoded by a nucleotide sequence comprising SEQ ID NO: 157 and a variable light chain comprising an amino acid sequence that has at least about 98% sequence identity to an amino acid sequence encoded by a nucleotide sequence comprising SEQ ID NO: 145; (h) a variable heavy chain comprising an amino acid sequence that has at least about 98% sequence identity to an amino acid sequence encoded by a nucleotide sequence comprising SEQ ID NO: 181 and a variable light chain comprising an amino acid sequence that has at least about 98% sequence identity to an amino acid sequence encoded by a nucleotide sequence comprising SEQ ID NO: 169; or (i) a variable heavy chain comprising an amino acid sequence that has at least about 98% sequence identity to an amino acid sequence encoded by a nucleotide sequence comprising SEQ ID NO: 205 and a variable light chain comprising an amino acid sequence that has at least about 98% sequence identity to an amino acid sequence encoded by a nucleotide sequence comprising SEQ ID NO: 193.

In some embodiments, the isolated antibody or antigen-binding portion thereof that binds to a filovirus comprises: (a) a variable heavy chain comprising an amino acid sequence that has at least about 98% sequence identity to SEQ ID NO: 14, 38, 62, 86, 110, 134, 158, 182, or 206; and (b) a variable light chain comprising an amino acid sequence that has at least about: 98% sequence identity to SEQ ID NO: 2, 26, 50, 74, 98, 122, 146, 170 or 194. In some embodiments, the isolated antibody or antigen-binding portion thereof comprises: (a) a variable heavy chain comprising an amino acid sequence that has at least about 98% sequence identity to SEQ ID NO: 14 and a variable light chain comprising an amino acid sequence that has at least about 98% sequence identity to SEQ ID NO: 2; (b) a variable heavy chain comprising an amino acid sequence that has at least about 98% sequence identity to SEQ ID NO: 38 and a variable light chain comprising an amino acid sequence that has at least about 98% sequence identity to SEQ ID NO: 26; (c) a variable heavy chain comprising an amino acid sequence that has at least about 98% sequence identity to SEQ ID NO: 62 and a variable light chain comprising an amino acid sequence that has at least about 98% sequence identity to SEQ ID NO: 50; (d) a variable heavy chain comprising an amino acid sequence that has at least about 98% sequence identity to SEQ ID NO: 86 and a variable light chain comprising an amino acid sequence that has at least about 98% sequence identity to SEQ ID NO: 74; (e) a variable heavy chain comprising an amino acid sequence that has at least about 98% sequence identity to SEQ ID NO: 110 and a variable light chain comprising an amino acid sequence that has at least about 98% sequence identity to SEQ ID NO: 98; (f) a variable heavy chain comprising an amino acid sequence that has at least about 98% sequence identity to SEQ ID NO: 134 and a variable light chain comprising an amino acid sequence that has at least about 98% sequence identity to SEQ ID NO: 122; (g) a variable heavy chain comprising an amino acid sequence that has at least: about 98% sequence identity to SEQ ID NO: 158 and a variable light chain comprising an amino acid sequence that has at least about 98% sequence identity to SEQ ID NO: 146; (h) a variable heavy chain comprising an amino acid sequence that has at least: about 98% sequence identity to SEQ ID NO: 182 and a variable light chain comprising an amino acid sequence that has at least about 98% sequence identity to SEQ ID NO: 170; or (i) a variable heavy chain comprising an amino acid sequence that has at least about 98% sequence identity to SEQ ID NO: 206 and a variable light chain comprising an amino acid sequence that has at least about 98% sequence identity to SEQ ID NO: 194.

In yet other embodiments, the isolated antibody or antigen-binding portion thereof comprises: (a) a variable heavy chain comprising an amino acid sequence comprising SEQ ID NO: 14 and a variable light chain comprising an amino acid sequence comprising SEQ ID NO: 2; (b) a variable heavy chain comprising an amino acid sequence comprising SEQ ID NO: 38 and a variable light chain comprising an amino acid sequence comprising SEQ ID NO: 26; (c) a variable heavy chain comprising an amino acid sequence comprising SEQ ID NO: 62 and a variable light chain comprising an amino acid sequence comprising SEQ ID NO: 50; (d) a variable heavy chain comprising an amino acid sequence comprising SEQ ID NO: 86 and a variable light chain comprising an amino acid sequence comprising SEQ NO: 74; (e) a variable heavy chain comprising an amino acid sequence comprising SEQ ID NO: 110 and a variable light chain comprising an amino acid sequence comprising SEQ ID NO: 98; (I) a variable heavy chain comprising an amino acid sequence comprising SEQ ID NO: 134 and a variable light chain comprising an amino acid sequence comprising SEQ ID NO: 122; (g) a variable heavy chain comprising an amino acid sequence comprising SEQ ID NO: 158 and a variable light chain comprising an amino acid sequence comprising SEQ ID NO: 146; (h) a variable heavy chain comprising an amino acid sequence comprising SEQ ID NO: 182 and a variable light chain comprising an amino acid sequence comprising SEQ NO: 170; or (i) a variable heavy chain comprising an amino acid sequence comprising SEQ ID NO: 206 and a variable light chain comprising an amino acid sequence comprising SEQ ID NO: 194.

The isolated antibody or antigen-binding portion thereof disclosed herein can be a whole immunoglobulin, an scFv, a Fab fragment, an F(ab′)2, or a disulfide linked Fv.

In some embodiments, the isolated antibody of antigen-binding portion thereof binds to the GP subunit of a filovirus, e.g., to a GP1 or GP2 subunit. In some embodiments, the antibody or antigen-binding portion thereof binds to the mucin domain of the GP1 subunit or wing domain of the GP2 subunit. The filovirus can be Ebolavirus or Marburgvirus, such as Zaire ebolavirus, Sudan ebolavirus, Reston ebolavirus, Tai Forest ebolavirus, Cote d'Ivoire ebolavirus, Bundibugyo ebolavirus, Marburg virus or Ravn virus, In some embodiments, the antibody is cross-reactive to at least two filoviruses.

Also provided herein is a nucleic acid sequence encoding an antibody or antigen-binding portion thereof disclosed herein. The present disclosure also provides an expression vector comprising a promoter operably linked to a nucleotide sequence disclosed herein, such as a nucleic acid sequence encoding an antibody or antigen-binding portion thereof disclosed herein. In some embodiments, the expression vector comprises a nucleotide sequence with the following sequences: (a) SEQ ID NOs: 3, 4, 5, 9, 10, 11, 12, 15, 16, 17, 21, 22, 23, and 24; (b) SEQ ID NOs: 27, 28, 29, 33, 34, 35, 36, 39, 40, 41, 45, 46, 47, and 48; (c) SEQ ID NOs: 51, 52, 53, 57, 58, 59, 60, 63, 64, 65, 69, 70, 71, and 72; (d) SEQ ID NOs: 75, 76, 77, 81, 82, 83, 84, 87, 88, 89, 93, 94, 95, and 96; (e) SEQ ID NOs: 99, 100, 101, 105, 106, 107, 108, 111, 112, 113, 117, 118, 119, and 120, (f) SEQ ID NOs: 123, 124, 125, 129, 130, 131, 132, 135, 136, 137, 141, 142, 143, and 144; (g) SEQ ID NOs: 147, 148, 149, 153, 154, 155, 156, 159, 160, 161, 165, 166, 167, and 168; (h) SEQ ID NOs: 171, 172, 173, 177, 178, 179, 180, 183, 184, 185, 189, 190, 191, and 192; or (i) SEQ ID NOs: 195, 196, 197, 201, 202, 203, 204, 207, 208, 209, 213, 214, 215 and 216. In some embodiments, the expression vector comprises a nucleotide sequence selected from the group consisting of SEQ ID NOs. 1, 13, 25, 37, 49, 61, 73, 85, 97, 109, 121, 133, 145, 157, 169, 181, 193, or 205. In some embodiments, the expression vector comprises a nucleotide sequence according to SEQ ID NO: 1, 25, 49, 73, 97, 121, 145, 169, or 193. In some embodiments, the expression vector comprises a nucleotide sequence according to SEQ ID NO: 13, 37, 61, 85, 109, 133, 157, 181, or 205. In other embodiments, the expression vector comprises a nucleotide sequence with the following sequences: (a) SEQ ID NOs: 1 and 13; (b) SEQ ID NOs: 25 and 37; (c) SEQ ID NOs: 49 and 61; (d) SEQ ID NOs: 73 and 85; (e) SEQ ID NOs: 97 and 109; (1) SEQ ID NOs: 121 and 133; (g) SEQ ID NOs: 145 and 157; (h) SEQ ID NOs: 169 and 181; or (i) SEQ ID NOs: 193 and 205.

Also provided herein is a host cell comprising an expression vector disclosed herein. In some embodiments, the cell is a bacterial, eukaryotic or mammalian cell. The cell can he a COS-1, COS-7, HEK293, BHK21, CHO, BSC-1, HepG2, SP210, HeLa, myeloma or lymphoma cell.

The present disclosure also provides a method of producing an antibody or antigen-binding portion thereof that binds to a filovirus disclosed herein. In some embodiments, the method comprises culturing a host cell, such as one disclosed herein, and recovering the antibody or antigen-binding portion thereof.

The present disclosure also provides compositions comprising an antibody or antigen-binding portion thereof disclosed herein. In some embodiments, the composition is a pharmaceutical composition comprising an antibody or antigen-binding portion thereof. The pharmaceutical composition can further comprise a pharmaceutically acceptable carrier.

In some embodiments, the composition comprises an antibody or antigen-binding portion thereof disclosed herein and one or more other antibodies or antigen-binding portions thereof. In some embodiments, the one or more other antibodies or antigen-binding portions thereof can bind a protein produced by a virus in the Filoviridae family. In some embodiments, the protein is a glycoprotein. The virus can be Ebolavirus or Marburgvirus. In some embodiments, the virus is Zaire ebolavirus, Sudan ebolavirus, Reston ebolavirus, Tai Forest ebolcivirus, Cote d'Ivoire ebolavirus, Bundibugyo ebolavirus, Marburg virus or Ravn virus. The composition can further comprise a pharmaceutically acceptable carrier.

Also provided herein are methods comprising administering an antibody or antigen-binding portion disclosed herein. In one embodiment, a method for reducing, treating or preventing a filovirus infection (e.g., a Marburgvirus or Ebolavirus infection) in a subject in need thereof comprising administering to the subject a therapeutically effective amount of the antibody or antigen-binding portion thereof disclosed herein is provided. In some embodiments, a method for reducing, treating or preventing an filovirus infection (e.g., a Marburgvirus or Ebolavirus infection) in a subject in need thereof comprises administering to the subject a therapeutically effective amount of a composition disclosed herein. In some embodiments, the subject is a human.

Methods for detecting a filovirus (e.g., Marburgvirus or Ebolavirus) in a sample is also provided herein. In some embodiments, the method comprises contacting the sample with an antibody or antigen-binding portion thereof disclosed herein. In some embodiments, the sample is a cell, tissue, or biological fluid from a subject suspected of having or at risk of a filovirus infection.

### DETAILED DESCRIPTION

The present disclosure relates to an antibody or antigen-binding portion thereof that binds to a filovirus, compositions comprising the antibody or antigen-binding portion thereof, methods of producing the antibody or antigen-binding portion thereof and methods of using the antibody or antigen-binding portion thereof, which are described in further detail below.

The section headings used herein arc for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls however, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means ±20% of the indicated range, value, or structure, unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

As used herein, “an antibody that binds to a filovirus” is used interchangeably with “an anti-filovirus antibody,” An anti-filovirus antibody refers to a monoclonal or recombinant antibody or antibody fragment that binds to a filovirus with specificity. The antibody can be human, humanized or chimeric. An anti-filovirus antibody or antigen-binding portion thereof includes any antibody or substance having a binding domain with the required specificity. Thus, an anti-filovirus antibody or antigen-binding portion thereof includes antibody fragments, derivatives, functional equivalents and homologues of antibodies, humanized antibodies, including any polypeptide comprising an immunoglobulin binding domain, whether natural or wholly or partially synthetic. Chimeric molecules comprising an immunoglobulin binding domain, or equivalent, fused to another polypeptide are also included. A humanized antibody may be a modified antibody having the variable regions of a non-human, e.g., murine, antibody and the constant region of a human antibody. A humanized antibody may also be a modified antibody having the variable regions of a non-human, e.g., marine, antibody and the framework region(s) of a human antibody. An anti-filovirus antibody includes anti-Ebolavirus antibodies and anti-Marburgvirus antibodies, in which an anti-Ebolavirus antibody refers to a monoclonal or recombinant antibody or antibody fragment that binds to an Ebolavirus with specificity and an anti-Marburgvirus antibody refers to a monoclonal or recombinant antibody or antibody fragment that binds to a with Marburgvirus specificity. An anti-filovirus antibody also includes an antibody that is cross-reactive to an Ebolavirus and a Marburgvirus.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. If derived from a non-human source, other regions of the antibody or immunoglobulin binding proteins and polypeptides, such as the hinge region and constant region domains, can also be humanized.

The terms “light chain variable region” (also referred to as “light chain variable domain” or “VL” or V_{L}) and “heavy chain variable region” (also referred to as “heavy chain variable domain” or “VH” or V_{H}) refer to the variable binding region from an antibody light and heavy chain, respectively. The variable binding regions are made up of discrete, well-defined sub-regions known as “complementarity determining regions” (CDRs) and “framework regions” (FRs). In one embodiment, the FRs are humanized The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH3, and CH4 domains (IgE, IgM). A. “Fab” (fragment antigen binding) is the part of an antibody that binds to antigens and includes the variable region and CH1 domain of the heavy chain linked to the light chain via an inter-chain disulfide bond.

The six “complementarity determining regions” or “CDRs” present in an antibody antigen-binding domain are short, non-contiguous sequences of amino acids that are specifically positioned to form the binding domain as the antibody assumes its three dimensional configuration in an aqueous environment. The remainder of the amino acids in the binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, cart be readily identified for any given heavy or light chain variable region by one of ordinary skill in the art, since they have been defined in various different ways (see, “Sequences of Proteins of Immunological interest,” Kabat, E., et al., U.S. Department of Health and Human Services, (1983); and Chothia and Lesk J. Mol. Biol., 196:901-917 (1987), which are incorporated herein by reference in their entireties). In some embodiments, an antibody, or antigen-binding fragment thereof, contains at least one heavy chain variable region and/or at least one light chain variable region. The heavy chain variable region (or light chain variable region) typically contains three CDRs and four framework regions (FRs), arranged from amino-terminus to carboxyl-terminus in the following order: FR1, CDR1, FR2, CDR2, FR3, CDR3, FR4.

In the case where there are two or more definitions of a term which is used and/or accepted within the art, the definition of the term as used herein is intended to include all such meanings unless explicitly stated to the contrary. A specific example is the use of the tern “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

CDRs can also be determined using IMGT® (the international ImMunoGeneTics information system®) numbering. H: heavy chain; K: kappa or L: light chain. Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

As used herein, the term “antigen-binding portion,” “antigen-binding region” “or antigen-binding domain” refers to the domain, region, portion, or site of a protein, polypeptide, oligopeptide, or peptide or antibody or binding domain derived from an antibody that possesses the ability to specifically recognize and bind to an antigen. Exemplary binding antigen-binding portions include single-chain antibody variable regions (e.g., domain antibodies, sFv, scFv, scFab). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis.

An antibody or antigen-binding portion “specifically binds” an antigen if it binds the antigen with an affinity or Kₐ (i.e., an equilibrium association constant of a particular binding interaction with units of 1/M) equal to or greater than 10⁵ M⁻¹, while not significantly binding other components present in a test sample. The antibody or antigen-binding portion can be classified as “high affinity” or “low affinity.” “High affinity” refer to those antibodies or antigen-binding portions with a Kₐ of at least about 10⁷ M⁻¹, at least about 10⁸ M⁻, at least about 10⁹ M⁻¹, at least about 10¹⁰ M⁻¹, at least about 10¹¹ M⁻¹, at least about 10¹² M⁻¹, or at least about 10¹³ M⁻¹. “Low affinity” r refer to those antibodies or antigen-binding portions with a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁵ M to 10⁻¹³ M). Affinities of refer to those antibodies or antigen-binding portions according to the present: disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

As used herein “reference antibody” refers to an antibody that is known in the art and which serves the basis for a humanized, chimeric or recombinant antibody. The reference antibody may be a non-human, (e.g., murine), human, humanized, chimeric and/or recombinant antibody or antibody-like polypeptide.

¢Treatment” or “treating” refers to either a therapeutic treatment or prophylactic/preventative treatment. A therapeutic treatment may improve at least one symptom of disease in an individual receiving treatment or may delay worsening of a progressive disease in an individual, or prevent onset of additional associated diseases.

Ameliorating or reducing or reduction of infection), as used herein, can include but is not limited to delaying the onset of the infection, attenuating the symptoms of the infection, shortening the duration of the infection, reducing the viral titer in a patient (e.g., in the blood), or slowing the progression of the infection. Filovirus infections encompassed by the present application include, but are not limited to, Marburgvirus and Ebolavirus.

A “therapeutically effective amount,” “therapeutically effective dose” or “effective dose” refers to that amount of the antibody or compound sufficient to result in amelioration of one or more symptoms of the disease being treated. When applied to an individual active ingredient, administered alone, a therapeutically effective dose refers to that ingredient alone. When applied to a combination, a therapeutically effective dose refers to combined amounts of the active ingredients that result in the therapeutic effect, whether administered serially or simultaneously. One or more specific therapeutic molecules may be administered according to methods of the invention, each in an effective close. The effective dose can be determined empirically through dose studies. The term “therapeutically effective amount” is used interchangeably with “prophylactically effective amount” herein, and refers to an amount that prevents infection with a filovirus, prevents disease associated with a filovirus infection, reduces the number and/or severity of symptoms of a filovirus infection, stops or limits the spread of a filovirus, and/or shortens the duration of a filovirus infection.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia fir use in animals, and more particularly in humans are considered to be “pharmaceutically acceptable.”

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule.” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least about: 20, 30, 40, 50, 60, 70. 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least about 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least about 80%, at least about 85%, at least about 90%, at least about 95%, at least about 96%, at least about 97%, at least about 98%, or at least about 99% sequence identity relative to each other.

The present disclosure relates to an antibody or antigen-binding portion thereof that binds to a filovirus, compositions comprising the antibody or antigen-binding portion thereof, methods of producing the antibody or antigen-binding portion thereof and methods of using the antibody or antigen-binding portion thereof.

The terms “antibody that binds to a filovirus” and “anti-filovirus antibody” are used interchangeably herein. Examples of an anti-filovirus antibody or antigen-binding portion thereof, include, but are not limited to, (i) the Fab fragment consisting of VL, VH, CL and CH domains; (ii) the Fd fragment consisting of the VH and CH domains; (iii) the Fv fragment consisting of the VL and VH domains of a single antibody (e.g., linked by a disulfide bond); (iv) the dAb fragment (Ward, E. S. et al., Nature 341: 544-546 (1989)) which consists of a VH domain; (v) isolated CDR regions; (vi) F(ab′)₂ a bivalent fragment comprising two linked Fab fragments; (vii) single chain Fv molecules (scFv), wherein a VH domain and a VL domain are linked by a peptide linker which allows the two domains to associate to form an antigen binding site (Bird et al., Science 242: 423-426 (1988); Huston et al., PNAS USA 85: 5879-5883 (1988)); (viii) bispecific single chain Fv dimers (PCT/US92/09965); (ix) “diabodics”, multivalent or multispecific fragments constructed by gene fusion (WO94/13804; P. Hollinger et al., Proc. Natl. Acad. Sci. USA 90: 6444-6448 (1993)); and (x) whole immunoglobulin.

In one embodiment, an “anti-filovirus antibody” is a recombinant anti-filovirus antibody or polypeptide. Recombinant anti-filovirus antibodies and polypeptides include, for instance, Fc fusions, toxin fusions, fusions to enzymatic activities, minibodies, diabodies, linear antibodies, single chain antibodies, bispecific antibody fragments, say and Fab fragments. A recombinant anti-filovirus antibody includes a molecule or polypeptide that incorporates an amino acid sequence derived from an anti-filovirus antibody and which is capable of binding a filovirus with specificity. Recombinant anti-filovirus antibodies include molecules that are optimized, for instance, for stability, solubility, in vitro and in vivo binding.

In one embodiment, an anti-filovirus antibody is a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associated with each other to form an antigen binding site: antigen binding sites are formed by the association of the first domain of one polypeptide within the multimer with the second domain of another polypeptide within the mailmen. See WO94/13804 which is incorporated by reference in its entirety.

In one embodiment, an anti-Filovirus antibody is a scFv. A scFv is constructed by joining a variable heavy chain and a variable light chain with a linker using recombinant methods. The linker that enables the V_{H} and V_{L} regions to be made as a single chain protein. See, for instance, Bird et al., 1988, Science 242:423-426 and Huston et al., 1988, Proc. Natl. Acad. Sci. USA 85:5879-5883. In one embodiment, the scFv comprises V_{H} and V_{L} regions that are identical or derived from a reference anti-filovirus antibody.

In one embodiment, an anti-filovirus antibody or antigen-binding portion thereof is an Fv. An Fv is an antibody fragment which contains a complete antigen-recognition and binding site. This region consists of a dimer alone heavy and one light chain variable domain in tight, non-covalent or covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. In one embodiment, the Fv comprises V_{H} and V_{L} regions that are identical or derived from a reference anti-filovirus antibody.

In one embodiment of the invention, an anti-filovirus antibody is a single chain polypeptide comprising, from amino to carboxyl terminus, a binding domain (e.g., scFv), an immunoglobulin hinge region and an immunoglobulin constant region. In this embodiment, also known as a small modular immunopharmaecutical (SMIP), the single chain polypeptide forms a dimer in solution.

The anti-filovirus antibody or antigen-binding portion thereof can be a recombinant polypeptide, fusion protein or immunoconjugate that binds a filovirus and comprise an antibody fragment or are derived in part from a monoclonal or polyclonal anti-filovirus antibody. In some embodiments, an anti-filovirus antibody or antigen-binding portion thereof is a molecule or polypeptide that is derived from a reference anti-filovirus antibody and is capable of binding with specificity to the same epitope as the reference anti-filovirus antibody. In some embodiments, the epitope is on a GP subunit of a filovirus, e.g., a GP1 or GP2 subunit. Thus, in one embodiment, the anti-filovirus antibody or antigen-binding portion thereof binds to the GP1 or GP2 subunit of the filovirus. In some embodiments, the anti-filovirus antibody or antigen-binding portion thereof binds to the mucin domain of the GP1 subunit or the wing domain of the GP2 subunit. The GP subunit can be from Ebolavirus or Marburgvirus such as Zaire ebolavirus, Sudan ebolavirus, Reston ebolavirus, Tai Forest ebolavirus, Cote d'Ivoire ebolavirus, Bundibugyo ebolavirus, Marburg virus or Ravn virus.

In some embodiments, the anti-filovirus antibody binds to one or more specifies of Ebolavirus. In some embodiments, the anti-filovirus antibody binds to Marburg virus or Ravn virus. In some embodiments, the anti-filovirus antibody binds to at least two filoviruses. In some embodiments, the anti-filovirus antibody binds to an Ebolavirus or Marburgvirus.

An anti-filovirus derived from a reference anti-filovirus antibody can include a molecule or polypeptide comprising at least about 10 contiguous amino acids, at least about 20 contiguous amino acids or at least about 50 or more contiguous amino acids as the reference anti-filovirus antibody.

In one embodiment, an anti-filovirus antibody or antigen-binding portion thereof comprises a heavy chain CDR1, heavy chain CDR2, heavy chain CDR3, light chain CDR1, light chain CDR2, and/or light chain CDR3 with the same amino acid sequence as a reference anti-filovirus antibody. In another embodiment, an anti-filovirus antibody or antigen-binding portion thereof comprises a heavy chain CDR1, heavy chain CDR2, heavy chain CDR3, light chain CDR1, light chain CDR2, and/or light chain CDR3 that has an amino acid sequence comprising a sequence that has at least about 85%, 86%, 87%, 88%, 88%, 89%, 90%, 91%, 92%, 93%, 94%, 95%, 96%, 97%, 98%, or 99% sequence identity to an amino acid sequence of the respective heavy chain CDR1, heavy chain CDR2, heavy chain CDR3, light chain CDR1, light chain CDR2, and/or light chain CDR3 of a reference anti-filovirus antibody or molecule.

In another embodiment, an anti-filovirus antibody or antigen-binding portion thereof comprises a VH and/or VL with the same amino acid sequence as a reference anti-filovirus molecule. In another embodiment, an anti-filovirus antibody or antigen-binding portion thereof comprises a VH and/or VL that has an amino acid sequence comprising a sequence that has at least about 85%, 86%, 87%, 88%, 88%, 89%, 90%, 91%, 92%, 93%, 94%, 95%, 96%, 97%, 98%, or 99% sequence identity to an amino acid sequence of the respective VH andior VL of a reference anti-filovirus molecule.

In some embodiments, the reference anti-Filovirus molecule is a murine anti-filovirus antibody. In some embodiments, the reference anti-filovirus molecule is a murine anti-Marburgvirus antibody. In one embodiment, the reference anti-filovirus molecule is the murine anti-Filovirus antibody CAN30G5, CAN54G2, CAN30G2, CAN40G1, CAN30G1, CAN30G3, or CAN30G4. The CDRs, VHs and VLs of these murine antibodies are provided in Table 2.

TABLE 2

| | Chain, | | | SEQ ID |
|---|---|---|---|---|
| Name | Region | Origin | Sequence | NO: |
| huCAN30G5 | K, | Artificial | gacatcgtgctgacccagtcccccctgtccctgcccgtgaccctgggcca | 1 |
| | Variable | sequence | gcccgcctccatctcctgccgctcctcccagtccctggtgcactccaacg | |
| | region | | gcaacacctacctccactggtaccagcagcgccccggccagtcccccc | |
| | | | gcctgagatctacaaggtgtccaaccgcttctccggcgtgcccgaccgc | |
| | | | ttctccggctccggctccggcaccgacttcaccctgaagatctcccgcgt | |
| | | | ggaggccgaggacgtgggcgtgtactactgctcccagtccacccacgtg | |
| | | | ccctggaccttcggcggcggcaccaaggtggagatcaagc | |
| huCAN30G5 | K, | Artificial | DIVLTQSPLSLPVTLGQPASISCRSSQSLVHSNG | 2 |
| | variable | sequence | NTYLHWYQQRPGQSPRLLIYKVSNRFSGVPDR | |
| | region | | FSGSGSGTDFTLKISRVEAEDVGVYYCSQSTHV | |
| | | | PWTFGGTKVEIK | |
| huCAN30G5 | K, CDR1 | Artificial | cagtccctggtgcactccaacggcaacacctac | 3 |
| | | sequence | | |
| huCAN30G5 | K, CDR2 | Artificial | aaggtgtcc | 4 |
| | | sequence | | |
| huCAN30G5 | K, CDR3 | Artificial | tcccagtccacccacgtgccctggacc | 5 |
| | | sequence | | |
| huCAN30G5 | K, CDR1 | Artificial | QSLVHSNGNTY | 6 |
| | | sequence | | |
| huCAN30G5 | K, CDR2 | Artificial | KVS | 7 |
| | | sequence | | |
| huCAN30G5 | K, CDR3 | Artificial | SQSTHVPWT | 8 |
| | | sequence | | |
| huCAN30G5 | K, FR1 | Artificial | gacatcgtgagacccagtcccccctgtccctgcccgtgaccctgggcca | 9 |
| | | sequence | gcccgcctccatctcctgccgctcctcc | |
| huCAN30G5 | K, FR2 | Artificial | ctgcactggtaccagcagcgccccggccagtccccccgcctgctgatct | 10 |
| | | sequence | ac | |
| huCAN30G5 | K, FR3 | Artificial | aaccgcttctccggcgtgcccgaccgcttctccggctccggctccggca | 11 |
| | | sequence | ccgacttcaccctgaagatctcccgcgtggaggccgaggacgtgggcgt | |
| | | | gtactactgc | |
| huCAN30G5 | K, FR4 | Artificial | ttcggcggcggcaccaaggtggagatcaagc | 12 |
| | | sequence | | |
| huCAN30G5 | H, | Artificial | gaggtgcagctggtggagtccggcggcggcctggtgcagcccggcgg | 13 |
| | Variable | sequence | ctccctgaagctgtcctgcgccgcctccggcttcgccttcaactcctacgc | |
| | region | | catgcactgggtgcgccaggcctccggcaagggcctggagtgggtggc | |
| | | | ccgcatccgcatcaagtccggcaactacgccacctcctacgccggctcc | |
| | | | gtgaagggccgcttcaccgtgtcccgcgacgactccaagaacaccttcta | |
| | | | cctgcagatgaactccctgaagaccgaggacaccgccatgtactactgc | |
| | | | gtgcgcgagtgggagggcgccatggactactggggccagggcaccct | |
| | | | ggtgaccgtgtcctccg | |
| huCAN30G5 | H, | Artificial | EVQLVESGGGLVQPGGSLKLSCAASGFAFNSY | 14 |
| | variable | sequence | AMHWVRQASGKGLEWVARIRIKSCINYATSYA | |
| | region | | GSVKGRFTVSRDDSKNTFYLQMNSLKTEDTA | |
| | | | MYYCVREWEGAMDYWGQGTLVTVSS | |
| huCAN30G5 | H, CDR1 | Artificial | ggcttcgccttcaactcctacgcc | 15 |
| | | sequence | | |
| huCAN30G5 | H, CDR2 | Artificial | atccgcatcaagtccggcaactacgccacc | 16 |
| | | sequence | | |
| huCAN30G5 | H, CDR3 | Artificial | gtgcgcgagtgggagggcgccatggactac | 17 |
| | | sequence | | |
| huCAN30G5 | H, CDR1 | Artificial | GFAFNSYA | 18 |
| | | sequence | | |
| huCAN30G5 | H, CDR2 | Artificial | IRIKSGNYAT | 19 |
| | | sequence | | |
| huCAN30G5 | H, CDR3 | Artificial | VREWEGAMDY | 20 |
| | | sequence | | |
| huCAN30G5 | H, FR1 | Artificial | gaggtgcagctggtggagtccggcggcggcctggtgcagcccggctt | 21 |
| | | sequence | ctccctgaagctgtcctgcgccgcctcc | |
| huCAN30G5 | H, FR2 | Artificial | atgcactgggtgcgccaggcctccggcaagggcctggagtgggtggcc | 22 |
| | | sequence | cgc | |
| huCAN30G5 | H, FR3 | Artificial | tcctacgccggctccgtgaagggccgcttcaccgtgtcccgcgacgact | 23 |
| | | sequence | ccaagaacaccttctacctgcagatgaactccctgaagaccgaggacac | |
| | | | cgccatgtactactgc | |
| huCAN30G5 | H, FR4 | Artificial | tggggccagggcaccctggtgaccgtgtcctccg | 24 |
| | | sequence | | |
| rehuCAN30G5 | K, | Artificial | gacatcgtgctgacccagtcccccctgtccctgcccgtgaccctgggcca | 25 |
| | Variable | sequence | gcccgcctccatctcctgccgctcctcccagtccctggtgcactccaacg | |
| | region | | gcaacacctacctgcactggtacctgcagaagcccggccagtccccccg | |
| | | | cctgctgatctacaaggtgtccaaccgcttctccggcgtgcccgaccgctt | |
| | | | ctccggctccggctccggcaccgacttcaccctgaagatctcccgcgtg | |
| | | | gaggccgaggacgtgggcgtgtacttctgctcccagtccacccacgtgc | |
| | | | cctggaccttcggcggcggcacaagctggagatcaag | |
| rehuCAN30G5 | K, | Artificial | DIVLTQSPLSLPVTLGQPASISCRSSQSLVIISING | 26 |
| | variable | sequence | NTYLHWYLQKPGQSPRLLIYKVSNRFSGVPDR | |
| | region | | FSGSGSGTDFTLKISRVEAEDVGVYFCSQSTHV | |
| | | | PWTFGGGTKLEIK | |
| rehuCAN30G5 | K, CDR1 | Artificial | cagtccctggtgcactccaacggcaacacctac | 27 |
| | | sequence | | |
| rehuCAN30G5 | K, CDR2 | Artificial | aaggtgtcc | 28 |
| | | sequence | | |
| rehuCAN30G5 | K, CDR3 | Artificial | tcccagtccacccacgtgccctggacc | 29 |
| | | sequence | | |
| rehuCAN30G5 | K, CDR1 | Artificial | QSLVHSNGNTY | 30 |
| | | sequence | | |
| rehuCAN30G5 | K, CDR2 | Artificial | KVS | 31 |
| | | sequence | | |
| rehuCAN30G5 | K, CDR3 | Artificial | SQSTHVPWT | 32 |
| | | sequence | | |
| rehuCAN30G5 | K, FR1 | Artificial | gacatcgtgctgacccagtcccccctgtccctgcccgtgaccctgggcca | 33 |
| | | sequence | gcccgcctccatctcctgccgctcctcc | |
| rehuCAN30G5 | K, FR2 | Artificial | ictgcactggtacctgcagaagcccggccagtccccccgcctgctgatcta | 34 |
| | | sequence | c | |
| rehuCAN30G5 | K, FR3 | Artificial | aaccgcttctccggcgtgcccgaccgcttctccggctccggctccggca | 35 |
| | | sequence | ccgacttcaccctgaagatctcccgcgtggaggccgaggacgtgggcgt | |
| | | gtacttctgc | | |
| rehuCAN30G5 | K, FR4 | Artificial | ttcggcggcggcaccaagctggagatcaag | 36 |
| | | sequence | | |
| rehuCAN30G5 | H, | Artificial | gaggtgcagctggtggagtccggcggcggcctggtgcagcccggcgg | 37 |
| | Variable | sequence | ctccctgaagctgtcctgcgccgcctccggcttcgccttcaactcctacgc | |
| | region | | catgcactgggtgtgccaggcctccggcaagggcctggagtgggtggc | |
| | | | ccgcatccgcatcaagtccggcaactacgccacctcctacgccggctcc | |
| | | | gtgaagggccgcttcaccgtgtcccgcgacgactccaagtccctgttcta | |
| | | | cctgcagatgaacaacctgaagaccgaggacaccgccatgtactactgc | |
| | | | gtgcgcgagtgggagggcgccatggactactggggccagggcaccct | |
| | | | ggtgaccgtgtcctcc | |
| rehuCAN30G5 | H, | Artificial | EVQLVESGGGLVQPGGSLKLSCAASGFAFNSY | 38 |
| | variable | sequence | AMHWVCQASGKGLEWVARIRIKSGNYATSYA | |
| | region | | GSVKGRFTVSRDDSKSLFYLQMNNLKTEDTA | |
| | | | MYYCVREWEGAMDYWGQGTLVTVSS | |
| rehuCAN30G5 | H, CDR1 | Artificial | ggcttcgccttcaactcctacgcc | 39 |
| | | sequence | | |
| rehuCAN30G5 | H, CDR2 | Artificial | atccgcatcaagtccggcaactacgccacc | 40 |
| | | sequence | | |
| rehuCAN30G5 | H, CDR3 | Artificial | gtgcgcgagtgggagggcgccatggactac | 41 |
| | | sequence | | |
| rehuCAN30G5 | H, CDR1 | Artificial | GFAFNSYA | 42 |
| | | sequence | | |
| rehuCAN30G5 | H, CDR2 | Artificial | IRIKSGNYAT | 43 |
| | | sequence | | |
| rehuCAN30G5 | H, CDR3 | Artificial | VREWEGAMDY | 44 |
| | | sequence | | |
| rehuCAN30G5 | H, FR1 | Artificial | gaggtgcagctggtggagtccggcggcggcctggtgcagcccggcgg | 45 |
| | | sequence | ctccctgaagctgtcctgcgccgcctcc | |
| rehuCAN30G5 | H, FR2 | Artificial | atgcactgggtgtgccaggcctccggcaagggcctggagtgggtggcc | 46 |
| | | sequence | cgc | |
| rehuCAN30G5 | H, FR3 | Artificial | tcctacgccggctccgtgaagggccgcttcaccgtgtcccgcgacgact | 47 |
| | | sequence | ccaagtccctgttctacctgcagatgaacaacctgaagaccgaggacac | |
| | | | cgccatgtactactgc | |
| rehuCAN30G5 | H, FR4 | Artificial | tggggccagggcaccctggtgaccgtgtcctcc | 48 |
| | | sequence | | |
| cdrCAN30G5 | K, | Artificial | gacgtggtgatgacccagtcccccctgtccctgcccgtgaccctgggcc | 49 |
| | Variable | sequence | agcccgcctccatacctgccgctcctcccagtccctggtgcactccaac | |
| | region | | ggcaacacctacctgaactggttccagcagcgccccggccagtcccccc | |
| | | | gccgcctgatctacaaggtgtccaaccgcgactccggcgtgcccgaccg | |
| | | | cttctccggctccggctccggcaccgacttcaccctgaagatctcccgcg | |
| | | | tggaggccgaggacgtgggcgtgtactactgctcccagtccacccacgt | |
| | | | gccctggaccttcggcggcggcaccaaggtggagatcaag | |
| cdrCAN30G5 | K, | Artificial | DVVMTQSPLSLPVTLGQPASISCRSSQSLVHSN | 50 |
| | variable | sequence | GNTYLNWFQQRPGQSPRRLIYKVSNRDSGVPD | |
| | region | | RFSGSGSGTDFTLKISRVEAEDVGVYYCSQSTH | |
| | | | VPWTFGGGTKVEIK | |
| cdrCAN30G5 | K, CDR1 | Artificial | cagtccctggtgcactccaacggcaacacctac | 51 |
| | | sequence | | |
| cdrCAN30G5 | K, CDR2 | Artificial | aaggtgtcc | 52 |
| | | sequence | | |
| cdrCAN30G5 | K, CDR3 | Artificial | tcccagtccacccacgtgccctggacc | 53 |
| | | sequence | | |
| cdrCAN30G5 | K, CDR1 | Artificial | QSLVHSNGNTY | 54 |
| | | sequence | | |
| cdrCAN30G5 | K, CDR2 | Artificial | KVS | 55 |
| | | sequence | | |
| cdrCAN30G5 | K, CDR3 | Artificial | SQSTHVPWT | 56 |
| | | sequence | | |
| cdrCAN30G5 | K, FR1 | Artificial | gacgtggtgatgacccagtcccccctgtccctgcccgtgaccctgggcc | 57 |
| | | sequence | agcccgcctccatctcctgccgctcctcc | |
| cdrCAN30G5 | K, FR2 | Artificial | ctgaactggttccagcagcgccccggccagtccccccgccgcctgatct | 58 |
| | | sequence | ac | |
| cdrCAN30G5 | K, FR3 | Artificial | aaccgcgactccggcgtgcccgaccgcttctccggctccggctccggca | 59 |
| | | sequence | ccgacttcaccctgaagatctcccgcgtggaggccgaggacgtgggcgt | |
| | | | gtactactgc | |
| cdrCAN30G5 | K, FR4 | Artificial | ttcggcggcggcaccaaggtggagatcaag | 60 |
| | | sequence | | |
| cdrCAN30G5 | H, | Artificial | gaggtgcagctggtggagtccggcggcggcctggtgcagcccggcgg | 61 |
| | Variable | sequence | ctccctgaagctgtcctgcgccgcctccggcttcgccttcaactcctacgc | |
| | region | | catgcactgggtgcgccaggcctccggcaagggcctggagtgggtggg | |
| | | | ccgcatccgcatcaagtccggcaactacgccaccgcctacgccgcctcc | |
| | | | gtgaagggccgcttcaccatctcccgcgacgactccaagaacaccgcct | |
| | | | acctgcagatgaactccctgaagaccgaggacaccgccgtgtactactg | |
| | | | cgtgcgcgagtgggagggcgccatggactactggggccagggcaccc | |
| | | | tggtgaccgtgtcctcc | |
| cdrCAN30G5 | H, | Artificial | EVQLVESGGGLVQPGGSLKLSCAASGFAFNSY | 62 |
| | variable | sequence | AMHWVRQASGKGLEWVGRIRIKSGNYATAYA | |
| | region | | ASVKGRFTISRDDSKNTAYLQMNSLKTEDTAV | |
| | | | YYCVREWEGAMDYWGQGTLVTVSS | |
| cdrCAN30G5 | H, CDR1 | Artificial | ggcttcgccttcaactcctacgcc | 63 |
| | | sequence | | |
| cdrCAN30G5 | H, CDR2 | Artificial | atccgcatcaagtccggcaactacgccacc | 64 |
| | | sequence | | |
| cdrCAN30G5 | H, CDR3 | Artificial | gtgcgcgagtgggagggcgccatggactac | 65 |
| | | sequence | | |
| cdrCAN30G5 | H, CDR1 | Artificial | GFAFNSYA | 66 |
| | | sequence | | |
| cdrCAN30G5 | H, CDR2 | Artificial | IRIKSGNYAT | 67 |
| | | sequence | | |
| cdrCAN30G5 | H, CDR3 | Artificial | VREWEGAMDY | 68 |
| | | sequence | | |
| cdrCAN30G5 | H, FR1 | Artificial | gaggtgcagctggtggagtccggcggcggcctggtgcagcccggcgg | 69 |
| | | sequence | ctccctgaagctgtcctgcgccgcctcc | |
| cdrCAN30G5 | H, FR2 | Artificial | atgcactgggtgcgccaggcctccggcaagggcctggagtgggtgggc | 70 |
| | | sequence | cgc | |
| cdrCAN30G5 | H, FR3 | Artificial | gcctacgccgcctccgtgaagggccgcttcaccatctcccgcgacgact | 71 |
| | | sequence | ccaagaacaccgcctacctgcagatgaactccctgaagaccgaggaca | |
| | | | ccgccgtgtactactgc | |
| cdrCAN30G5 | H, FR4 | Artificial | tggggccagggcaccctggtgaccgtgtcctcc | 72 |
| | | sequence | | |
| huCAN40G1 | K, | Artificial | gacatcgtgctgacccagtcccccgcctccctggccgtgtccctgggcg | 73 |
| | Variable | sequence | agcgcgccaccatctcctgcaaggcctcccagtccgtggaccacgacg | |
| | region | | gcgactcctacatgaactggtaccagcagaagcccggccagcccccca | |
| | | | agctgctgatctacgccacctccaacctggagtccggcatccccgcccg | |
| | | | cttctccggctccggctccggcaccgacttcaccctgaccatctcctccct | |
| | | | gcaggccgaggacgtggccacctactactgccagcagtcctacgaggt | |
| | | | gcccctgaccttcggcgccggcaccaagctggagatcaagc | |
| huCAN40G1 | K, | Artificial | DIVLTQSPASLAVSLGERATISCKASQSVDHDG | 74 |
| | variable | sequence | DSYMNWYQQKPGQPPKLLIYATSNLESGIPAR | |
| | region | | FSGSGSGTDFTLTISSLQAEDVATYYCQQSYEV | |
| | | | PLTFGAGTKLEIK | |
| huCAN40G1 | K, CDR1 | Artificial | cagtccgtggaccacgacggcgactcctac | 75 |
| | | sequence | | |
| huCAN40G1 | K, CDR2 | Artificial | gccacctcc | 76 |
| | | sequence | | |
| huCAN40G1 | K, CDR3 | Artificial | cagcagtcctacgaggtgcccctgacc | 77 |
| | | sequence | | |
| huCAN40G1 | K, CDR1 | Artificial | QSVDHDGDSY | 78 |
| | | sequence | | |
| huCAN40G1 | K, CDR2 | Artificial | ATS | 79 |
| | | sequence | | |
| huCAN40G1 | K, CDR3 | Artificial | QQSYEVPLT | 80 |
| | | sequence | | |
| huCAN40G1 | K, FR1 | Artificial | gacatcgtgctgacccagtcccccgcctccctggccgtgtccctgggcg | 81 |
| | | sequence | agcgcgccaccatctcctgcaaggcctcc | |
| huCAN40G1 | K, FR2 | Artificial | atgaactggtaccagcagaagcccggccagccccccaagctgctgatct | 82 |
| | | sequence | ac | |
| huCAN40G1 | K, FR3 | Artificial | aacctggagtccggcatccccgcccgcttctccggctccggctccggca | 83 |
| | | sequence | ccgacttcaccctgaccatctcctccctgcaggccgaggacgtggccac | |
| | | | ctactactgc | |
| huCAN40G1 | K, FR4 | Artificial | ttcggcgccggcaccaagctggagatcaagc | 84 |
| | | sequence | | |
| huCAN40G1 | H, | Artificial | gaggtgcagctgcagcagtccggccccgaggtgaagaagcccggcgc | 85 |
| | Variable | sequence | ctccgtgaaggtgtcctgccgcacctccggctacaccttcaccgagtaca | |
| | region | | ccatccactgggtgaagcaggcccccggcaagggcctggagtggatcg | |
| | | | gcggcatcaaccccaaccacggcggcaccctgtacaaccagaagttca | |
| | | | agggccgcgtgaccctgaccgtggacaagtcctcctccaccgcctacat | |
| | | | ggagctgtcccgcctgcgctccgacgacaccgccgtgtactactgcgcc | |
| | | | cgcttcacctacgactactggggccagggcaccctggtgaccgtgtcctc | |
| | | | cg | |
| huCAN40G1 | H, | Artificial | EVQLQQSGPEVKKPGASVKVSCRTSGYTFTEY | 86 |
| | variable | sequence | THIWVKQAPGKGLEWIGGINPNHGGTLYNQKF | |
| | region | | KGRVTLTVDKSSSTAYMELSRLRSDDTAVYYC | |
| | | | ARFTYDYWGQGTLVTVSS | |
| huCAN40G1 | H, CDR1 | Artificial | ggctacaccttcaccgagtacacc | 87 |
| | | sequence | | |
| huCAN40G1 | H, CDR2 | Artificial | atcaaccccaaccacggcggcacc | 88 |
| | | sequence | | |
| huCAN40G1 | H, CDR3 | Artificial | gcccgcttcacctacgactac | 89 |
| | | sequence | | |
| huCAN40G1 | H, CDR1 | Artificial | GYTFTEYT | 90 |
| | | sequence | | |
| huCAN40G1 | H, CDR2 | Artificial | INPNHGGT | 91 |
| | | sequence | | |
| huCAN40G1 | H, CDR3 | Artificial | ARFTYDY | 92 |
| | | sequence | | |
| huCAN40G1 | H, FR1 | Artificial | gaggtgcagctgcagcagtccggccccgaggtgaagaagcccggcgc | 93 |
| | | sequence | ctccgtgaaggtgtcctgccgcacctcc | |
| huCAN40G1 | H, FR2 | Artificial | atccactgggtgaagcaggcccccggcaagggcctggagtggatcggc | 94 |
| | | sequence | ggc | |
| huCAN40G1 | H, FR3 | Artificial | ctgtacaaccagaagttcaagggccgcgtgaccctgaccgtggacaagt | 95 |
| | | sequence | cctcctccaccgcctacatggagctgtcccgcctgcgctccgacgacac | |
| | | | cgccgtgtactactgc | |
| huCAN40G1 | H, FR4 | Artificial | tggggccagggcaccctggtgaccgtgtcctccg | 96 |
| | | sequence | | |
| huCAN40G1 | K, | Artificial | gacatcgtgctgacccagtcccccgcctccctggccgtgtccctgggcg | 97 |
| | Variable | sequence | agcgcgccaccatctcctgcaaggcctcccagtccgtggaccacgacg | |
| | region | | gcgactcctacatgaactggtaccagcagaagcccggccagcccccca | |
| | | | agctgctgatctacgccacctccaacctggagtccggcatccccgcccg | |
| | | | cttctccggctccggctccggcaccgacttcaccctgaacatctcctccgt | |
| | | | gcaggccgaggacgtggccacctactactgccagcagtcctacgaggt | |
| | | | gcccctgaccttcggcgccggcaccaagctggagctgaag | |
| rehuCAN40G1 | K, | Artificial | DIVLTQSPASLAVSLGERATISCKASQSVDHDG | 98 |
| | variable | sequence | DSYMNWYQQKPGQPPKLLIYATSNLESGIPAR | |
| | region | | FSGSGSGTDFTLNISSVQAEDVATYYCQQSYEV | |
| | | | PLTFGAGTKLELK | |
| rehuCAN40G1 | K, CDR1 | Artificial | cagtccgtggaccacgacggcgactcctac | 99 |
| | | sequence | | |
| rehuCAN40G1 | K, CDR2 | Artificial | gccacctcc | 100 |
| | | sequence | | |
| rehuCAN40G1 | K, CDR3 | Artificial | cagcagtcctacgaggtgcccctgacc | 101 |
| | | sequence | | |
| rehuCAN40G1 | K, CDR1 | Artificial | QSVDHDGDSY | 102 |
| | | sequence | | |
| rehuCAN40G1 | K, CDR2 | Artificial | ATS | 103 |
| | | sequence | | |
| rehuCAN40G1 | K, CDR3 | Artificial | QQSYEVPLT | 104 |
| | | sequence | | |
| rehuCAN40G1 | K, FR1 | Artificial | gacatcgtgctgacccagtcccccgcctccctggccgtgtccctgggcg | 105 |
| | | sequence | agcgcgccaccatctcctgcaaggcctcc | |
| rehuCAN40G1 | K, FR2 | Artificial | atgaactggtaccagcagaagcccggccagccccccaagctgctgatct | 106 |
| | | sequence | ac | |
| rehuCAN40G1 | K, FR3 | Artificial | aacctggagtccggcatccccgcccgcttctccggctccggctccggca | 107 |
| | | sequence | ccgacttcaccctgaacatctcctccgtgcaggccgaggacgtggccac | |
| | | | ctactactgc | |
| rehuCAN40G1 | K, FR4 | Artificial | ttcggcgccggcaccaagctggagctgaag | 108 |
| | | sequence | | |
| rehuCAN40G1 | H, | Artificial | gaggtgcagctgcagcagtccggccccgaggtggtgaagcccggcgc | 109 |
| | Variable | sequence | ctccgtgaagatctcctgccgcacctccggctacaccttcaccgagtaca | |
| | region | | ccatccactgggtgaagcaggcccccggcaagggcctggagtggatcg | |
| | | | gcggcatcaaccccaaccacggcggcaccctgtacaaccagaagttca | |
| | | | agggccgcgccaccctgaccgtggacaagtcctcctccaccgcctacat | |
| | | | ggagctgtcccgcctgcgctccgacgacaccgccgtgtactactgcgcc | |
| | | | cgcttcacctacgactactggggccagggcaccctgctgaccgtgtcctc | |
| | | | c | |
| rehuCAN40G1 | H, | Artificial | EVQLQQSGPEVVKPGASVKISCRTSGYTFTEYT | 110 |
| | variable | sequence | HIWVKQAPGKGLEWIGGINPNHGGTLYNQKF | |
| | region | | KGRATLTVDKSSSTAYMELSRLRSDDTAVYYC | |
| | | | ARFTYDYWGQGTLLTVSS | |
| rehuCAN40G1 | H, CDR1 | Artificial | ggctacaccttcaccgagtacacc | 111 |
| | | sequence | | |
| rehuCAN40G1 | H, CDR2 | Artificial | atcaaccccaaccacggcggcacc | 112 |
| | | sequence | | |
| rehuCAN40G1 | H, CDR3 | Artificial | gcccgcttcacctacgactac | 113 |
| | | sequence | | |
| rehuCAN40G1 | H, CDR1 | Artificial | GYTFTEYT | 114 |
| | | sequence | | |
| rehuCAN40G1 | H, CDR2 | Artificial | INPNHGGT | 115 |
| | | sequence | | |
| rehuCAN40G1 | H, CDR3 | Artificial | ARFTYDY | 116 |
| | | sequence | | |
| rehuCAN40G1 | H, FR1 | Artificial | gaggtgcagctgcagcagtccggccccgaggtggtgaagcccggcgc | 117 |
| | | sequence | ctccgtgaagatctcctgccgcacctcc | |
| rehuCAN40G1 | H, FR2 | Artificial | atccactgggtgaagcaggcccccggcaagggcctggagtggatcggc | 118 |
| | | sequence | ggc | |
| rehuCAN40G1 | H, FR3 | Artificial | ctgtacaaccagaagttcaagggccgcgccaccctgaccgtggacaagt | 119 |
| | | sequence | cctcctccaccgcctacatggagctgtcccgcctgcgctccgacgacac | |
| | | | cgccgtgtactactgc | |
| rehuCAN40G1 | H, FR4 | Artificial | tggggccagggcaccctgctgaccgtgtcctcc | 120 |
| | | sequence | | |
| cdrCAN40G1 | K, | Artificial | gacatcgtgatgacccagtcccccgactccctggccgtgtccctgggcg | 121 |
| | Variable | sequence | agcgcgccaccatcaactgcaagtcctcccagtccgtggaccacgacg | |
| | region | | gcgactcctacctggcctggtaccagcagaagcccggccagcccccca | |
| | | | agctgctgatctacgccacctccacccgcgagtccggcgtgcccgaccg | |
| | | | cttctccggctccggctccggcaccgacttcaccctgaccatctcctccct | |
| | | | gcaggccgaggacgtggccgtgtactactgccagcagtcctacgaggt | |
| | | | gcccctgaccttcggccagggcaccaagctggagatcaag | |
| cdrCAN40G1 | K, | Artificial | DIVMTQSPDSLAVSLGERATICKSSQSVDHDG | 122 |
| | variable | sequence | DSYLAWYQQKPGQPPKLLIYATSTRESGVPDR | |
| | region | | FSGSGSGTDFTLTISSLQAEDVAVYYCQQSYEV | |
| | | | PLTFGQGTKLEIK | |
| cdrCAN40G1 | K, CDR1 | Artificial | cagtccgtggaccacgacggcgactcctac | 123 |
| | | sequence | | |
| cdrCAN40G1 | K, CDR2 | Artificial | gccacctcc | 124 |
| | | sequence | | |
| cdrCAN40G1 | K, CDR3 | Artificial | cagcagtcctacgaggtgcccctgacc | 125 |
| | | sequence | | |
| cdrCAN40G1 | K, CDR1 | Artificial | QSVDHDGDSY | 126 |
| | | sequence | | |
| cdrCAN40G1 | K, CDR2 | Artificial | ATS | 127 |
| | | sequence | | |
| cdrCAN40G1 | K, CDR3 | Artificial | QQSYEVPLT | 128 |
| | | sequence | | |
| cdrCAN40G1 | K, FR1 | Artificial | gacatcgtgatgacccagtcccccgactccctggccgtgtccctgggcg | 129 |
| | | sequence | agcgcgccaccatcaactgcaagtcctcc | |
| cdrCAN40G1 | K, FR2 | Artificial | ctggcctggtaccagcagaagcccggccagccccccaagctgctgatct | 130 |
| | | sequence | ac | |
| cdrCAN40G1 | K, FR3 | Artificial | acccgcgagtccggcgtgcccgaccgcttctccggctccggctccggc | 131 |
| | | sequence | accgacttcaccctgaccatctcctccctgcaggccgaggacgtggccgt | |
| | | | gtactactgc | |
| cdrCAN40G1 | K, FR4 | Artificial | ttcggccagggcaccaagctggagatcaag | 132 |
| | | sequence | | |
| cdrCAN40G1 | H, | Artificial | caggtgcagctggtgcagtccggcgccgaggtgaagaagcccggcgc | 133 |
| | Variable | sequence | ctccgtgaaggtgtcctgcaaggcctccggctacaccttcaccgagtaca | |
| | region | | ccatgcactgggtgcgccaggcccccggccagggcctggagtggatg | |
| | | | ggctggatcaaccccaaccacggcggcaccaactacgcccagaagttc | |
| | | | cagggccgcgtgaccatgacccgcgacacctccatctccaccgcctaca | |
| | | | tggagctgtcccgcctgcgctccgacgacaccgccgtgtactactgcgc | |
| | | | ccgcttcacctacgactactggggccagggcaccctggtgaccgtgtcct | |
| | | | cc | |
| cdrCAN40G1 | H, | Artificial | QVQLVQSGAEVKKPGASVKVSCKASGYTFTE | 134 |
| | variable | sequence | YTMHWVRQAPGQGLEWMGWINPNHGGTNY | |
| | region | | AQKFQGRVTMTRDTSISTAYMELSRLRSDDTA | |
| | | | VYYCARFTYDYWGQGTLVTVSS | |
| cdrCAN40G1 | H, CDR1 | Artificial | ggctacaccttcaccgagtacacc | 135 |
| | | sequence | | |
| cdrCAN40G1 | H, CDR2 | Artificial | atcaaccccaaccacggcggcacc | 136 |
| | | sequence | | |
| cdrCAN40G1 | H, CDR3 | Artificial | gcccgcttcacctacgactac | 137 |
| | | sequence | | |
| cdrCAN40G1 | H, CDR1 | Artificial | GYTFTEYT | 138 |
| | | sequence | | |
| cdrCAN40G1 | H, CDR2 | Artificial | INPNHGGT | 139 |
| | | sequence | | |
| cdrCAN40G1 | H, CDR3 | Artificial | ARFTYDY | 140 |
| | | sequence | | |
| cdrCAN40G1 | H, FR1 | Artificial | caggtgcagctggtgcagtccggcgccgaggtgaagaagcccggcgc | 141 |
| | | sequence | ctccgtgaaggtgtcctgcaaggcctcc | |
| cdrCAN40G1 | H, FR2 | Artificial | atgcactgggtgcgccaggcccccggccagggcctggagtggatggg | 142 |
| | | sequence | ctgg | |
| cdrCAN40G1 | H, FR3 | Artificial | aactacgcccagaagttccagggccgcgtgaccatgacccgcgacacc | 143 |
| | | sequence | tccatctccaccgcctacatggagctgtcccgcctgcgctccgacgacac | |
| | | | cgccgtgtactactgc | |
| cdrCAN40G1 | H, FR4 | Artificial | tggggccagggcaccctggtgaccgtgtcctcc | 144 |
| | | sequence | | |
| huCAN54G2 | K, | Artificial | gacgtggtgatgacccagacccccctgaccctgcccgtgaccctgggcc | 145 |
| | Variable | sequence | agcccgcctccatctcctgcacctcctcccagtccctgctgaactccgac | |
| | region | | ggcgagacctacctgaactggctgctgcagcgccccggccagtccccc | |
| | | | aagcgcctgatccacctggtgtccaagctggactccggcgtgcccgacc | |
| | | | gcttctccggctccggctccggcaccgacttcaccctgaagatctcccgc | |
| | | | gtggaggccgaggacgtgggcgtgactactgctggcagggcacccac | |
| | | | ttcccctggaccttcggcggcggcaccaaggtggagatcaagc | |
| huCAN54G2 | K, | Artificial | DVVMTQTPLTLPVTLGQPASISCTSSQSLLNSD | 146 |
| | variable | sequence | GETYLNWLLQRPGQSPKRLHILVSKLDSGVPD | |
| | region | | RFSGSGSGTDFTLKISRVEAEDVGVYYCWQGT | |
| | | | HFPWTFGGGTKVEIK | |
| huCAN54G2 | K, CDR1 | Artificial | cagtccctgctgaactccgacggcgagacctac | 147 |
| | | sequence | | |
| huCAN54G2 | K, CDR2 | Artificial | ctggtgtcc | 148 |
| | | sequence | | |
| huCAN54G2 | K, CDR3 | Artificial | tggcagggcacccacttcccctggacc | 149 |
| | | sequence | | |
| huCAN54G2 | K, CDR1 | Artificial | QSLLNSDGETY | 150 |
| | | sequence | | |
| huCAN54G2 | K, CDR2 | Artificial | LVS | 151 |
| | | sequence | | |
| huCAN54G2 | K, CDR3 | Artificial | WQGTHFPWT | 152 |
| | | sequence | | |
| huCAN54G2 | K, FR1 | Artificial | gacgtggtgatgacccagacccccctgaccctgcccgtgaccctgggcc | 153 |
| | | sequence | agcccgcctccatctcctgcacctcctcc | |
| huCAN54G2 | K, FR2 | Artificial | ctgaactggctgctgcagcgccccggccagtcccccaagcgcctgatc | 154 |
| | | sequence | ac | |
| huCAN54G2 | K, FR3 | Artificial | aagctggactccggcgtgcccgaccgcttctccggctccggctccggca | 155 |
| | | sequence | ccgacttcaccctgaagatctcccgcgtggaggccgaggacgtgggcgt | |
| | | | gtactactgc | |
| huCAN54G2 | K, FR4 | Artificial | ttcggcggcggcaccaaggtggagatcaagc | 156 |
| | | sequence | | |
| huCAN54G2 | H, | Artificial | caggtgcagctgcagcagtccggcaccgaggtgaagaagcccggcgc | 157 |
| | Variable | sequence | ctccgtgaaggtgtcctgcaaggcctccggctacgacttcaccaacttctg | |
| | region | | gctgggctggatccgccaggcccccggccagggcctggagtggatcg | |
| | | | gcgacatctaccccggcggcgacaacacctactacaacgagaagttcaa | |
| | | | gggccgcgtgaccctgaccgccgacaagtcctccaacaccgcctacat | |
| | | | ggagctgtcctccctgcgctccgaggacaccgccgtgtacttctgcttcat | |
| | | | gatcctgtacaccctggactactggggccagggcaccctggtgaccgtg | |
| | | | tcctccg | |
| huCAN54G2 | H, | Artificial | QVQLQQSGTEVKKPGASVKVSCKASGYDFTN | 158 |
| | variable | sequence | FWLGWIRQAPGQGLEWIGDIYPGGDNTYYNE | |
| | region | | KFKGRVTLTADKSSNTAYMELSSLRSEDTAVY | |
| | | | FCFMILYTLDYWGQGTLVTVSS | |
| huCAN54G2 | H, CDR1 | Artificial | ggctacgacttcaccaacttctgg | 159 |
| | | sequence | | |
| huCAN54G2 | H, CDR2 | Artificial | atctaccccggcggcgacaacacc | 160 |
| | | sequence | | |
| huCAN54G2 | H, CDR3 | Artificial | ttcatgatcctgtacacctggactac | 161 |
| | | sequence | | |
| huCAN54G2 | H, CDR1 | Artificial | GYDFTNFW | 162 |
| | | sequence | | |
| huCAN54G2 | H, CDR2 | Artificial | IYPGGDNT | 163 |
| | | sequence | | |
| huCAN54G2 | H, CDR3 | Artificial | FMILTYLDY | 164 |
| | | sequence | | |
| huCAN54G2 | H, FR1 | Artificial | caggtgcagctgcagcagtccggcaccgaggtgaagaagcccggcgc | 165 |
| | | sequence | ctccgtgaaggtgtcctgcaaggcctcc | |
| huCAN54G2 | H, FR2 | Artificial | ctgggctggatccgccaggcccccggccagggcctggagtggatcgg | 166 |
| | | sequence | cgac | |
| huCAN54G2 | H, FR3 | Artificial | tactacaacgagaagttcaagggccgcgtgaccctgaccgccgacaagt | 167 |
| | | sequence | cctccaacaccgcctacatggagctgtcctccctgcgctccgaggacac | |
| | | | cgccgtgtacttctgc | |
| huCAN54G2 | H, FR4 | Artificial | tggggccagggcaccctggtgaccgtgtcctccg | 168 |
| | | sequence | | |
| rehuCAN54G2 | K, | Artificial | gacgtggtgatgacccagacccccctgaccctgcccgtgaccctgggcc | 169 |
| | Variable | sequence | agcccgcctccatctcctgcacctcctcccagtccctgctgaactccgac | |
| | region | | ggcgagacctacctgaactggctgctgcagcgccccggccagtccccc | |
| | | | aagcgcctgatccacctggtgtccaagctggactccggcgtgcccgacc | |
| | | | gcatctccggctccggctccggcaccgacttcaccctgaagatctcccgc | |
| | | | gtggaggccgaggacctgggcatctactactgctggcagggcacccact | |
| | | | tcccctggaccttcggcggcggcaccaaggtggagatcaag | |
| rehuCAN54G2 | K, | Artificial | DVVMTQTPLTLPVTLGQPASISCTSSQSLLNSD | 170 |
| | variable | sequence | GETYLNWLLQRPGQSPKRLIHLVSKLDSGVPD | |
| | region | | RISGSGSGTDFTLKISRVEAEDLGIYYCWQGTH | |
| | | | FPWTFGGGTKVEIK | |
| rehuCAN54G2 | K, CDR1 | Artificial | cagtccctgctgaactccgacggcgagacctac | 171 |
| | | sequence | | |
| rehuCAN54G2 | K, CDR2 | Artificial | ctggtgtcc | 172 |
| | | sequence | | |
| rehuCAN54G2 | K, CDR3 | Artificial | tggcagggcacccacttcccctggacc | 173 |
| | | sequence | | |
| rehuCAN54G2 | K, CDR1 | Artificial | QSLLNSDGETY | 174 |
| | | sequence | | |
| rehuCAN54G2 | K, CDR2 | Artificial | LVS | 175 |
| | | sequence | | |
| rehuCAN54G2 | K, CDR3 | Artificial | WQGTHFPWT | 176 |
| | | sequence | | |
| rehuCAN54G2 | K, FR1 | Artificial | gacgtggtgatgacccagacccccctgaccctgcccgtgaccctgggcc | 177 |
| | | sequence | agcccgcctccatctcctgcacctcctcc | |
| rehuCAN54G2 | K, FR2 | Artificial | ctgaactggctgctgcagcgccccggccagtcccccaagcgcctgatcc | 178 |
| | | sequence | ac | |
| rehuCAN54G2 | K, FR3 | Artificial | aagctggactccggcgtgcccgaccgcatctccggctccggctccggc | 179 |
| | | sequence | accgacttcaccctgaagatctcccgcgtggaggccgaggacctgggc | |
| | | | atctactactgc | |
| rehuCAN54G2 | K, FR2 | Artificial | ttcggcggcggcaccaaggtggagatcaag | 180 |
| | | sequence | | |
| rehuCAN54G2 | H, | Artificial | caggtgcagctgcagcagtccggcaccgaggtggtgaagcccggcgc | 181 |
| | Variable | sequence | ctccgtgaagatctcctgcaaggcctccggctacgacttcaccaacttctg | |
| | region | | gctgggctggatcaagcaggcccccggccagggcctggagtggatcg | |
| | | | gcgacatctaccccggcggcgacaacacctactacaacgagaagttcaa | |
| | | | gggcaaggtgaccctgaccgccgacaagtcctccaacaccgcctacat | |
| | | | ggagttctcctccctgcgctccgaggacaccgccgtgtacttctgcttcat | |
| | | | gatcctgtacaccctggactactggggccagggcaccctggtgaccgtg | |
| | | | tcctcc | |
| rehuCAN54G2 | H, | Artificial | QVQLQQSGTEVVKPGASVKISCKASGYDFTNF | 182 |
| | variable | sequence | WLGWIKQAPGQGLEWIGDIYPGGDNTYYNEK | |
| | region | | FKGKVTLTADKSSNTAYMEFSSLRSEDTAVYF | |
| | | | CFMILYTLDYWGQGTLVTVSS | |
| rehuCAN54G2 | H, CDR1 | Artificial | ggctacgacttcaccaacttctgg | 183 |
| | | sequence | | |
| rehuCAN54G2 | H, CDR2 | Artificial | atctaccccggcggcgacaacacc | 184 |
| | | sequence | | |
| rehuCAN54G2 | H, CDR3 | Artificial | ttcatgatcctgtacaccctggactac | 185 |
| | | sequence | | |
| rehuCAN54G2 | H, CDR1 | Artificial | GYDFTNFW | 186 |
| | | sequence | | |
| rehuCAN54G2 | H, CDR2 | Artificial | IYPGGDNT | 187 |
| | | sequence | | |
| rehuCAN54G2 | H, CDR3 | Artificial | FMILYTLDY | 188 |
| | | sequence | | |
| rehuCAN54G2 | H, FR1 | Artificial | caggtgcagctgcagcagtccggcaccgaggtggtgaagcccggcgc | 189 |
| | | sequence | ctccgtgaagatctcctgcaaggcctcc | |
| rehuCAN54G2 | H, FR2 | Artificial | ctgggctggatcaagcaggcccccggccagggcctggagtggatcgg | 190 |
| | | sequence | cgac | |
| rehuCAN54G2 | H, FR3 | Artificial | tactacaacgagaagttcaagggcaaggtgaccctgaccgccgacaagt | 191 |
| | | sequence | cctccaacaccgcctacatggagttctcctccctgcgctccgaggacacc | |
| | | | gccgtgtacttctgc | |
| rehuCAN54G2 | H, FR4 | Artificial | tggggccagggcaccctggtgaccgtgtcctcc | 192 |
| | | sequence | | |
| cdrCAN54G2 | K, | Artificial | gacgtggtgatgacccagtcccccctgtccctgcccgtgaccctgggcc | 193 |
| | Variable | sequence | agcccgcctccatctcctgccgctcctcccagtccctgctgaactccgac | |
| | region | | ggcgagacctacctgaactggttccagcagcgccccggccagtccccc | |
| | | | cgccgcctgatctacctggtgtccaaccgcgactccggcgtgcccgacc | |
| | | | gcttctccggctccggctccggcaccgacttcaccctgaagatctcccgc | |
| | | | gtggaggccgaggacgtgggcgtgtactactgctggcagggcacccac | |
| | | | ttcccctggaccttcggcggcggcaccaaggtggagatcaag | |
| cdrCAN54G2 | K, | Artificial | DVVMTQSPLSLPVTLGQPASISCRSSQSLLNSD | 194 |
| | variable | sequence | GETYLNWFQQRPGQSPRRLIYLVSNRDSGVPD | |
| | region | | RFSGSGSGTDFTLKISRVEAEDVGVYYCWQGT | |
| | | | HFPWTFGGGTKVEIK | |
| cdrCAN54G2 | K, CDR1 | Artificial | cagtccctgctgaactccgacggcgagacctac | 195 |
| | | sequence | | |
| cdrCAN54G2 | K, CDR2 | Artificial | ctggtgtcc | 196 |
| | | sequence | | |
| cdrCAN54G2 | K, CDR3 | Artificial | tggcagggcacccacttcccctggacc | 197 |
| | | sequence | | |
| cdrCAN54G2 | K, CDR1 | Artificial | QSLLNSDGETY | 198 |
| | | sequence | | |
| cdrCAN54G2 | K, CDR2 | Artificial | LVS | 199 |
| | | sequence | | |
| cdrCAN54G2 | K, CDR3 | Artificial | WQGTHFPWT | 200 |
| | | sequence | | |
| cdrCAN54G2 | K, FR1 | Artificial | gacgtggtgatgacccagtcccccctgtccctgcccgtgaccctgggcc | 201 |
| | | sequence | agcccgcctccatctcctgccgctcctcc | |
| cdrCAN54G2 | K, FR2 | Artificial | ctgaactggttccagcagcgccccggccagtccccccgccgcctgatct | 202 |
| | | sequence | ac | |
| cdrCAN54G2 | K, FR3 | Artificial | aaccgcgactccggcgtgcccgaccgcttctccggctccggctccggca | 203 |
| | | sequence | ccgacttcaccctgaagatctcccgcgtggaggccgaggacgtgggcgt | |
| | | | gtactactgc | |
| cdrCAN54G2 | K, FR4 | Artificial | ttcggcggcggcaccaaggtggagatcaag | 204 |
| | | sequence | | |
| cdrCAN54G2 | H, | Artificial | caggtgcagctggtgcagtccggcgccgaggtgaagaagcccggcgc | 205 |
| | Variable | sequence | ctccgtgaaggtgtcctgcaaggcctccggctacgacttcaccaacttctg | |
| | region | | gatgcactgggtgcgccaggcccccggccagggcctggagtggatgg | |
| | | | gcatcatctaccccggcggcgacaacacctcctacgcccagaagttcca | |
| | | | gggccgcgtgaccatgacccgcgacacctccacctccaccgtgtacatg | |
| | | | gagctgtcctccctgcgctccgaggacaccgccgtgtactactgcttcatg | |
| | | | atcctgtacaccctggactactggggccagggcaccctggtgaccgtgtc | |
| | | | ctcc | |
| cdrCAN54G2 | H, | Artificial | QVQLVQSGAEVKKPGASVKVSCKASGYDFTN | 206 |
| | variable | sequence | FWMHWVRQAPGQGLEWMGHYPGGDNTSYA | |
| | region | | QKFQGRVTMTRDTSTSTVYMELSSLRSEDTAV | |
| | | | YYCFMILYTLDYWGQGTLVTVSS | |
| cdrCAN54G2 | H, CDR1 | Artificial | ggctacgacttcaccaacttctgg | 207 |
| | | sequence | | |
| cdrCAN54G2 | H, CDR2 | Artificial | atctaccccggcggcgacaacacc | 208 |
| | | sequence | | |
| cdrCAN54G2 | H, CDR3 | Artificial | ttcatgatcctgtacaccctggactac | 209 |
| | | sequence | | |
| cdrCAN54G2 | H, CDR1 | Artificial | GYDFTNFW | 210 |
| | | sequence | | |
| cdrCAN54G2 | H, CDR2 | Artificial | IYPGGDNT | 211 |
| | | sequence | | |
| cdrCAN54G2 | H, CDR3 | Artificial | FMILYTLDY | 212 |
| | | sequence | | |
| cdrCAN54G2 | H, FR1 | Artificial | caggtgcagctggtgcagtccggcgccgggtgaagaagcccggcgc | 213 |
| | | sequence | ctccgtgaaggtgtcctgcaaggcctcc | |
| cdrCAN54G2 | H, FR2 | Artificial | atgcactgggtgcgccaggcccccggccagggcctggagtggatggg | 214 |
| | | sequence | catc | |
| cdrCAN54G2 | H, FR3 | Artificial | tcctacgcccagaagttccagggccgcgtgaccatgacccgcgacacct | 215 |
| | | sequence | ccacctccaccgtgtacatggagctgtcctccctgcgctccgaggacacc | |
| | | | gccgtgtactactgc | |
| cdrCAN54G2 | H, FR4 | Artificial | tggggccagggcaccctggtgaccgtgtcctcc | 216 |
| | | sequence | | |
| Ravn GPeΔmuc | GPeΔmuc | Synthetic | MKTIYFLISL ILIQSIKTLP VLEIASNSQP | 217 |
| | ΔTM | Marburg | QDVDSVCSGT LQKTEDVHLM GFTLSGQKVA | |
| | | virus | DSPLEASKRW AFRTGVPPKN | |
| | | | VEYTEGEEAK TCYNISVTDP SGKSLLLDPP | |
| | | | SNIRDYPKCK | |
| | | | TVHHIQGQNP HAQGIALHLW | |
| | | | GAFFLYDRVA STTMYRGKVF TEGNIAAMIV | |
| | | | NKTVHRMIFS | |
| | | | RQGQGYRHMN LTSTNKYWTS | |
| | | | SNETQRNDTG CFGILQEYNS TNNQTCPPSL | |
| | | | KPPSLPTVTP | |
| | | | SIHSTNTQIN TAKSGTMRPP IYFRKKRSIF | |
| | | | WKEGDIFPFL DGLINTEIDF DPIPNTETIF | |
| | | | DESPSFNTST NEEQHTPPNI SLTFSYFPDK | |
| | | | NGDTAYSGEN ENDCDAELRI WSVQEDDLAA | |
| | | | GLSWIPFFGP GIEGLYTAGL IKNQNNLVCR | |
| | | | LRRLANQTAK SLELLLRVTT EERTFSLINR | |
| | | | HAIDFLLTRW GGTCKVLGPD CCIGIEDLSK | |
| | | | NISEQIDKIR KDEQKEET | |
| Angola | GPeΔmuc | Synthetic | MKTTCLLISL ILIQGVKTLP ILEIASNIQP | 218 |
| GPeΔmuc | ΔTM | Marburg | QNVDSVCSGT LQKTEDVHLM GFTLSGQKVA | |
| | | virus | DSPLEASKRW AFRAGVPPKN | |
| | | | VEYTEGEEAK TCYNISVTDP SGKSLLLDPP | |
| | | | TNIRDYPKCK | |
| | | | TIHHIQGQNP HAQGIALHL GAFFLYDRIA | |
| | | | STTMYRGKVF TEGNIAAMIV NKTVHKMIFS | |
| | | | RQGQGYRHMN LTSTNKYWTS | |
| | | | SNGTQTNDTG CFGTLQEYNS TKNQTCAPSK | |
| | | | KPLPLPTAHP | |
| | | | EVKLTSTSTD ATKLNTTQHL VYFRRKRNIL | |
| | | | WREGDMFPFL DGLINAPIDF DPVPNTKTIF | |
| | | | DESSSSGASA EEDQHASPNI SLTLSYFPKV | |
| | | | NENTAHSGEN ENDCDAELRI WSVQEDDLAA | |
| | | | GLSWIPFFGP GIEGLYTAGL IKNQNNLVCR | |
| | | | LRRLANQTAK SLELLLRVTT EERTFSLINR | |
| | | | HAIDFLLARW GGTCKVLGPD CCIGIEDLSR | |
| | | | NISEQIDQIK KDEQKEGT | |
| Musoke | GPeΔmuc | Synthetic | MKTTCFLISL ILIQGTKNLP ILEIASNNQP | 219 |
| GPeΔmuc | ΔTM | Marburg | QNVDSVCSGT LQKTEDVHLM GFTLSGQKVA | |
| | | virus | DSPLEASKRW AFRTGVPPKN | |
| | | | VEYTEGEEAK TCYNISVTDP SGKSLLLDPP | |
| | | | TNIRDYPKCK | |
| | | | TIHHIQGQNP HAQGIALHLW GAFFLYDRIA | |
| | | | STTMYRGKVF TEGNIAAMIV NKTVHKMIFS | |
| | | | RQGQGYRHMN LTSTNKYWTS | |
| | | | SNGTQTNDTG CFGALQEYNS TKNQTCAPSK | |
| | | | IPPPLPTART | |
| | | | EIKLTSTPTD ATKLNTTQHL VYFRRKRSIL | |
| | | | WREGDMFPFL DGLINAPIDF DPVPNTKTIF | |
| | | | DESSSSGASA EEDQHASPNI SLTLSYFPNI | |
| | | | NENTAYSGEN ENDCDAELRI WSVQEDDLAA | |
| | | | GLSWIPFFGP GIEGLYTAVL IKNQNNLVCR | |
| | | | LRRLANQTAK SLELLLRVTT EERTFSLINR | |
| | | | HAIDFLLTRW GGTCKVLGPD CCIGIEDLSK | |
| | | | NISEQIDQIK KDEQKEGT | |
| Ci67 GPeΔmuc | GPeΔmuc | Synthetic | MKTTCLFISL ILIQGIKTLP ILEIASNNQP | 220 |
| | ΔTM | Marburg | QNVDSVCSGT LQKTEDVHLM GFTLSGQKVA | |
| | | virus | DSPLEASKRW AFRTGVPPKN | |
| | | | VEYTEGEEAK TCYNISVTDP SGKSLLLDPP | |
| | | | TNIRDYPKCK | |
| | | | TIHHIQGQNP HAQGIALHLW GAFFLYDRIA | |
| | | | STTMYRGRVF TEGNIAAMIV NKTVHKMIFS | |
| | | | RQGQGYRHMN LTSTNKYWTS | |
| | | | NNGTQTNDTG CFGALQEYNS TKNQTCAPSK | |
| | | | IPSPLPTART | |
| | | | EIKPTSTPTD ATTLNTTQHL VYFRKKRSIL | |
| | | | WREGDMFPFL DGLINAPIDF DPVPNTKTIF | |
| | | | DESSSSGASA EEDQHASPNI SLTLSYFPNI | |
| | | | NENTAYSGEN ENDCDAELRI WSVQEDDLAA | |
| | | | GLSWIPFFGP GIEGLYTAGL IKNQNNLVCR | |
| | | | LRRLANQTAK SLELLLRVTT EERTFSLINR | |
| | | | HAIDFLLTRW GGTCKVLGPD CCIGIEDLSR | |
| | | | NISEQIDQIK KDEQKEGT | |
| CNA30G5 | K, | Murine | gatattgtgctgacccaatctccactctccctgcctgtcagtcttggagatc | 221 |
| | Variable | sequence | aagcctccatctcttgcagatctagtcagagccttgtacacagtaatggaa | |
| | region | | acacctatttacattggtacctgcagaagccaggccagtctccaaacctcc | |
| | | | tgatctacaaagtttccaaccgattttctggggtcccagacaggttcagtgg | |
| | | | cagtggatcagggacagatttcacactcaagatcagcagagtggaggct | |
| | | | gaggatctgggagtttatttctgctctcaaagtacacatgttccgtggacgtt | |
| | | | cggtggaggcaccaagctggaaatcaaa | |
| CAN30G5 | K, | Murine | DIVLTQSPLSLPVSLGDQASISCRSSQSLVHSNG | 222 |
| | variable | sequence | NTYLHWYLQKPGQSPNLLIYKVSNRFSGVPDR | |
| | region | | FSGSGSGTDFTLKISRVEAEDLGVYFCSQSTHV | |
| | | | PWTFGGGTKLEIK | |
| CAN30G5 | K, CDR1 | Murine | cagagccttgtacacagtaatggaaacacctat | 223 |
| | | sequence | | |
| CAN30G5 | K, CDR2 | Murine | aaagtttcc | 224 |
| | | sequence | | |
| CAN30G5 | K, CDR3 | Murine | tctcaaagtacacatgttccgtggacg | 225 |
| | | sequence | | |
| CAN30G5 | K, CDR1 | Murine | QSLVHSNGNTY | 226 |
| | | sequence | | |
| CAN30G5 | K, CDR2 | Murine | KVS | 227 |
| | | sequence | | |
| CAN30G5 | K, CDR3 | Murine | SQSTHVPWT | 228 |
| | | sequence | | |
| CAN30G5 | H, | Murine | gaggtgcagcttgttgagtctggtggaggattggtgcagcctaaaggatc | 229 |
| | Variable | sequence | attgaaactctcatgtgccgcctctggtttcgccttcaattcctatgccatgc | |
| | region | | actgggtctgccaggctccaggaaagggtttggaatgggttgctcgcata | |
| | | | agaattaaaagtggtaattatgcaacatcttatgccggttcagtgacagaca | |
| | | | gattcaccgtctccagagatgattcacaaaacttgttctatctgcaaatgaa | |
| | | | caacctgaaaactgaggacacagccatgtattactgtgtgagagagtggg | |
| | | | aaggggctatggactactggggtcaaggaacctcagtcaccgtctcctca | |
| | | | g | |
| CAN30G5 | H, | Murine | EVQLVESGGGLVQPKGSLKLSCAASGFAFNSY | 230 |
| | variable | sequence | AMHWVCQAPGKGLEWVARIRIKSGNYATSYA | |
| | region | | GSVTDRFTVSRDDSQNLFYLQMNNLKTEDTA | |
| | | | MYYCVREWEGAMDYWGQGTSVTVSS | |
| CAN30G5 | H, CDR1 | Murine | ggtttcgccttcaattcctatgcc | 231 |
| | | sequence | | |
| CAN30G5 | H, CDR2 | Murine | ataagaattaaaagtggtaattatgcaaca | 232 |
| | | sequence | | |
| CAN30G5 | H, CDR3 | Murine | gtgagagagtgggaaggggctatggactac | 233 |
| | | sequence | | |
| CAN30G5 | H, CDR1 | Murine | GFAFNSYA | 234 |
| | | sequence | | |
| CAN30G5 | H, CDR2 | Murine | IRIKSGNYAT | 235 |
| | | sequence | | |
| CAN30G5 | H, CDR3 | Murine | VREWEGAMDY | 236 |
| | | sequence | | |
| CAN54G2 | K, | Murine | gatgttgtgatgacccagactcccctcactttgtcggttaccattggacagc | 237 |
| | Variable | sequence | cagcctccatctcttgcacgtcaagtcagagcctcttaaatagtgatgggg | |
| | region | | agacatatttgaattggttgttacagaggccaggccagtctccaaagcgcc | |
| | | | taatccatctggtgtctaaactggactctggagtccctgacaggatcactg | |
| | | | gcagtggatctgggacagatttcacactgaaaatcaacagagtggaggct | |
| | | | gaggatttgggaatttattattgctggcaaggtacacattttccgtggacgtt | |
| | | | cggtggaggcaccaagctggaaatcaaac | |
| CAN54G2 | K, | Murine | DVVMTQTPLTLSVTIGQPASISCTSSQSLLNSDG | 238 |
| | variable | sequence | ETYLNWLLQRPGQSPKRLIHLVSKLDSGVPDRI | |
| | region | | TGSGSGTDFTLKINRVEAEDLGIYYCWQGTHF | |
| | | | PWTFGGGTKLEIK | |
| CAN54G2 | | Murine | cagagcctcttaaatagtgatggggagacatat | 239 |
| | | sequence | | |
| CAN54G2 | | Murine | ctggtgtct | 240 |
| | | sequence | | |
| CAN54G2 | | Murine | tggcaaggtacacattttccgtggacg | 241 |
| | | sequence | | |
| CAN54G2 | | Murine | WSLLNSDGETY | 242 |
| | | sequence | | |
| CAN54G2 | | Murine | LVS | 243 |
| | | sequence | | |
| CAN54G2 | | Murine | WQGTHFPWT | 244 |
| | | sequence | | |
| CAN54G2 | H, | Murine | caggtccagttgcagcagtctggaactgagctggtaaggcctgggacttc | 245 |
| | Variable | sequence | agtgaagatatcctgcaaggcttctggatacgacttcactaacttttggcta | |
| | region | | ggttggataaagcagaggcctggacatggacttgaatggattggagatat | |
| | | | ttaccctggaggtgataatacttactacaatgagaagttcaagggcaaagt | |
| | | | cacgctgactgcagacaaatcctcgaacacagcctatatgcagttcagtc | |
| | | | gcctgacatctgaggactctgctgtctatttctgttttatgattctctatactttg | |
| | | | gactactggggtcaaggaacctcagtcacgtctcctcag | |
| CAN54G2 | H, | Murine | QVQLQQSGTELVRPGTSVKISCKASGYDFTNF | 246 |
| | variable | sequence | WLGWIKQRPGHGLEWIGDIYPGGDNTYYNEK | |
| | region | | FKGKVTLTADKSSNTAYMQFSSLTSEDSAVYF | |
| | | | CFMILYTLDYWGQGTSVTVSS | |
| CAN54G2 | H, CDR1 | Murine | ggatacgacttcactaacttttgg | 247 |
| | | sequence | | |
| CAN54G2 | H, CDR2 | Murine | atttaccctggaggtgataatact | 248 |
| | | sequence | | |
| CAN54G2 | H, CDR3 | Murine | tttatgattctctatactttggactac | 249 |
| | | sequence | | |
| CAN54G2 | H, CDR1 | Murine | GYDFTNFW | 250 |
| | | sequence | | |
| CAN54G2 | H, CDR2 | Murine | IYPGGDNT | 251 |
| | | sequence | | |
| CAN54G2 | H, CDR3 | Murine | FMILYTLDY | 252 |
| | | sequence | | |
| CAN30G2 | K, | Murine | gatgttttgatgacccaaactccactctccctgcctgtcagtcttggagatc | 253 |
| | Variable | sequence | aagcctccatctcttgcagatctagtcagagcattgtacatagtaatggaga | |
| | region | | cacctatttagaatggtacctgcagaaaccaggccagtctccaaagctcct | |
| | | | gatctacaaagtttccaaccgattttctggggtcccagacaggttcagtgg | |
| | | | cagtggatcagggacagatttcacactcaggatcagcagagtggaggct | |
| | | | gaggatctgggagtttattactgctttcaaggttcacattttccgtggacgtt | |
| | | | cggtggaggcaccaagctggaaatcaaac | |
| CAN30G2 | K, | Murine | DVLMTQTPLSLPVSLGDQASISCRSSQSIVHSN | 254 |
| | variable | sequence | GDTYLEWYLQKPGQSPKLLIYKVSNRFSGVPD | |
| | region | | RFSGSGSGTDFTLRISRVEAEDLGVYYCFQGSH | |
| | | | FPWTFGGGTKLEIK | |
| CAN30G2 | K, CDR1 | Murine | cagagcattgtacatagtaatggagacacctat | 255 |
| | | sequence | | |
| CAN30G2 | K, CDR2 | Murine | aaagtttcc | 256 |
| | | sequence | | |
| CAN30G2 | K, CDR3 | Murine | tttcaaggttcacattttccgtggacg | 257 |
| | | sequence | | |
| CAN30G2 | K, CDR1 | Murine | QSIVHSNGDTY | 258 |
| | | sequence | | |
| CAN30G2 | K, CDR2 | Murine | KVS | 259 |
| | | sequence | | |
| CAN30G2 | K, CDR3 | Murine | FQGSHFPWT | 260 |
| | | sequence | | |
| CAN30G2 | H, | Murine | gatgtgcagctggtggagtctgggggaggcttagtgcagcctggagggt | 261 |
| | Variable | sequence | cccggaaactctcctgtacagcctctggattcactttcagtagctttggaat | |
| | region | | gcactgggttcgtcaggctccagagaaggggctggagtgggtcgcatac | |
| | | | attagtagtggcagtagtaaaatctactatgcagacacggtgaagggccg | |
| | | | attcaccatctccagagacaatcccaagazacaccctgttcctgcaaatgac | |
| | | | cagtctaaggtctgaggacacggccatgtattactgtgcaagagggtggt | |
| | | | acgagggggcctggtttgcttactggggccaagggactctggtcactgtc | |
| | | | tctgcag | |
| CAN30G2 | H, | Murine | DVQLVESGGGLVQPGGSRKLSCTASGFTFSSFG | 262 |
| | variable | sequence | MHWVRQAPEKGLEWVAYISSGSSKIYYADTV | |
| | region | | KGRFTISRDNPKNTLFLQMTSLRSEDTAMYYC | |
| | | | ARGWYEGAWFAYWGQGTLVTVSA | |
| CAN30G2 | H, CDR1 | Murine | ggattcactttcagtagctttgga | 263 |
| | | sequence | | |
| CAN30G2 | H, CDR2 | Murine | attagtagtggcagtagtaaaatc | 264 |
| | | sequence | | |
| CAN30G2 | H, CDR3 | Murine | gcaagagggtggtacgagggggcctggtttgcttac | 265 |
| | | sequence | | |
| CAN30G2 | H, CDR1 | Murine | GFTFSSFG | 266 |
| | | sequence | | |
| CAN30G2 | H, CDR2 | Murine | ISSGSSKI | 267 |
| | | sequence | | |
| CAN30G2 | H, CDR3 | Murine | ARGWYEGAWFAY | 268 |
| | | sequence | | |
| CAN40G1 | K, | Murine | gacattgtgctgacccaatctccagcttctttggctgtgtctctagggcaga | 269 |
| | Variable | sequence | gggcctccatctcctgcaaggccagccaaagtgttgatcatgatggtgat | |
| | region | | agttatatgaactggtaccaacagaaaccaggacagccacccaaactcct | |
| | | | catctatgctacatccaatctagaatctgggatcccagccaggtttagtggc | |
| | | | agtgggtctgggacagacttcaccctcaacatccatcctgtggaggagga | |
| | | | ggatgctgcaacctattactgtcagcagagttatgaggttccgctcacgttc | |
| | | | ggtgctgggaccaagctggagctgaaac | |
| CAN40G1 | K, | Murine | DIVLTQSPASLAVSLGQRASISCKASQSVDHDG | 270 |
| | variable | sequence | DSYMNWYQQKPGQPPKLLIYATSNLESGIPAR | |
| | region | | FSGSGSGTDFTLNIHPVEEEDAATYYCQQSYEV | |
| | | | PLTFGAGTKLELK | |
| CAN40G1 | K, CDR1 | Murine | caaagtgttgatcatgatggtgatagttat | 271 |
| | | sequence | | |
| CAN40G1 | K, CDR2 | Murine | gctacatcc | 272 |
| | | sequence | | |
| CAN40G1 | K, CDR3 | Murine | cagcagagttatgaggttccgctcacg | 273 |
| | | sequence | | |
| CAN40G1 | K, CDR1 | Murine | QSVDHDGDSY | 274 |
| | | sequence | | |
| CAN40G1 | K, CDR2 | Murine | ATS | 275 |
| | | sequence | | |
| CAN40G1 | K, CDR3 | Murine | QQSYEVPLT | 276 |
| | | sequence | | |
| CAN40G1 | H, | Murine | gaggtccagctgcaacagtctggacctgagctggtgaagcctggggctt | 277 |
| | Variable | sequence | cagtgaagatatcctgcaggacttctggatacacattcactgaatacacca | |
| | region | | ttcactgggtgaagcagagccgtggaaagagccttgagtggattggagg | |
| | | | tattaatcctaaccatggtggtactctctacaaccagaagttcaaggtcaag | |
| | | | gccacattgactgtagacaagtcctccagcacagcctacatggagctccg | |
| | | | cagcctgacatctgaggattctgcagtctattactgtgcaagatttacttacg | |
| | | | actactggggccaaggcaccactctcacagtctcctcag | |
| CAN40G1 | H, | Murine | EVQLQQSGPELVKPGASVKISCRTSGYTFTEYT | 278 |
| | variable | sequence | IHWVKQSRGKSLEWIGGINPNHGGTLYNQKFK | |
| | region | | VKATLTVDKSSSTAYMELRSLTSEDSAVYYCA | |
| | | | RFTYDYWGQGTTLTVSS | |
| CAN40G1 | H, CDR1 | Murine | ggatacacattcactgaatacacc | 279 |
| | | sequence | | |
| CAN40G1 | H, CDR2 | Murine | attaatcctaaccatggtggtact | 280 |
| | | sequence | | |
| CAN40G1 | H, CDR3 | Murine | gcaagatttacttacgactac | 281 |
| | | sequence | | |
| CAN40G1 | H, CDR1 | Murine | GYTFTEYT | 282 |
| | | sequence | | |
| CAN40G1 | H, CDR2 | Murine | INPNHGGT | 283 |
| | | sequence | | |
| CAN40G1 | H, CDR3 | Murine | ARFTYDY | 284 |
| | | sequence | | |
| CAN30G1 | K, | Murine | gaaaatgttctcacccagtctccagcaatcatgtctgcatctccaggggaa | 285 |
| | Variable | sequence | aaggtcaccatgacctgcagtgccagctcaagtgtaacttacatgcactg | |
| | region | | gtaccagcagaagtcaagcacctcccccaaactctggatttatgacacat | |
| | | | ccaaactggcttctggagtcccaggtcgcttcagtggcagtgggtctgga | |
| | | | aactcttactctctcacgatcagcagcatggaggctgaagatgttgccactt | |
| | | | attactgttttcaggggagtgggtacccgtacacgttcggaggggggacc | |
| | | | aagctggaaataaaac | |
| CAN30G1 | K, | Murine | ENVLTQSPAIMSASPGEKVTMTCSASSSVTYM | 286 |
| | variable | sequence | HWYQQKSSTSPKLWIYDTSKLASGVPGRFSGS | |
| | region | | GSGNSYSLTISSMEAEDVATYYCFQGSGYPYTF | |
| | | | GGGTKLEIK | |
| CAN30G1 | K, CDR1 | Murine | gccagctcaagtgtaacttac | 287 |
| | | sequence | | |
| CAN30G1 | K, CDR2 | Murine | gacacatcc | 288 |
| | | sequence | | |
| CAN30G1 | K, CDR3 | Murine | tttcaggggagtgggtacccgtacacg | 289 |
| | | sequence | | |
| CAN30G1 | K, CDR1 | Murine | ASSSVTY | 290 |
| | | sequence | | |
| CAN30G1 | K, CDR2 | Murine | CTS | 291 |
| | | sequence | | |
| CAN30G1 | K, CDR3 | Murine | FQGSGYPYT | 292 |
| | | sequence | | |
| CAN30G1 | H, | Murine | cagatccagttggtgcagtctggacctgagctgaagaagcctggagaga | 293 |
| | Variable | sequence | cagtcaagatctcctgcaaggcttctgggtataccttcacaaactatggaat | |
| | region | | gaactgggtgaagcaggctccaggaaagggtttaaagtggatgggctg | |
| | | | gataaacacctacactggaaagccaacatatgttgatgacttcaagggac | |
| | | | ggtttgccttctctttggaaacctctgccaacactgcctatttgcagatcaac | |
| | | | aacctcaaaaatgaggacacggctacatatttctgtgaaagtggaggttac | |
| | | | gacgaggactactggggccaaggcaccactctcacagtctcctcag | |
| CAN30G1 | H, | Murine | QIQLVQSGPELKKPGETVKISCKASGYTFTNYG | 294 |
| | variable | sequence | MNWVKQAPGKGLKWMGWINTYGKPTYVD | |
| | region | | DFKGRFAFSLETSANTAYLQINNLKNEDTATYF | |
| | | | CESGGYDEDYWGQGTTLTVSS | |
| CAN30G1 | H, CDR1 | Murine | gggtataccttcacaaactatgga | 295 |
| | | sequence | | |
| CAN30G1 | H, CDR2 | Murine | ataaacacctacactggaaagcca | 296 |
| | | sequence | | |
| CAN30G1 | H, CDR3 | Murine | gaaagtggaggttacgacgaggactac | 297 |
| | | sequence | | |
| CAN30G1 | H, CDR1 | Murine | GYTFTNYG | 298 |
| | | sequence | | |
| CAN30G1 | H, CDR2 | Murine | INTYTGKP | 299 |
| | | sequence | | |
| CAN30G1 | H, CDR3 | Murine | ESGGYDEDY | 300 |
| | | sequence | | |
| CAN30G3 | K, | Murine | gatgtttgatgacccaaactccactctccctgcctgtcagtcttggagatc | 301 |
| | Variable | sequence | aagcctccatctcttgcagatctagtcagaacattgtacatagtaatggaaa | |
| | region | | cacctatttagaatggtacctgcagaaatcaggccagtctccaaagctcct | |
| | | | gatctacaaagtttccaaccgattttctggggtcccagacaggttcagtgg | |
| | | | cagtggatcagggacagatttcacactcaagatcagcagagtggaggct | |
| | | | gaggatctgggagtttattactgctttcaaggttcacattttccgtggacgtt | |
| | | | cggtggaggcaccaagctggaaatcaaac | |
| CAN30G3 | K, | Murine | DVLMTQTPLSLPVSLGDQASISCRSSQNIVHSN | 302 |
| | variable | sequence | GNTYLEWYLQKSGQSPKLLIYKVSNRFSGVPD | |
| | region | | RFSGSGSGTDFTLKISRVEAEDLGVYYCFQGSH | |
| | | | FPWTFGGGTKLEIK | |
| CAN30G3 | K, CDR1 | Murine | cagaacattgtacatagtaatggaaacacctat | 303 |
| | | sequence | | |
| CAN30G3 | K, CDR2 | Murine | aaagtttcc | 304 |
| | | sequence | | |
| CAN30G3 | K, CDR3 | Murine | tttcaaggttcacattttccgtggacg | 305 |
| | | sequence | | |
| CAN30G3 | K, CDR1 | Murine | QNIVHSNGNTY | 306 |
| | | sequence | | |
| CAN30G3 | K, CDR2 | Murine | KVS | 307 |
| | | sequence | | |
| CAN30G3 | K, CDR3 | Murine | FQGSHFPWT | 308 |
| | | sequence | | |
| CAN30G3 | H, | Murine | caggtgcagctgaaggagtcaggacctggcctggtggcgccctcacag | 309 |
| | Variable | sequence | agcctgtccatcacatgcactgtctcagggttctccttaaccgactatggta | |
| | region | | taacctggattcgccagcctccaggaaagggtctggagtggctgggagt | |
| | | | aatatggggtggtggaagcgcatactataatttagttctcaaatccagactg | |
| | | | agcatcagcaaggacaactccaagagtcaagttttcttaaaaatgaacagt | |
| | | | ctgcaaactgatgacacagccatgtactactgtgccaaacatcggactttg | |
| | | | attacgactgctatggactactggggtcaaggaatttcagtcaccgtctcct | |
| | | | cag | |
| CAN30G3 | H, | Murine | QVQLKESGPGLVAPSQSLSITCTVSGFSLTDYGI | 310 |
| | variable | sequence | TWIRQPPGKGLEWLGVIWGGGSAYYNLVLKS | |
| | region | | RLSISKDNSKSQVFLKMNSLQTDDTAMYYCAK | |
| | | | HRTLITTAMDYWGQGISVTVSS | |
| CAN30G3 | H, CDR1 | Murine | gggttctccttaaccgactatggt | 311 |
| | | sequence | | |
| CAN30G3 | H, CDR2 | Murine | atatggggtggtggaagcgca | 312 |
| | | sequence | | |
| CAN30G3 | H, CDR3 | Murine | gccaaacatcggactttgattacgactgctatggactac | 313 |
| | | sequence | | |
| CAN30G3 | H, CDR1 | Murine | GFSLTDYG | 314 |
| | | sequence | | |
| CAN30G3 | H, CDR2 | Murine | IWGGGSA | 315 |
| | | sequence | | |
| CAN30G3 | H, CDR3 | Murine | AKHRTLITTAMDY | 316 |
| | | sequence | | |
| CAN30G4 | K, | Murine | gatgtttgatgacccaaactccactctccctgcctgtcagtcttggagatc | 317 |
| | Variable | sequence | aagcctccatctcttgcagatctagtcagaacattgtacatagtgatggaaa | |
| | region | | cacctatttagaatggtacctgcagaaaccaggccagtctccaaagctcct | |
| | | | gatctacaaattttccaaccgattttctggggtcccagacaggttcagtggc | |
| | | | agtggatcagggacagatttcacactcaagatcagcagagtggaggctg | |
| | | | aggatctgggagtttattactgctttcaaggttcacatgttcctcccacgttc | |
| | | | ggtgctgggaccaagctggagctgaaac | |
| CAN30G4 | K, | Murine | DVLMTQTPLSLPVSLGDQASISCRSSQNIVHSD | 318 |
| | variable | sequence | GNTYLEWYLQKPGQSPKLLIYKFSNRFSGVPD | |
| | region | | RFSGSGSGTDFTLKISRVEAEDLGVYYCFQGSH | |
| | | | VPPTFGAGTKLELK | |
| CAN30G4 | K, CDR1 | Murine | cagaacattgtacatagtgatggaaacacctat | 319 |
| | | sequence | | |
| CAN30G4 | K, CDR2 | Murine | aaattttcc | 320 |
| | | sequence | | |
| CAN30G4 | K, CDR3 | Murine | tttcaaggttcacatgttcctcccacg | 321 |
| | | sequence | | |
| CAN30G4 | K, CDR1 | Murine | QNIVHSDGNTY | 322 |
| | | sequence | | |
| CAN30G4 | K, CDR2 | Murine | KFS | 323 |
| | | sequence | | |
| CAN30G4 | K, CDR3 | Murine | FQGSHVPPT | 324 |
| | | sequence | | |
| CAN30G4 | H, | Murine | gaagtgaagctggtggagtctggggaggcttagtgaagtctggagggt | 325 |
| | Variable | sequence | ccctgaaactctcctgtgcagtctctggattcactttcagtacctatgccatg | |
| | region | | tcttgggttcgccagattccggagaagaggctggagtgggtcgcaaccat | |
| | | | tagtaatggtggtagttatatctactattcagacagtgtgaagggtcgattca | |
| | | | ccatctccagagacaatgccaagaacaccctgtacctgcaaatgagcagt | |
| | | | ctgaggtctgaggacacggccatgtattactgtgcacgacatagggagtc | |
| | | | ctataggtacgactggtttgcttactggggccaagggactctggtcactgt | |
| | | | ctctgcag | |
| CAN30G4 | H, | Murine | EVKLVESGGGLVKSGGSLKLSCAVSGFTFSTY | 326 |
| | variable | sequence | AMSWVRQIPEKRLEWVTAISNGGSYIYYSDSV | |
| | region | | KGRFTISRDNAKNTLYLQMSSLRSEDTAMYYC | |
| | | | ARHRESYRYDWFAYWGQGTLVTVSA | |
| CAN30G4 | H, CDR1 | Murine | ggattcactttcagtacctatgcc | 327 |
| | | sequence | | |
| CAN30G4 | H, CDR2 | Murine | attagtaatggtggtagttatatc | 328 |
| | | sequence | | |
| CAN30G4 | H, CDR3 | Murine | gcacgacatagggagtcctataggtacgactggtttgcttac | 329 |
| | | sequence | | |
| CAN30G4 | H, CDR1 | Murine | GFTFSTYA | 330 |
| | | sequence | | |
| CAN30G4 | H, CDR2 | Murine | ISNGGSYI | 331 |
| | | sequence | | |
| CAN30G4 | H, CDR3 | Murine | ARHRESYRYDWFAY | 332 |
| | | sequence | | |
| huCAN30G5 | K, FR1 | Artificial | DIVLTQSPLSLPVTLGQPASISCRSS | 333 |
| | | sequence | | |
| huCAN30G5 | K, FR2 | Artificial | LHWYQQRPGQSPRLLIY | 334 |
| | | sequence | | |
| huCAN30G5 | K, FR3 | Artificial | NRFSGVPDRFSGSGSGTDFTLKISRVEAEDVGV | 335 |
| | | sequence | YYC | |
| huCAN30G5 | K, FR4 | Artificial | FGGGTKVEIK | 336 |
| | | sequence | | |
| huCAN30G5 | H, FR1 | Artificial | EVQLVESGGGLVQPGGSLKLSCAAS | 337 |
| | | sequence | | |
| huCAN30G5 | H, FR2 | Artificial | MHWVRQASGKGLEWVAR | 338 |
| | | sequence | | |
| huCAN30G5 | H, FR3 | Artificial | SYAGSVKGRFTVSRDDSKNTFYLQMNSLKTED | 339 |
| | | sequence | TAMYYS | |
| huCAN30G5 | H, FR4 | Artificial | WGQGTLVTVSS | 340 |
| | | sequence | | |
| huCAN30G5 | K, FR1 | Artificial | DIVLTQSPLSLPVTLGQPASISCRSS | 341 |
| | | sequence | | |
| huCAN30G5 | K, FR2 | Artificial | LHWYLQKPGQSPRLLIY | 342 |
| | | sequence | | |
| huCAN30G5 | K, FR3 | Artificial | NRFSGVPDRFSGSGSGTDFTLKISRVEAEDVGV | 343 |
| | | sequence | YFC | |
| huCAN30G5 | K, FR4 | Artificial | FGGGTKLEIK | 344 |
| | | sequence | | |
| huCAN30G5 | H, FR1 | Artificial | EVQLVESGGGLVQPGGSLKLSCAAS | 345 |
| | | sequence | | |
| huCAN30G5 | H, FR2 | Artificial | MHWVCQASGKGLEWVAR | 346 |
| | | sequence | | |
| huCAN30G5 | H, FR3 | Artificial | SYAGSVKGRFTVSRDDSKSLFYLQMNNLKTED | 347 |
| | | sequence | TAMYYC | |
| huCAN30G5 | H, FR4 | Artificial | WQGQTLVTVSS | 348 |
| | | sequence | | |
| huCAN30G5 | K, FR1 | Artificial | DVVMTQSPLSLPVTLGQPASISCRSS | 349 |
| | | sequence | | |
| huCAN30G5 | K, FR2 | Artificial | LNWFQQRPGQSPRRLIY | 350 |
| | | sequence | | |
| huCAN30G5 | K, FR3 | Artificial | NRDSGVPDRFSGSGSGTDFTLKISRVEAEDVGV | 351 |
| | | sequence | YYC | |
| huCAN30G5 | K, FR4 | Artificial | FGGGTKVEIK | 352 |
| | | sequence | | |
| huCAN30G5 | H, FR1 | Artificial | EVQLVESGGGLVQPGGSLKLSCAAS | 353 |
| | | sequence | | |
| huCAN30G5 | H, FR2 | Artificial | MHWVRQASGKGLEWVGR | 354 |
| | | sequence | | |
| huCAN30G5 | H, FR3 | Artificial | AYAASVKGRFTISRDDSKNTAYLQMNSLKTED | 355 |
| | | sequence | TAVYYC | |
| huCAN30G5 | H, FR4 | Artificial | WGWGTLVTVSS | 356 |
| | | sequence | | |
| huCAN40G1 | K, FR1 | Artificial | DIVLTQSPASLAVSLGERATISCKAS | 357 |
| | | sequence | | |
| huCAN40G1 | K, FR2 | Artificial | MNWYQQKPGQPPKLLIY | 358 |
| | | sequence | | |
| huCAN40G1 | K, FR3 | Artificial | NLESGIPARFSGSGSGTDFTLTISSLQAEDVATY | 359 |
| | | sequence | YC | |
| huCAN40G1 | K, FR4 | Artificial | FGAGTKLEIK | 360 |
| | | sequence | | |
| huCAN40G1 | H, FR1 | Artificial | EVQLQQSGPEVKKPGASVKVSCRTS | 361 |
| | | sequence | | |
| huCAN40G1 | H, FR2 | Artificial | IHWVKQAPGKGLEWIGG | 362 |
| | | sequence | | |
| huCAN40G1 | H, FR3 | Artificial | LYNQKFKGRVTLTVDKSSSTAYMELSRLRSDD | 363 |
| | | sequence | TAVYYC | |
| huCAN40G1 | H, FR4 | Artificial | WGQGTLVTVSS | 364 |
| | | sequence | | |
| rehuCAN40G1 | K, FR1 | Artificial | DIVLTQSPASLAVSLGERATISCKAS | 365 |
| | | sequence | | |
| rehuCAN40G1 | K, FR2 | Artificial | MNWYQQKPGQPPKLLIY | 366 |
| | | sequence | | |
| rehuCAN40G1 | K, FR3 | Artificial | NLESGIPARFSGSGSGTDFTLNISSVQAEDVAT | 367 |
| | | sequence | YYC | |
| rehuCAN40G1 | K, FR4 | Artificial | FGAGTKLELK | 368 |
| | | sequence | | |
| rehuCAN40G1 | H, FR1 | Artificial | EVQLQQSGPEVVKPGASVKISCRTS | 369 |
| | | sequence | | |
| rehuCAN40G1 | H, FR2 | Artificial | IHWVKQAPGKGLEWIGG | 370 |
| | | sequence | | |
| rehuCAN40G1 | H, FR3 | Artificial | LYNQKFKGRATLTVDKSSSTAYMELSRLRSDD | 371 |
| | | sequence | TAVYYC | |
| rehuCAN40G1 | H, FR4 | Artificial | WGQGTLLTVSS | 372 |
| | | sequence | | |
| cdrCAN40G1 | K, FR1 | Artificial | DIVMTQSPDSLAVSLGERATINCKSS | 373 |
| | | sequence | | |
| cdrCAN40G1 | K, FR2 | Artificial | LAWYQQKPGQPPKLLIY | 374 |
| | | sequence | | |
| cdrCAN40G1 | K, FR3 | Artificial | TRESGVPDRFSGSGSGTDFTLTISSLQAEDVAV | 375 |
| | | sequence | YYC | |
| cdrCAN40G1 | K, FR4 | Artificial | FGQGTKLEIK | 376 |
| | | sequence | | |
| cdrCAN40G1 | H, FR1 | Artificial | QVQLVQSGAEVKKPGASVKVSCKAS | 377 |
| | | sequence | | |
| cdrCAN40G1 | H, FR2 | Artificial | MHWVRQAPGQGLEWMGW | 378 |
| | | sequence | | |
| cdrCAN40G1 | H, FR3 | Artificial | NYAQKFQGRVTMTRDTSISTAYMELSRLRSDD | 379 |
| | | sequence | TAVYYC | |
| cdrCAN40G1 | H, FR4 | Artificial | WGQGTLVTVSS | 380 |
| | | sequence | | |
| huCAN54G2 | K, FR1 | Artificial | DVVMTQTPLTLPVTLGQPASISCTSS | 381 |
| | | sequence | | |
| huCAN54G2 | K, FR2 | Artificial | LNWLLQRPGQSPKRLHI | 382 |
| | | sequence | | |
| huCAN54G2 | K, FR3 | Artificial | KLDSGVPDRFSGSGSGTDFTLKISRVEAEDVGV | 383 |
| | | sequence | YYC | |
| huCAN54G2 | K, FR4 | Artificial | FGGGTKVEIK | 384 |
| | | sequence | | |
| huCAN54G2 | H, FR1 | Artificial | QVQLQQSGTEVKKPGASVKVSCKAS | 385 |
| | | sequence | | |
| huCAN54G2 | H, FR2 | Artificial | LGWIRQAPGQGLEWIGD | 386 |
| | | sequence | | |
| huCAN54G2 | H, FR3 | Artificial | YYNEKFKGRVTLTADKSSNTAYMELSSLRSED | 387 |
| | | sequence | TAVYFC | |
| huCAN54G2 | H, FR4 | Artificial | WGQGTLVTVSS | 388 |
| | | sequence | | |
| rehuCAN54G2 | K, FR1 | Artificial | DVVMTQTPLTLPVTLGQPASISCTSS | 389 |
| | | sequence | | |
| rehuCAN54G2 | K, FR2 | Artificial | LNWLLQRPGQSPKRLHI | 390 |
| | | sequence | | |
| rehuCAN54G2 | K, FR3 | Artificial | KLDSGVPDRISGSGSGTDFTLKISRVEAEDLGIY | 391 |
| | | sequence | YC | |
| rehuCAN54G2 | K, FR4 | Artificial | FGGGTKVEIK | 392 |
| | | sequence | | |
| rehuCAN54G2 | H, FR1 | Artificial | QVQLQQSGTEVVKPGASVKISCKAS | 393 |
| | | sequence | | |
| rehuCAN54G2 | H, FR2 | Artificial | LGWIKQAPGQGLEWIGD | 394 |
| | | sequence | | |
| rehuCAN54G2 | H, FR3 | Artificial | YYNEKFKGKVTLTADKSSNTAYMEFSSLRSED | 395 |
| | | sequence | TAVYFC | |
| rehuCAN54G2 | H, FR4 | Artificial | WGQGTLVTVSS | 396 |
| | | sequence | | |
| cdrCAN54G2 | K, FR1 | Artificial | DVVMTQSPLSLPVTLGQPASISCRSS | 397 |
| | | sequence | | |
| cdrCAN54G2 | K, FR2 | Artificial | LNWFQQRPGQSPRRLIY | 398 |
| | | sequence | | |
| cdrCAN54G2 | K, FR3 | Artificial | NRDSGVPDRFSGSGSGTDFTLKISRVEAEDVGV | 399 |
| | | sequence | YYC | |
| cdrCAN54G2 | K, FR4 | Artificial | FGGGTKVEIK | 400 |
| | | sequence | | |
| cdrCAN54G2 | H, FR1 | Artificial | QVQLVQSGAEVKKPGASVKVSCKAS | 401 |
| | | sequence | | |
| cdrCAN54G2 | H, FR2 | Artificial | MHWVRQAPGQGLEWMGI | 402 |
| | | sequence | | |
| cdrCAN54G2 | H, FR3 | Artificial | SYAQKFQGRVTMTRDTSTSTVYMELSSLRSED | 403 |
| | | sequence | TAVYYC | |
| cdrCAN54G2 | H, FR4 | Artificial | WGQGTLVTVSS | 404 |
| | | sequence | | |

In one embodiment, an anti-filovirus antibody or antigen-binding portion thereof is a humanized antibody of a murine anti-filovirus antibody. The humanized anti-filovirus antibody or antigen-binding fragment thereof can comprise one or more CDRs of a murine anti-filovirus antibody and one or more human FRs. In one embodiment, the humanized anti-filovirus antibody or antigen-binding fragment thereof comprises one or more CDRs of the murine anti-filovirus antibody CAN30G5, CAN54G2, CAN30G2, CAN40G1, CAN30G1, CAN30G3, or CAN30G4. In one embodiment, the humanized anti-filovirus antibody or antigen-binding fragment thereof comprises one or more human FRs from Table 2.

In one embodiment, a humanized anti-filovirus antibody or antigen-binding portion thereof comprises a light chain CDR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 6, 30, 54, 78, 102, 126, 150, 174 or 198; a light chain CDR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 7, 31, 55, 79, 103, 127, 151, 175, or 199; a light chain CDR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 8, 32, 56, 80, 104, 128, 152, 176, or 200; a heavy chain CDR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 18, 42, 66, 90, 114, 138, 162, 186, or 210; a heavy chain CDR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 19, 43, 67, 91, 115, 139, 163, 187, or 211; and/or a heavy chain CDR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 20, 44, 68, 92, 116, 140, 164, 188, or 212.

In one embodiment, a humanized anti-filovirus antibody or antigen-binding portion thereof further comprises a light chain FR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 333, 341, 349, 357, 365, 373, 381, 389, or 397; a light chain FR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 334, 342, 350, 358, 366, 374, 382, 390 or 398; a light chain FR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 335, 343, 351, 359, 367, 375, 383, 391 or 399; a light chain FR4 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 336, 344, 352, 360, 368, 376, 384, 392, or 400; a heavy chain FR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NOs: 337, 345, 353, 361, 369, 377, 385, 393, or 401; a heavy chain FR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 338, 346, 354, 362, 370, 378, 386, 394, or 402; a heavy chain FR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 339, 347, 355, 363, 371, 379, 387, 395 or 403; and/or a heavy chain FIR4 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence of SEQ ID NO: 340, 348, 356, 364, 372, 380, 388, 396, or 404.

In some embodiments, a humanized anti-Filovirus antibody or antigen-binding portion thereof comprises: a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 6, 7 and 8, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 18, 19 and 20, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 333, 334, 335 and 336, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 337, 338, 339, and 340, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 30, 31, and 32, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 42, 43, and 44, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence e comprising SEQ ID NOs: 341, 342, 343, and 344, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 345, 346, 347, and 348, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ NOs: 54, 55, and 56, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ NOs: 66, 67, and 68, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 349, 350, 351, and 352, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 353, 354, 355, and 356, respectively.

In another embodiment, a humanized anti-Filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 78, 79 and 80, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 90, 91 and 92, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 357, 358, 359 and 360, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 361, 362, 363, and 364, respectively.

In another embodiment, a humanized anti-Filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs:102, 103, and 104, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 114, 115, and 116, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 365, 366, 367, and 368, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ NOs: 369, 370, 371, and 372, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 126, 127, and 128, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 138, 139, and 140, respectively; a light chain FR1, FR2, FR3, and FR4 comprising an ammo acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 373, 374, 375, and 376, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ IID NOs: 377, 378, 379, and 380, respectively;

In another embodiment, the humanized anti-lilovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 150, 151, and 152, respectively; a heavy chain CDR1, CDR2, and CDR3 comprising an amino acid sequence that has at least about: 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 162, 163, and 164, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 381, 382, 383, and 384, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 385, 386, 387 and 388, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 174, 175, and 176, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 186, 187, and 188, respectively; a light chain FR1, FR2, FR3, and FR4 comprising an amino acid sequence that has at least. about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 389, 390, 391, and 392, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 393, 394, 395, and 396, respectively.

In another embodiment, a humanized anti-filoviras antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 198, 199 and 200, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 210, 211, and 212, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 397, 398, 399, and 400, respectively; and a heavy chain FR1, FR2, FR3, and FR4 comprising an amino acid sequence that has at least about: 85%, 86%, 87%, 88%, 88%, 89%, 90%, 91%, 92%, 93%, 94%, 95%, 96%, 97%, 98%, 99%, or 100% sequence identity to an amino acid sequence comprising SEQ ID NOs: 401, 402, 403 and 404, respectively.

In another embodiment, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 14, 38, 62, 86, 110, 134, 158, 182, or 206; and/or a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 2, 26, 50, 74, 98, 12.2, 146, 170 or 194.

A humanized anti-filovirus antibody or antigen-binding portion thereof can comprise a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 14 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 2.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 38 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 26.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 62 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 50.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 86 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 74.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 110 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 98.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 134 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 122.

In some embodiments, a humanized anti-filovints antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 158 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 146.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 182 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 170.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 206 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to SEQ ID NO: 194.

In one embodiment, a humanized anti-filovirus antibody or antigen-binding portion thereof comprises a light chain CDR1 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 3, 27, 51, 75, 99, 123, 147, 171 or 195; a light: chain CDR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 4, 28, 52, 76, 100. 124, 148. 172, or 196; a light chain CDR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 5, 29, 53, 77, 101, 125, 149, 173, or 197; a heavy chain CDR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 15, 39, 63, 87, 111, 135, 159, 183, or 207; a heavy chain CDR2 comprising an ammo acid sequence comprising a sequence that has at least about 85%, 86%, 87%, 88%, 88%, 89%, 90%, 91%, 92%, 93%, 94%, 9.5%, 96%, 97%, 98%, 99%, or 100% sequence identity to an amino acid sequence encoded by a nucleotide sequence of SEQ ID NO: 16, 40, 64, 88, 112, 136, 160, 184, or 208; and/or a heavy chain CDR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 17, 41, 65, 89, 113, 137, 161, 185 or 209.

In one embodiment, a humanized anti-filovirus antibody or antigen-binding portion thereof further comprises a light chain FR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 9, 33, 57, 81, 105, 129, 153, 177, or 201; a light chain FR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 10, 34, 58, 82, 106, 130, 154, 178, or 202; a light chain FR3 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 11, 35, 59, 83, 107 131, 155, 179 or 203; a light chain FR4 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 12, 36, 60, 84, 108, 132, 156, 180 or 204; a heavy chain FR1 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 21, 45, 69, 93, 117, 141, 165, 189, or 213; a heavy chain FR2 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 22, 46, 70, 94, 118, 142, 166, 190 or 214; a heavy chain FR3 comprising an amino acid sequence comprising a sequence that has at least about. 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 23, 47, 71, 95, 119, 143, 167, 191 or 215; and/or a heavy chain FR4 comprising an amino acid sequence comprising a sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 24, 48, 72, 96, 120, 144, 168, 192 or 216.

In some embodiments, a humanized anti-fflovirus antibody or antigen-binding portion thereof comprises: a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 3, 4, and 5, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 15, 16, and 17, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%,97%, 98%, 99%, or 100% sequence identity to an amino acid sequence encoded by a nucleotide sequence of SEQ ID NOs: 9, 10, 11, and 12, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 21, 22, 23, and 24, respectively.

In another embodiment, a humanized anti-filovirus antibody comprises a light chain CUR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ NOs: 27, 28, and 29, respectively; a heavy chain CDR1, CDR2, and CDR3 comprising an amino acid sequence that: has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 39, 40, and 41, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 33, 34, 35, and 36, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 45, 46, 47 and 48, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 51, 52, and 53, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 63, 64, and 65, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 57, 58, 59 and 60, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 69, 70, 71, and 72, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ NOs: 75, 76, and 77, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ NOs: 87, 88, and 89, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ NOs: 81, 82, 83, and 84, respectively; and a heavy chain FR1, FR2, FR3, and FR4 comprising an amino acid sequence that has at least about 85%, 86%, 87%, 88%, 88%, 89%, 90%, 91% 92%, 93%, 94%, 95%, 96%, 97%, 98%, 99%, or 100% sequence identity to an amino acid sequence encoded by a nucleotide sequence of SEQ ID NOs: 93, 94, 95, and 96, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 99, 100, and 101, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs:111, 112, and 113, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 105, 106, 107, and 108, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 117, 118, 119, and 120, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 123, 124 and 125respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 135, 136, and 137, respectively; a light chain FR1, FR2, FR3, and FR4 comprising an amino.acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 129, 130, 131, and 132, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence SEQ ID NOs: 141, 142. 143, and 144, respectively.

In another embodiment, the humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ lD NOs: 147, 148 and 149, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 159, 160, and 161, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ iD NOs: 153, 154, 155, and 156, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 165, 166, 167, and 168, respectively.

In another embodiment, a humanized anti-filovirus antibody comprises a light chain. CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 171, 172, and 173, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 183, 184, and 185, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ NOs: 177, 178, 179 and 180, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 189, 190, 191, and 192, respectively.

In another embodiment, a humanized anti-filovirus antibody comprises a light chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 195, 196 and 197, respectively; a heavy chain CDR1, CDR2, and CDR3 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 207, 208, and 209, respectively; a light chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 201, 202, 203 and 204, respectively; and a heavy chain FR1, FR2, FR3, and FR4 comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NOs: 213, 214, 215 and 216, respectively.

In another embodiment, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid. sequence encoded by a nucleotide sequence of SEQ ID NO: 13, 37, 61, 85, 109, 133, 157, 181, or 205; and/or a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 1, 25, 49, 73, 97, 121, 145, 169, or 193.

A humanized anti-filovirus antibody or antigen-binding portion thereof can comprise a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 13 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 1.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 37 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 25.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 61 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 49.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 85 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 73.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%,93%, 94%, 95%, 96%, 97%, 98%, 99%, or 100% sequence identity to an amino acid sequence encoded by a nucleotide sequence of SEQ ID NO: 109 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 97.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 133 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 121.

In some embodiments, a humanized anti-Filovirus antibody or antigen-binding portion thereof comprises a VH comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 157 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 145.

In some embodiments, a humanized anti-fflovirus antibody or antigen-binding portion thereof comprises a VII comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 181 and a VL comprising an amino acid sequence that has at least about 85%, 86%, 87%, 88%, 88%, 89%, 90%, 91%, 92%, 93%, 94%, 95%, 96%, 97%, 98%, 99%, or 100% sequence identity to an amino acid sequence encoded by a nucleotide sequence of SEQ ID NO: 169.

In some embodiments, a humanized anti-filovirus antibody or antigen-binding portion thereof comprises a VH comprising an amino acid sequence that has at least about 85%, 86%, 87%, 88%, 88%, 89%, 90%, 91%, 92%, 93%, 94%, 9.5%, 96%, 97%, 98%, 99%, or 100% sequence identity to an amino acid sequence encoded by a nucleotide sequence of SEQ ID NO: 205 and a VI. comprising an amino acid sequence that has at least about 85%, 86%, 87%, 88%, 88%, 89%, 90%, 91%, 92%, 93%, 94%, 95%, 96%, 9:7%, 98%, 99%, or 100% sequence identity to an amino acid sequence encoded by a nucleotide sequence of SEQ ID NO: 193.

Another aspect of the present. disclosure is a mtiltispecific molecule comprising a humanized anti-Filovirus antibody or antigen-binding portion thereof disclosed herein. A multispecific molecule has specificity for at least two different antigens or epitopes. While such a molecule generally binds to two antigens (i.e., bispecific molecule or antibody), the term “multispecilic molecule” in the present invention encompasses an anti-filovirus antibody having specificity for two or more (such. as three) antigens. A multispecific molecule comprising an anti-filovirus antibody or antigen-binding portion thereof binds at least one epitope of a filovirus. In some embodiments, the two or more antigens are filovirus antigens. In some embodiments, the two or more antigens include an antigen that is not a filovirus antigen.

A multispecific molecule may comprise a full length antibody or a fragment of such an antibody. In one embodiment, the anti-filovirus antibody is a scFv dimer or diabody rather than whole antibody. Diabodies and scFv can be constructed without an Fc region, using only variable domains. Diabodies are bivalent, bispecific antibodies in which V_{H} and V_{L} domains are expressed on a single polypeptide chain, but using a linker that is too short to allow tbr pairing between the two domains on the same chain, thereby forcing the domains to pair with complementary domains of another chain and creating two antigen binding sites.

In one embodiment, the multivalent or multispecific anti-filovirus antibody comprises two dimerized single chain polypeptides. For instance, in one embodiment, each single chain polypeptide comprises, from amino to carboxyl terminus, a first binding domain (e.g., say), an immunoglobulin hinge region, an immunoglobulin constant region (with or without a CH1 region), a c-terminus linker, and a second binding domain. The c-terminus linker may comprise, for instance, an amino acid linker derived from an amino acid sequence of an immunoglobulin hinge region (e.g, an immunoglobulin “core” hinge region) or an amino acid sequence derived from a stalk region of a type II C lectin (e.g., NKG2A, NKG2D). In one embodiment, the c-terminus linker comprises an amino acid sequence such as (A₄S)³ or (G₄S)³. The single chain polypeptide may also comprise a heterodimerization domain so that each single chain polypeptide dirnerizes with a different single chain polypeptide such that a heterodimer is formed with up to four different binding domains.

The disclosure also includes nucleic acids (e.g., DNA or RNA) encoding an anti-filovirus antibody or antigen-binding portion thereof described herein. Nucleic acids of the disclosure include nucleic acids comprising a region that is substantially identical to a polynucleotide as listed in Table 2. Nucleic acids of the disclosure also include complementary nucleic acids. The nucleic acid sequences provided herein can be exploited using codon optimization, degenerate sequence, silent mutations, and other DNA techniques to optimize expression in a particular host, and the present disclosure encompasses such sequence modifications.

Nucleic acids encoding an anti-filovirus antibody or antigen-binding portion thereof described herein can be propagated by placing the nucleic acids in a vector. The choice of appropriate vector is well within the skill of the art. Many such vectors are available commercially. A nucleic acid encoding an antibody or antigen-binding portion thereof disclosed herein, a nucleic acid molecule encoding the polypeptide, operably linked to regulatory sequences that control transcriptional expression in an expression vector, is introduced into a host cell. In addition to transcriptional regulatory sequences, such as promoters and enhancers, expression vectors can include translational regulatory sequences and a marker gene which is suitable for selection of cells that carry the expression vector. The gene product encoded by a polynucleotide of the disclosure is expressed in any convenient expression system, including, for example, bacterial, yeast, insect, amphibian and mammalian systems.

The disclosure includes an expression vector comprising a nucleic acid segment, wherein the nucleic acid segment may comprise a nucleotide sequence with the following sequences: (a) SEQ ID NOs: 3, 4, 5, 9, 10, 11, 12, 15, 16, 17, 21, 22, 23, and 24; (b) SEQ ID NOs: 27, 28, 29, 33, 34, 35, 36, 39, 40, 41, 45, 46, 47, and 48; (c) SEQ ID NOs: 51, 52, 53, 57, 58, 59, 60, 63, 64, 65, 69, 70, 71, and 72; (d) SEQ 117 NOs: 75, 76, 77, 81, 82, 83, 84, 87, 88, 89, 93, 94, 95, and 96; (e) SEQ ID NOs: 99, 100, 101, 105, 106, 107, 108, 111, 112, 113, 117, 118, 119, and 120; (f) SEQ ID NOs: 123, 124, 125, 129, 130, 131, 132, 135, 136, 137, 141, 142, 143, and 144; (g) SEQ ID NOs: 147, 148, 149, 153, 154, 155, 156, 159, 160, 161, 165, 166, 167, and 168; (h) SEQ ID NOs: 171, 172, 173, 177, 178, 179, 180, 183, 184, 185, 189, 190, 191, and 192; (i) SEQ ID NOs: 195, 196, 197, 201, 202, 203, 204, 207, 208, 209, 213, 214, 215 and 216; a nucleotide sequence that has at least about 70%, 75%, 80%, 85%, 86%, 87%, 88%, 88%, 89%, 90%, 91%, 92%, 93%, 94%, 95%, 96%, 97%, 98%, 99%, or 100% sequence identity to one of the nucleotide sequences of (a)-(i); or a nucleotide sequence encoding the same amino acids as one of the nucleotide sequences of (a)-(i).

In some embodiments, an expression vector comprises a nucleotide sequence with the following sequences: (a) SEQ ID NOs: 1 and 13; (b) SEQ ID NOs: 25 and 37; (c) SEQ ID NOs: 49 and 61; (d) SEQ ID NOs: 73 and 85; (e) SEQ ID NOs: 97 and 109; (I) SEQ NOs: 121 and 133; (g) SEQ ID NOs: 145 and 157; (h) SEQ ID NOs: 169 and 181; (1) SEQ ID NOs: 193 and 205; a nucleotide sequence that has at least about. 70%, 75%, 80%, 85%, 86%, 87%, 88%, 88%, 89%, 90%, 91%, 92%, 93%, 94%, 95%, 96%, 97%, 98%, 99%, or 100% sequence identity to one of the nucleotide sequences of (a)-(i); or a nucleotide sequence encoding the same amino acids as one of the nucleotide sequences of (a)-(i),

Also provided herein is a host cell comprising an expression vector disclosed herein. Accordingly, antibodies and antigen-binding portions thereof disclosed herein can he produced in genetically engineered host cells according to conventional techniques. Suitable host cells are those cell types that can be transformed or transfectcd with exogenous DNA. and grown in culture, and include bacterial, eukaryotic or mammalian cells, such as cultured higher eukaryotic cells (including cultured cells of multicellular organisms), particularly cultured mammalian cells. Cultured mammalian cells are suitable hosts for production of recombinant proteins for use within the present disclosure. Examples of suitable mammalian host cells include COS-1, COS-7, HEK293, BHK21, CHO, BSC-1, HepG2, SP2/0, HeLa, myeloma or lymphoma cell. Other mammalian host cells include African green monkey kidney cells (Vera; ATCC CRL 1587), human embryonic kidney cells (293-HEK; ATCC CRL 1573), baby hamster kidney cells (BHK-21, BEIK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, Utah); see also, e.g., Chasin et al., Som. Cell. Molec. Gene. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed. monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va.

The present disclosure also provides a composition comprising an anti-filovirus antibody or antigen-binding portion thereof and one or more other antibodies or antigen-binding portions thereof, wherein the one or more other antibodies or antigen-binding portions thereof binds a protein produced by a virus in the Filoviridae family. The one or more other antibodies or antigen-binding portions thereof can bind a glycoprotein, such as GP (e.g., GP1 or GP2). In some embodiments, the one or more other antibodies or antigen-binding portions thereof binds Ebolavirus and/or Marburgvirus, such as Zaire ebolavirus, Sudan ebolavirus, Reston ebolavirus, Tai Forest ebolavirus, Bundibugyo ebolavirus, Cote d'Ivoire ebolavirus. Marburg virus or Ravn. virus, In some embodiments, the composition further comprises a pharmaceutically acceptable carrier.

In some embodiments, a composition comprising an anti-filovirus antibody or antigen-binding portion thereof disclosed herein with another antibody or antigen-binding portion thereof act synergistically when administered to a subject in need. As used herein, “synergy” or a “synergistic” response refers to an activity or improvement (e.g., prevents infection with a filovirus, prevents disease associated with a filovirus infection, reduces the number and/or severity of symptoms of a filovirus infection, stops or limits the spread of a filovirus, and/or shortens the duration of a filovirus infection at a rate) that is greater than the sum of the effect of each therapy as a monotherapy. As can be appreciated by a skilled artisan, synergy can be shown in vitro, ex vivo and in vivo.

As will be appreciated by one of skill in the art, the antibody or antigen-binding portion thereof may be used in the preparation of a medicament or pharmaceutical composition for administration (either therapeutic or prophylactic) to a subject in need of such treatment. In these embodiments, the medicament or pharmaceutical composition is prepared by mixing the antibody or antigen-binding portion thereof with a pharmaceutically acceptable carrier. The resulting composition is pharmacologically acceptable if its administration can be tolerated by a recipient patient. Accordingly, another aspect of the present disclosure is a pharmaceutical composition comprising an anti-filovirus antibody or antigen-binding portion thereof disclosed herein and a pharmaceutically acceptable carrier. The pharmaceutical composition can comprise a pharmaceutically acceptable carrier, diluent, excipient, and/or other additives, such as water, a pharmaceutical acceptable organic solvent, collagen, polyvinyl alcohol, polyvirtylpyrrolid.one, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (FBA), mannitol, sorbitol, lactose, a pharmaceutically acceptable surfactant and the like. Additives used are chosen from, but not limited to, the above or combinations thereof, as appropriate, depending on the dosage form of the present invention.

Formulation of the composition will vary according to the route of administration selected (e.g., solution, emulsion). An appropriate composition comprising the active agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form should be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancers include for example, salicylate, glycocholatellinoleate, glycholate, aprotinin, bacitracin, SDS, caprate and the like. See. e.g., Fix (J. Pharm. Sci., 85:1282-1285, 1996) and Oliyai and Stella (Ann. Rev. Pharmacol. Toxicol 32:521-544, 1993).

In some embodiments, the present disclosure provides methods for reducing, preventing, or treating a filovirus infection in a. subject in need thereof. In some embodiments, a subject in need thereof includes a subject that has been infected with a filovirus, is showing symptoms consistent with a filovirus infection, is exhibiting a filovirus infection, is suspected of having a filovirus infection, or is at risk of developing a filovirus infection. Thus, in some embodiments, provided herein is a method of treating a filovirus infection or outbreak comprising administering a therapeutically or prophylactically effective amount of an anti-filovirus antibody or antigen-binding portion thereof to an individual in need of such treatment. In some embodiments, the filovirus infection or outbreak is a Marburgvirus infection. In other embodiments, the infection is an Ebolavirus infection.

In one aspect, the antibodies or antigen-binding portion thereof may be formulated into a pharmaceutical product for providing treatment for individuals for filovirus infection, comprising a therapeutically effective amount of said antibody or antigen-binding portion. In some embodiments, an effective amount of the antibody or antigen-binding portion thereof may be formulated into a pharmaceutical product for treating an individual who has been infected with or exposed to a filovirus, who is at risk of a filovirus infection, or who is displaying symptoms of a filovirus infection (e.g., a Marburgvirus or Ebolavirus infection). A therapeutically effective amount can be determined by the skilled person. The therapeutically effective dosage of the pharmaceutical composition can be determined readily by the skilled artisan, for example, from animal studies. In addition, human clinical studies can be performed to determine the preferred effective dose for humans by a skilled artisan, The precise dose to be employed will also depend on the route of administration.

In some embodiments, the antibodies and antigen-binding portions provided herein may be administered via enteral (including without limitation oral administration and rectal administration) or parenteral (including without limitation intravenous administration, intramuscular administration, and. aerosol delivery) administration. Additional exemplary appropriate methods for administration of the antibodies and antigen-binding fragments provided herein include nasal, buccal, vaginal, ophthalmic, subcutaneous, intraperitoneal, intraarterial, spinal, intrathecal, intra-articular, intra-arterial, sub-arachnoid, sublingual, oral mucosal, bronchial, lymphatic, intra-uterine, integrated on an implantable device such as a suture or in an implantable device such as an implantable polymer, intradural, intracortical, or dermal. Such compositions would normally be administered as pharmaceutically acceptable compositions as described herein. In some embodiments, the antibodies or antigen-binding portions thereof may be administered to the subject once per day, or in multiple doses per day. In one embodiment, the antibodies or antigen-binding portions thereof are administered to the subject until symptoms improve or resolve and/or until the subject is no longer at risk of a filoviras infection.

As used herein, the term “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, duCks, geese, and the like are also non-limiting examples. The terms “mammals” and “animals” are included in this definition Both adult and newborn individuals are intended to be covered. In particular, the methods and compositions provided herein are methods and compositions for treating filovirus infections in human subjects.

In general, it is desirable to provide the recipient with a dosage of antibody which is in the range of from about 1 μg/kg body of individual to 1 g/kg body weight. It is of note that many factors are involved in determining what is a therapeutically effective dose or effective amount such as, for example but by no means limited to, the patient's age, weight, sex and general condition. Effective amounts may also vary according to the quality of the preparation and the severity of the infection or outbreak. Accordingly, it is noted that one of skill in the art will be able to determine what constitutes an “effective amount” based on a particular set of circumstances without undue experimentation.

In some embodiments, the antibody or antigen-binding portion thereof is “protective” or “neutralizing” and accordingly on administration will hinder the spread of the virus. In some embodiments, the antibodies and antigen-binding portions thereof provided herein interfere either with viral attachment, entry and/or unpackaging once inside the cell. Accordingly, in some embodiments, administering an effective amount to an individual in need of such treatment will result in at least one of the following: reduced viral load, reduction in severity of symptoms associated with the Filovirus infection, and reduced or slowed viral reproduction.

In other embodiments of the invention, the antibody or antigen-binding portion thereof described herein may be used. in a method for detecting a filovirus (e.g., Marburgvints or Ebolavirus) in a sample suspected of containing the filovirus. In other embodiments, the antibody or antigen-binding portion thereof described herein may be used in a method for diagnosing a filovirus infection. Such methods are well known in the art and a wide variety of suitable methods will he readily apparent to one of skill in the art. Such methods may involve contacting the sample to be investigated with the antibody or antigen-binding fragment thereof under conditions suitable for binding, and then detecting the bound antibody or fragment. The sample may be, for example, a biological sample, such as cells, tissue, biological fluid or the like or may be an environmental sample such as a soil or water sample or a food sample such as canned goods, meats and the like. Other suitable samples will be readily apparent to one of skill in the art.

As will be appreciated by one of skill in the art, detection antibodies must show high specificity and avidity for their antigenic target. As such, showing that a monoclonal antibody or antigen-binding fragment thereof reacts with the antigenic target derived from a highly purified or in vitro prepared sample does not guarantee that the antibody has sufficient specificity for use with biological sample. That is, the antibody must have sufficient specificity that it will not produce false positives or react with antigens from related, viruses. Examples of suitable tests for determining utility as a diagnostic or as a neutralizing mAb include but are by no means limited to negative neutralization. andior negative detection of a non-filovirus, or C-ELISA data showing competition of binding with the mouse mAbs that is being detected thereby showing that the mAbs can be used to show that an immune response to filovirus has occurred in patientlanimal sera, meaning that they were exposed/infected (abrogation of binding by human antibodies). Alternatively, biological material such as blood, mucus or stool with could be spiked or enriched. with the virus and the monoclonal antibodies used to detect added virus in the sample, which would in turn determine limits of detection as well as other parameters of the monoclonal antibodies. Biological samples from experimentally infected animals could also be used to determine the utility of the mAbs at different stages of the infection cycle.

In some embodiments, at least one of the detection antibodies is mixed with a biological sample under suitable conditions to promote binding of the at least one detection antibody with the antigenic target if the antigenic target is present in the biological sample. Binding of the detection antibody to an antigenic target within the sample is then detected using means known in the art, for example, by use of a labelled secondary antibody or other means discussed herein and/or known in the art. In some embodiments, the detection antibody (e.g., an anti-filovirus antibody disclosed herein) is labelled.

While various embodiments have been described above, it should be understood that they have been presented by way of example, and not limitation. Where methods described above indicate certain events occurring in certain order, the ordering of certain events can be modified. Additionally, certain of the events may be performed concurrently in a parallel process when possible, as well as performed sequentially as described above.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

### EXAMPLES

The invention will be further clarified, by the following examples, which are intended to be purely exemplary of the invention and in no way limiting.

### Example 1

### V Gene Sequenecing

V gene sequencing for CAN30G5 was performed by first isolating RNA from the CAN30G5 parental hybridoma clonal cell line using the RNAeasy Mini Kit. A panel of specific primers for each variable gene group was used to amplify the cDNA in a single step RT-PCR reaction to generate cDNA encoding the heavy and light chain variable domains (VH and VL) (AN30G5. The cDNAs were cloned and sequenced using standard techniques. After sequencing and identification of the variable gene, subgroup specific leader primers were designed to remove potential mutations from degenerate primers in the original primer panel to ensure sequence identity of the full variable gene sequence. The cDNA sequences encoding the VL and VH of CAN30G5 are presented as SEQ ID NO: 221 and SEQ ID NO: 229, respectively, and the amino acid sequences are shown as SEQ ID NO: 222 and. SEQ ID NO: 230, respectively. The amino acid sequences of the three light chain complementarity determining regions LCDR1, LCDR2, and LCDR3 are presented as SEQ ID NO:226, SEQ ID NO: 227, and SEQ ID NO: 228, respectively, and the HCDR1, HCDR2, and HCDR3 regions are presented as SEQ ID NO: 234, SEQ ID NO: 235, and SEQ ID NO: 236, respectively.

V gene sequencing kir the CAN40G1 parental hybridoma clonal cell line follows the same procedure as described above for CAN30G5. The cDNA sequences encoding the VL and VH of CAN40G1 are presented as SEQ ID NO: 269 and SEQ ID NO: 277, respectively, and the amino acid sequences are shown as SEQ ID NO: 270 and SEQ ID NO: 278, respectively. The amino acid sequences of the three light chain complementarity determining regions LCDR1, LCDR2, and LCDR3 are presented as SEQ ID NO: 274, SEQ ID NO: 275, and SEQ ID NO: 276, respectively, and the HCDR1, HCDR2, and HCDR3 regions are presented as SEQ ID NO: 282, SEQ ID NO: 283, and SEQ ID NO: 284, respectively.

V gene sequencing for the CAN54C12 parental hybridoma clonal cell line follows the same procedure as described above for CAN30G5. The cDNA sequences encoding the VL and VH of CAN54G2 are presented as SEQ ID NO: 237 and SEQ ID NO: 245, respectively, and the amino acid sequences are shown as SEQ ID NO: 238 and SEQ ID NO: 246, respectively. The amino acid sequences of the three light chain complementarily determining regions LCDR1, LCDR2, and LCDR3 are presented as SEQ ID NO: 242, SEQ ID NO: 243, and SEQ ID NO: 244, respectively, and the HCDR1, HCDR2, and HCDR3 regions are presented as SEQ ID NO: 250, SEQ ID NO: 251, and SEQ ID NO: 252, respectively.

### Example 2

### Humanization of Marine Marburg mAb

Humanization of CAN30G5

Human germline heavy and light chain variable domains with maximum identity alignment with the murine sequences were identified in the NCBI databases for use as identify acceptor frameworks. The human germline alleles selected were IGHV3-73-01/IGHJ4-01 (VH chain) and IGKV2-30-02/GKJ2-02 (VK chain). These human genii:line alleles were identified as best matching, and used as an acceptor framework for grafting the CDRs. All 6 CDRs (SEQ NOs: 63-65 and 51-53) corresponding to heavy and light chains were inserted into the human framework regions to encode complementarity determining regions for heavy and light chains (SEQ ID NOs: 254-256 and 262-264). The cdrCAN30G5 VL and VH regions are presented as SEQ ID NOs: 49 and 50 (nucleotide and amino acid sequences of edrCAN30G5 VL) and SEQ ID NOs: 61 and 62 (nucleotide and amino acid sequences of cdrCAN30G5 VH).

Surface exposure and/or involvement in folding or interchain contacts were determined and residues were changed or maintained based on these determinations. Antibodies huCAN30G5 and rehuCAN30G5 “Hinman engineered” were generated by identifying the closest human germline allele for CAN30G5 mAbs VH and Vk, These were then designed for use as acceptor frameworks, resulting in the VH and VL sequences of huCAN30G5. These sequences are presented as SEQ ID NOs: 1 and 2 (nucleotide and amino acid sequences of huCAN30G-5 VL, respectively) and SEQ ID NOs: 13 and 14 (nucleotide and amino acid sequences of huCAN30G5 VH, respectively). The rehu.CAN9G1. mAb was further resurfaced by substitution(s) made on surface exposed amino acids to correspond to the adopted human frameworks without disruption of the CDRs. These sequences are presented as SEQ NOs: 25 and 26 (nucleotide and amino acid sequences of rehuCAN30G5 VL) and SEQ ID NOs: 37 and 38 (nucleotide and amino acid sequences of re huCA N30G5 VH).

Humanization of CAN40G1

The humanized IgG1/k versions of the CAN40G murine mAb were created as above for CAN30G5. The human germline alleles selected were IGHV1-2-02/IGHJ4-01 (VH chain) and IGKV4-1-01/IGKJ2-0 (VK chain). All 6 CDRs (SEQ ID NOs: 135-137 and 123-125) corresponding to heavy and light chains were inserted into the human framework regions to encode cAnnpleinentarity determining regions for heavy and light chains (SEQ ID NOs: 138-140 and 126-128). The cdrCAN40G1 VL and VH regions are presented as SEQ ID NOs: 121 and 122 (nucleotide and amino acid sequences of cdrCAN40G1 VL) and SEQ ID NOs: 133 and 134 (nucleotide and amino acid sequences of cdrCAN40G1 VH). The huCAN40G1 sequences are presented as SEQ ID NOs: 73 and 74 (nucleotide and amino acid sequences of huCAN40G1 VL) and. SEQ ID NOs: 85 and 86 (nucleotide and amino acid sequences of huCAN40G1 VH). The rehuCAN40G1 sequences are presented as SEQ ID NOs 97 and 98 (nucleotide and amino acid sequences of rehuCAN40G1 VL) and SEQ ID NOs 109 and 110 (nucleotide and amino acid sequences of rehuGAN40G1 VH).

Humanization of CAN54G2

The humanized IgG1/k versions of the CAN54G2 murine mAb were created as above for CAN30G5. The human germline alleles selected were IGHV1-46-01/IGHJ4-01 (VH chain) and IGKV2-30-02/IGKJ2-02 (VK chain). All 6 CDRs (SEQ ID NOs: 207-209 and 195-197) corresponding to heavy and light chains were inserted into the human framework regions to encode complementarily determining regions for heavy and light chains (SEQ ID NOs: 66-68 and 54-56). The cdrCAN54G2 VL and VH regions are presented as SEQ ID NOs: 193 and 194 (nucleotide and amino acid sequences of cdrCAN54G2 VL) and SEQ NOs: 205 and 206 (nucleotide and amino acid sequences of cdrCAN54G2 VH). The huCAN54G2 sequences are presented as SEQ ID NOs: 145 and 146 (nucleotide and amino acid sequences of huCAN54G2 VL) and SEQ ID NOs: 157 and 158 (nucleotide and amino acid sequences of huCAN54G2 VH). The rehuCAN54G2 sequences are presented as SEQ ID NOs: 169 and 170 (nucleotide and amino acid sequences of rehuCAN54G2 VL) and SEQ ID NOs: 181 and 182 (nucleotide and amino acid sequences of rehuCAN5462 VH).

### Example 3

### Transient Expression and Purification of Humanized Marburg mAbs

The VEI and VL regions for the humanized Marburg mAbs described in. Example (cdrCAN30G5, huCAN30G5, rehuCAN30G5, cdrCAN40G1, huCAN40G1, rehuCAN40G1, cdrCAN54G2, huCAN54G2, and rehtiCAN54G2) were cloned into vectors for expression as full-sized. humanized antibodies having human IgG constant regions. The VH and VL regions of the parent murine antibody (CAN30G5, CAN40G1, and CAN54G2) were also cloned into human constant region vectors for expression as mouse-human chimeric antibodies.

Humanized Marburg mA.bs were produced by transient transfections in 293F, CHO-S or CHOK1S-V cells, One day prior to transfection. 293F, CHO-S or CHOK1S-V cells were counted using a Haemocytometer in the presence of Trypan Blue, then passaged into transfection. medium (293F cells remained in FreeStyle 293 Expression medium; CHO cells were transferred into DMEM/F12 supplemented with 10% FBS and L-Glutamine) at a concentration of 6-8×10⁵ cells/ml and incubated 24 hours at 37° C., 8% CO₂ and in a shaking incubator at 100 rpm, Freestyle Max Transfection. Agent was diluted 1/16 in Optimem before being added to 312.5 μg of the appropriate DNA also diluted in Optimem. DNA/Freestyle Max. Transfection Agent mix was incubated at room temperature for 20 minutes and added to 250×10⁶ cells in FreeStyle 293 Expression Medium (no DMSO) for 293F cells or DMEIVI/F12+10% FBS+5 mM L-Glutamine that had been treated for 3 hours with 1% DMSO for CHO cells.

The culture was harvested after incubation at 37° C./5% CO₂/125 rpm in a shaking incubator by centrifuging the culture at 1455×g for 30 minutes, removing the supernatant and filtering it through a 0.22 μM bottle top filter. The supernatant was concentrated by spin cell concentrator equipped with a 50 kDa membrane to a final volume of ˜100 mL. The concentrated supernatant was purified by Protein G purification on the FPLC or by using Protein G GraviTrap columns (manual purification system). The purified sample was buffer exchanged by spin-cell concentrator equipped with a 50 kDa membrane into D-PBS and concentrated down to a final volume of 1-2 mL. The final protein concentration was determined by BCA using the Pierce BCA Kit.

### Example 4

### Screening of Humanized Marburg mAbs

An ELISA was performed to test the binding of the humanized Marburg mAbs described in Example 3 against multiple strains of Marburg GP (Musoke, Ravn or Angola) or derivatives thereof (GPe, GP ectodomain; GPeΔmuc, GPe with mucin domain removed; GPeΔmueΔtm, GPe with both mucin and transmembrane domains removed) and to determine if they are cross-reactive to various strains of Ebola virus (EBOV) GP or derivatives thereof (GPe, GPeΔmuc, GPeΔmucΔtm). The ELISA plate was coated with 200 ng/well of antigen. The wells were blocked, with 5% skim milk then probed with 60 μl serially diluted humanized mAbs starting at (17.0 ug/mL to 2.1 ug/mL). After washing plates, binding was detected with commercial goat anti-human HRP conjugate antibody. Where appropriate, development and detection were carried out with anti-murine HRP-conjugated secondary Abs at the appropriate dilution for positive controls, murine mAbs, and negative (IgG Control mAb) controls were also run. The plate was read at 405 mn after minim um 15 minutes incubation at room temperature (RT) with ABTS substrate.

Results: huCAN30G5, huCAN40G1 and huCAN54G2 were tested for binding by ELISA to MARV Ravn, MARV Angola and MARV Musoke GPe, along with the murine versions (muCAN30G5, muCAN40G1 and muCAN54G2). Table 3 lists the ELISA results in table form. The results show both murine and humanized versions of CAN40G1, and CAN54G2 bind and recognize all of the MARV GPe's. Murine and humanized versions of CAN30G5 bind and recognize only the MARV Ravn GPe.

TABLE 3
ELISA Results of anti-MARV humanized and murine mAbs vs. MARV
Glycoproteins

| Glycoprotein | | |
|---|---|---|
| (GP) | mAb @ 2 ug/mL | OD₄₀₅ @ 30 min |
| MARV Ravn | muCAN30G5 | 0.160 |
| | huCAN30G5 | 2.740 |
| | muCAN40G1 | 0.504 |
| | huCAN40G1 | 2.753 |
| | muCAN54G2 | 0.160 |
| | huCAN54G2 | 0.890 |
| | IgG Neg Control (murine) | 0.049 |
| | IgG Neg Control (human) | 0.052 |
| MARV Angola | muCAN40G1 | 0.857 |
| | huCAN40G1 | 3.825 |
| | muCAN54G2 | 0.150 |
| | huCAN54G2 | 0.502 |
| | IgG Neg Control (murine) | 0.047 |
| | lgG Neg Control (human) | 0.054 |
| MARV Musoke | muCAN40G1 | 0.536 |
| | huCAN40G1 | 2.735 |
| | muCAN54G2 | 0.185 |
| | huCAN54G2 | 0.908 |
| | IgG Neg Control (murine) | 0.049 |
| | IgG Neg Control (human) | 0.056 |

### Evample 5

### Western Blots off Humanized Marburg mAbs

A 4-12% gradient SDS-PAGE gel is run for 1.5 hours at 200 volts with a combination of MARV and EBOV GP proteins. The gel is then transferred to a nitrocellulose membrane for a minimum of 1 hour at 45 volts. The membrane is blocked overnight at 4° C. with 5% skim milk in 1× TBST. The next day the humanized Ebola mAbs (1° Ab) described in Example 3 are diluted in 2.5% skim milk in 1×TBST at concentrations ranging from 2 μf/mL to 5 μg/mL. depending on the antibody and used to probe the membrane containing the transferred proteins for 2 hours at room temperature (RT). The membranes are then washed with 1× TBST to remove unbound 1° Ab and probed with anti-human IgG-HRP (2° Ab) at a dilution of 1:4000 to 1:5000 for 1.5 hours at RT. Where appropriate, development and detection are carried out with anti-murine HRP-conjugated secondary Abs at the appropriate dilution for controls.

### Example 6

### Pseudovirus Neutralization Assay

The humanized Marburg mAbs described in Example 3 are tested for neutralization of recombinant Vesicular stomatitis virus (VSV) pseudotyped with MARV GP. VSV pseudovirions containing a GFP gene in place of the VSV G gene (VSVΔG) and. bearing the glycoprotein of MARV are generated as previously described (Takeda. A. et al. Proc Natl Acad Sci USA, 1997. 94(26): 14764-14769). Experiments are performed in triplicate with VSVΔG bearing either full-length MARV GP (VSVΔG-GP) or mucin-deleted Δ257-425 GP (VSVΔG-GPΔmuc). Pseudovirions are incubated with anti-VSV G mAb for 1 hour at RT, then incubated with 2.5, 10 or 50 μg/mL, of each humanized Marburg mAb in DMEM-10%FBS for an additional hour. Pseudovirion/mAb complexes are added to Vero cells at a multiplicity of infection (MOI) of 0.01. After 48 hours, infection was evaluated by counting GIT-expressing cells.

### Example 7

### Mouse In Vivo Protection Experiments

All procedures with infectious marburgviruses are performed in biosafety level 4 facilities under IACUC-approved protocols. One hour pre- or post-exposure, BALB/c mice are treated intraperitoneally (IP) with purified humanized Ebola mAbs described in Example 3, non-relevant IgG, or PBS alone. The mice are then challenged IP with 1000 plaque-forming units (p.f.u.) mouse-adapted MARV. Mice are monitored for clinical signs of infections for 28 days post-exposure at which point the study ends and the mice are euthanized.

What is claimed is:

1. An isolated antibody or antigen-binding portion thereof that binds to a ilovirtts, wherein the antibody or antigen-binding portion thereof comprises:
a) a light chain CDR1 comprising an amino acid sequence that has at least about 85% sequence identity to an amino acid sequence comprising SEQ ID NO: 6, 78, or 150;
b) a light chain CDR2 comprising an amino acid sequence that has at least about 85% sequence identity to an amino acid sequence comprising SEQ ID NO: 7, 79, or 151;
c) a light chain CDR3 comprising an amino acid sequence that has at least about 85% sequence identity to an amino acid sequence comprising SEQ ID NO: 8, 80 or 152;
d) a heavy chain CDR1 comprising an amino acid sequence that has at least about 85% sequence identity to an amino acid sequence comprising SEQ ID NO: 18, 90, or 162;
a heavy chain CDR2 comprising an amino acid sequence that has at least about 85% sequence identity to an amino acid sequence comprising SEQ ID NO: 19, 91, or 163;
f) a heavy chain CDR3 comprising an amino acid sequence that has at least about 85% sequence identity to an amino acid sequence comprising SEQ ID NO: 20, 92, and 164;
g) a light chain FR1 comprising an amino acid sequence that has at: least about 85% sequence identity to an amino acid sequence e comprising SEQ ID NO: 333, 349, 357, 373, or 381;
h) a light chain FR2 comprising an amino acid sequence that has at least about 85% sequence identity to an amino acid sequence comprising SEQ ID NO: 334, 342, 350, 358, 374, 382;
i) a light chain FR3 comprising an amino acid sequence that has at least about 85% sequence identity to an amino acid sequence comprising SEQ ID NO: 335, 343, 351, 359, 367, 375, 383, or 391;
j) a light chain FR4 comprising an amino acid sequence that has at least about 85% sequence identity to an amino acid sequence comprising SEQ ID NO: 336, 344, 360, 368, or 376;
k) a heavy chain FR1 comprising an amino acid sequence that has at least about 85% sequence identity to an amino acid sequence comprising SEQ ID NOs: 337, 361, 369, 377, 385, or 393;
l) a heavy chain FR2 comprising an amino acid sequence that has at least about 85% sequence identity to an amino acid sequence comprising SEQ ID NO: 338, 346, 354, 362, 378, 386, 394, or 402;
m) a heavy chain FR3 comprising an amino acid sequence that has at least about 85% sequence identity to an amino acid sequence comprising SEQ ID NO: 339, 347, 355, 363, 371, 379, 387, 395 or 403; and,
n) a heavy chain FR4 comprising an amino acid sequence that has at least about 85% sequence identity to an amino acid sequence comprising SEQ ID NO: 340 or 372.

2. The isolated antibody or antigen-binding portion thereof of claim 1, wherein the antibody or antigen-binding portion thereof comprises:
a) a light chain CDR1, CDR2, and CDR3 comprising an amino acid sequence that has at least about 85% sequence identity to an amino acid sequence comprising SEQ NOs: 6, 7 and 8, respectively; a heavy chain CDR1, CDR2, and CDR3 comprising an amino acid sequence that has at least about 85% sequence identityto an amino acid sequence comprising SEQ ID NOs: 18, 19 and 20, respectively; a light chain FR1, FR2, FR3, and FR4 comprising an amino acid sequence that has at least about 85% sequence identity to an amino acid sequence comprising SEQ ID NOs: 333, 334, 335 and 336, respectively; and a heavy chain FR1, FR2, FR3, and FR4 comprising an amino acid sequence that has at least about 85% sequence identity to an amino acid sequence comprising SEQ ID NOs: 337, 338, 339, and 340, respectively;
b) a light chain CDR1, CDR2, and CDR3 comprising an amino acid sequence that has at least about 85% sequence identity to an amino acid sequence comprising SEQ ID NOs: 30, 31, and 32, respectively; a heavy chain CDR1, CDR2, and CDR3 comprising an amino acid sequence that has at least about 85% sequence identity to an amino acid sequence comprising SEQ ID NOs: 42, 43, and 44, respectively; a light chain FR1, FR2, FR3, and FR4 comprising an amino acid sequence that has at least about 85% sequence identity to an amino acid sequence e comprising SEQ ID NOs: 341, 342, 343, and 344, respectively; and a heavy chain FR1, FR2, FR3, and ER4 comprising an amino acid sequence that has at least about 85% sequence identity to an amino acid sequence comprising SEQ ID NOs: 345, 346, 347 and 348, respectively;
c) a light chain CDR1, CDR2, and CDR3 comprising an amino acid sequence that has at least about 85% sequence identity to an amino acid sequence comprising SEQ ID NOs: 54. 55, and 56, respectively; a heavy chain CDR1, CDR2, and CDR3 comprising an amino acid sequence that has at. least about 85% sequence identity to an amino acid sequence comprising SEQ ID NOs: 66, 67, and 68, respectively; a light chain FR1, FR2, FR3, and. FR4 comprising an amino acid sequence that has at least about 85% sequence identity to an amino acid sequence comprising SEQ ID NOs: 349, 350, 351, and 352, respectively; and a heavy chain FR1, FR2, FR3, and FR4 comprising an amino acid sequence that has at least about 85% sequence identity to an amino acid sequence comprising SEQ ID NOs: 353, 354, 355, and 356, respectively;
d) a light chain CDR1, CDR2, and CDR3 comprising an amino acid sequence that has at least about 85% sequence identity to an amino acid sequence comprising SEQ ID NOs: 78, 79 and 80, respectively; a heavy chain CDR1, CDR2, and CDR3 comprising an amino acid sequence that has at least about 85% sequence identity to an amino acid sequence comprising SEQ ID NOs: 90, 91 and 92, respectively; a light chain FR1, FR2, FR3, and FR4 comprising an amino acid sequence that has at least about 85% sequence identity to an amino acid sequence comprising SEQ ID NOs: 357, 358, 359 and 360, respectively; and a heavy chain FR1, FR2, FR3, and FR4 comprising an amino acid sequence that has at least about 85% sequence identity to an amino acid sequence comprising SEQ ID NOs: 361, 362, 363, and 364, respectively;
e) a light chain CDR1, CDR2, and CDR3 comprising an amino acid sequence that has at least about 85% sequence identity to an amino acid sequence comprising SEQ ID NOs:102, 103, and 104, respectively; a heavy chain CDR1, CDR2, and CDR3 comprising an amino acid sequence that has at least about 85% sequence identity to an amino acid sequence comprising SEQ ID NOs: 114, 115, and 116, respectively; a light chain FR1, FR2, FR3, and FR4 comprising an amino acid sequence that has at least about 85% sequence identity to an amino acid sequence comprising SEQ ID NOs: 365, 366, 367, and 368, respectively; and a heavy chain FR1, FR2, FR3, and FR4 comprising an amino acid sequence that has at least about 85% sequence identity to an amino acid sequence comprising SEQ ID NOs: 369, 370, 371, and 372, respectively;
f) a light chain CDR1, CDR2, and CDR3 comprising an amino acid sequence that has at least about 85% sequence identity to an amino acid sequence comprising SEQ ID NOs: 126, 127, and 128, respectively; a heavy chain CDR1, CDR2, and CDR3 comprising an amino acid sequence that has at least about 85% sequence identity to an amino acid sequence comprising SEQ ID NOs: 138, 139, and 140, respectively; a light chain FR1, FR2, FR3, and FR4 comprising an amino acid sequence that has at least about 85% sequence identity to an amino acid sequence comprising SEQ ID NOs: 373, 374, 375, and 376, respectively; and a heavy chain FR1, FR2, FR3, and FR4 comprising an amino acid sequence that has at least about 85% sequence identity to an amino acid sequence comprising SEQ ID NOs: 377 378, 379, and 380, respectively;
g) a light chain CDR1, CDR2. and CDR3 comprising an amino acid sequence that has at least about 85% sequence identity to an amino acid sequence comprising SEQ ID NOs: 150, 151, and 152, respectively; a heavy chain CDR1, CDR2, and CDR3 comprising an amino acid sequence that has at least about 85% sequence identity to an amino acid sequence comprising SEQ ID NOs: 1.62, 163, and 164, respectively; a light chain FR1, FR2, FR3, and FR4 comprising an amino acid sequence that has at least about 85% sequence identity to an amino acid sequence comprising SEQ ID NOs: 381, 382, 383, and 384, respectively; and a heavy chain FR1, FR2, FR3, and FR4 comprising an amino acid sequence that has at least about 85% sequence identity to an amino acid sequence comprising SEQ ID NOs: 385, 386, 387 and 388, respectively;
h) a light chain CDR1, CDR2, and CDR3 comprising an amino acid sequence that has at least about 85% sequence identity to an amino acid sequence comprising SEQ ID NOs: 174, 175, and 176, respectively; a heavy chain CDR1, CDR2, and CDR3 comprising an amino acid sequence that has at least about 85% sequence identity to an amino acid sequence comprising SEQ ID NOs: 186, 187, and 188, respectively; a light chain FR1, FR2, FR3, and FR4 comprising an amino acid sequence that has at least about 85% sequence identity to an amino acid sequence comprising SEQ ID NOs: 389, 390, 391, and 392, respectively; and a heavy chain FR1, FR2, FR3, and FR4 comprising an amino acid sequence that has at least about 85% sequence identity to an amino acid sequence comprising SEQ ID NOs: 393, 394, 395, and 396, respectively; or
i) a light chain CDR1, CDR2, and CDR3 comprising an amino acid sequence that has at least about 85% sequence identity to an amino acid sequence comprising SEQ ID NOs: 198, 199 and 200, respectively; a heavy chain CDR1, CDR2, and CDR3 comprising an amino acid sequence that has at least about 85% sequence identity to an amino acid sequence comprising SEQ ID NOs: 210, 211, and 212, respectively; a light chain FR1, FR2, FR3, and FR4 comprising an amino acid sequence that has at least about 85% sequence identity to an amino acid sequence comprising SEQ ID NOs: 397, 398, 399, and 400, respectively; and a heavy chain FR1, FR2, FR3, and FR4 comprising an amino acid sequence that has at least about 85% sequence identity to an amino acid sequence comprising SEQ ID NOs: 401, 402, 403 and 404, respectively.

3. An isolated antibody or antigen-binding portion thereof that binds to a filovirus, wherein the antibody or antigen-binding portion thereof comprises:
a) a light chain CDR1 comprising an amino acid sequence that has at least about 85% sequence identity to an amino acid sequence encoded by a nucleotide sequence comprising SEQ ID NO: 3, 75, or 147;
b) a light chain CDR2 comprising an amino acid sequence that has at least about 85% sequence identity to an amino acid sequence encoded by a nucleotide sequence comprising SEQ ID NO: 4, 76, or 148;
c) a light chain CDR3 comprising an amino acid sequence that has at least about 85% sequence identity to an amino acid sequence encoded by a nucleotide sequence comprising SEQ ID NO: 5, 77, or 149;
d) a heavy chain CDR1 comprising an amino acid sequence that has at least about 85% sequence identity to an amino acid sequence encoded by a nucleotide sequence comprising SEQ ID NOs: 15, 87, or 159;
e) a heavy chain CDR2 comprising an amino acid sequence that has at least about 85% sequence identity to an amino acid sequence encoded by a nucleotide sequence comprising SEQ ID NOs: 16, 88, or 160;
f) a heavy chain CDR3 comprising an amino acid sequence that has at least about 85% sequence identity to an amino acid sequence encoded by a nucleotide sequence comprising SEQ ID NO: 17, 89, or 161;
g) a light chain FR1 comprising an amino acid sequence that has at least about 85% sequence identity to an amino acid sequence encoded by a nucleotide sequence comprising SEQ ID NO: 9, 57, 81, 129, or 153;
h) a light chain FR2 comprising an amino acid sequence that has at least about 85% sequence identity to an amino acid sequence encoded by a nucleotide sequence comprising SEQ LD NO: 10, 34, 58, 82, 130, or 154;
i) a light chain FR3 comprising an amino acid sequence that has at least about 85% sequence identity to an amino acid sequence encoded by a nucleotide sequence comprising SEQ ID NO: 11, 35, 59, 83, 107 131, 155, or 179;
j) a light chain FR4 comprising an amino acid sequence that has at least about 85% sequence identity to an amino acid sequence encoded by a nucleotide sequence comprising SEQ ID NO: 12, 36, 60, 84, 108, or 132;
k) a heavy chain FR1 comprising an ammo acid sequence that has at least about 85% sequence identity to an amino acid sequence encoded by a nucleotide sequence comprising SEQ ID NOs: 21, 93, 117, 141, 165, or 189;
l) a heavy chain FR2 comprising an amino acid sequence that has at least about 85% sequence identity to an amino acid sequence encoded by a nucleotide sequence comprising SEQ ID NO: 22, 46, 70, 94, 142, 166, 190 or 214;
m) a heavy chain FR3 comprising an amino acid sequence that has at least about 85% sequence identity to an amino acid sequence encoded by a nucleotide sequence comprising SEQ ID NOs: 23, 47, 71, 95, 119, 143, 167, 191 or 215; and,
n) a heavy chain FR4 comprising an amino acid sequence that has at least about 85% sequence identity to an amino acid sequence encoded by a nucleotide sequence comprising SEQ ID NOs: 24, 48, or 120.

4. The isolated antibody or antigen-binding portion thereof of claim 3, wherein the antibody or antigen-binding portion thereof comprises:
a) a light chain CDR CDR2, and CDR3 comprising an amino acid sequence that has at least about 85% sequence identity to an amino acid sequence encoded by a nucleotide sequence comprising SEQ ID NOs: 3, 4, and 5, respectively; a heavy chain CDR1, CDR2, and CDR3 comprising an amino acid sequence that has at least about 85% sequence identity to an amino acid sequence encoded by a nucleotide sequence comprising SEQ ID NOs: 15, 16, and 17, respectively; a light chain FR1, FR2, FR3, and FR4 comprising an amino acid sequence that has at least about 85% sequence identity to an amino acid sequence encoded by a nucleotide sequence comprising SEQ ID NOs: 9, 10, 11, and 12, respectively; and a heavy chain FR1, FR2, FR3, and FR4 comprising an amino acid sequence that has at least about 85% sequence identity to an amino acid sequence encoded by a nucleotide sequence comprising SEQ ID NOs: 21, 22, 23, and 24, respectively;
b) a light chain CDR1, CDR2, and CDR3 comprising an amino acid sequence that has at least about 85% sequence identity to an amino acid sequence encoded by a nucleotide sequence comprising SEQ ID NOs: 27, 28, and 29, respectively; a heavy chain CDR1, CDR2, and CDR3 comprising an amino acid sequence that has at least about 85% sequence identity to an amino acid sequence encoded by a nucleotide sequence comprising SEQ ID NOs: 39, 40, and 41, respectively; a light chain FR1, FR2, FR3, and FR4 comprising an amino acid sequence that has at least about 85% sequence identity to an amino acid sequence encoded by a nucleotide sequence comprising SEQ ID NOs: 33, 34, 35, and 36, respectively; and a heavy chain FR1, FR2, FR3, and FR4 comprising an amino acid sequence that has at least about 85% sequence identity to an amino acid sequence encoded by a nucleotide sequence comprising SEQ ID NOs: 45, 46, 47 and 48, respectively;
c) a light chain CDR1, CDR2, and CDR3 comprising an amino acid sequence that has at least about 85% sequence identity to an amino acid sequence encoded by a nucleotide sequence comprising SEQ ID NOs: 51, 52, and 53, respectively; a heavy chain CDR1, CDR2, and CDR3 comprising an amino acid sequence that has at least about 85% sequence identity to an amino acid sequence encoded by a nucleotide sequence comprising SEQ ID NOs: 63, 64, and 65, respectively; a light chain FR1, FR2, FR3, and FR4 comprising an amino acid sequence that has at least about 85% sequence identity to an amino acid sequence encoded by a nucleotide sequence comprising SEQ ID NOs: 57, 58, 59 and 60, respectively; and a heavy chain FR1, FR2, FR3, and FR4 comprising an amino acid sequence that has at least about 85% sequence identity to an amino acid sequence encoded by a nucleotide sequence comprising SEQ ID NOs: 69, 70, 71, and 72, respectively;
d) a light chain CDR1, CDR2, and CDR3 comprising an amino acid sequence that has at least about 85% sequence identity to an amino acid sequence encoded by a nucleotide sequence comprising SEQ ID NOs: 75, 76, and 77, respectively; a heavy chain CDR1, CDR2, and CDR3 comprising an amino acid sequence that has at least about 85% sequence identity to an amino acid sequence encoded by a nucleotide sequence comprising SEQ ID NOs: 87, 88. and 89, respectively; a light chain FR1, FR2, FR3, and FR4 comprising an amino acid sequence that has at least about 85% sequence identity to art amino acid sequence encoded by a nucleotide sequence comprising SEQ ID NOs: 81, 82, 83, and 84, respectively; and a heavy chain FR1, FR2, FR3, and FR4 comprising an amino acid sequence that has at least about 85% sequence identity to an amino acid sequence encoded by a nucleotide sequence comprising SEQ ID NOs: 93, 94, 95, and 96, respectively;
e) a light chain CDR1, CDR2, and CDR3 comprising an amino acid sequence that has at least about 85% sequence identity to an amino acid sequence encoded by a nucleotide sequence comprising SEQ ID NOs: 99, 100, and 101, respectively; a heavy chain CDR1, CDR2, and CDR3 comprising an amino acid sequence that has at least about 85% sequence identity to an amino acid sequence encoded by a nucleotide sequence comprising SEQ ID NOs: 111, 112, and 113, respectively; a light chain FR1, FR2, FR3, and FR4 comprising an amino acid sequence that has at least about 85% sequence identity to an amino acid sequence encoded by a nucleotide sequence comprising SEQ ID NOs: 105, 106, 107, and 108, respectively; and a heavy chain FR1, FR2, FR3, and FR4 comprising an amino acid sequence that has at least about 85% sequence identity to an amino acid sequence encoded by a nucleotide sequence comprising SEQ ID NOs: 117, 118, 119, and 120, respectively;
f) a light chain CDR1, CDR2, and CDR3 comprising an amino acid sequence that has at least about 85% sequence identity to an amino acid sequence encoded by a nucleotide sequence comprising SEQ NOs: 123, 124 and 125, respectively; a heavy chain CDR1, CDR2, and CDR3 comprising an amino acid sequence that has at least about 85% sequence identity to an amino acid sequence encoded by a nucleotide sequence comprising SEQ ID NOs: 135, 136, and 137, respectively; a light chain FR1, FR2, FR3, and FR4 comprising an amino acid sequence that has at least about 85% sequence identity to an amino acid sequence encoded by a nucleotide sequence comprising SEQ ID NOs; 129, 130, 131, and 132, respectively; and a heavy chain FR1, FR2, FR3, and FR4 comprising an amino acid sequence that has at least about 85% sequence identity to an amino acid sequence encoded by a nucleotide sequence comprising SEQ ID NOs: 141, 142, 143, and 144, respectively;
g) a light chain CDR1, CDR2, and CDR3 comprising an amino acid sequence that has at least about 85% sequence identity to an amino acid sequence encoded by a nucleotide sequence comprising SEQ ID NOs: 147, 148 and 149, respectively; a heavy chain CDR1, CDR2, and CDR3 comprising an amino acid sequence that has at least about 85% sequence identity to an amino acid sequence encoded by a nucleotide sequence comprising SEQ ID NOs: 159, 160, and 161, respectively; a light chain FR1, FR2, FR3, and FR4 comprising an amino acid sequence that has at least about 85% sequence identity to an amino acid sequence encoded by a nucleotide sequence comprising SEQ ID NOs: 153, 154, 155, and 156, respectively; and a heavy chain FR1, FR2, FR3, and FR4 comprising an amino acid sequence that has at least about 85% sequence identity to an amino acid sequence encoded by a nucleotide sequence comprising SEQ ID NOs: 165, 166, 167, and 168, respectively;
h) a light chain CDR1, CDR2, and CDR3 comprising an amino acid sequence that has at least about 85% sequence identity to an amino acid sequence encoded by a nucleotide sequence comprising SEQ ID NOs: 171, 172, and 173, respectively; a heavy chain CDR1, CDR2, and CDR3 comprising an amino acid sequence that has at least about 85% sequence identity to an amino acid sequence encoded by a nucleotide sequence comprising SEQ ID NOs: 183, 184, and 185, respectively; a light chain FR1, FR2, FR3, and FR4 comprising an amino acid sequence that has at least about 85% sequence identity to an amino acid sequence encoded by a nucleotide sequence comprising SEQ ID NOs: 177, 178, 179 and 180, respectively; and a heavy chain FR1, FR2, FR3, and FR4 comprising an amino acid sequence that has at least about 85% sequence identity to an amino acid sequence encoded by a nucleotide sequence comprising SEQ NOs: 189, 190, 191, and 192, respectively; or
i) a light chain CDR1, CDR2, and CDR3 comprising an amino acid sequence that has at least about 85% sequence identity to an amino acid sequence encoded by a nucleotide sequence comprising SEQ NOs: 195, 196 and 197, respectively; a heavy chain CDR1, CDR2, and CDR3 comprising an amino acid sequence that has at least about 85% sequence identity to an amino acid sequence encoded by a nucleotide sequence comprising SEQ ID NOs: 207, 208, and 209, respectively; a light chain FR1, FR2, FR3, and FR4 comprising an amino acid sequence that has at least about 85% sequence identity to an amino acid sequence encoded by a nucleotide sequence comprising SEQ ID NOs: 201, 202, 203 and 204, respectively; and a heavy chain FR1, FR2, FR3, and FR4 comprising an amino acid sequence that has at least about 85% sequence identity to an amino acid sequence encoded by a nucleotide sequence comprising SEQ ID NOs: 213, 214, 215 and 216, respectively.

5. An isolated antibody or antigen-binding portion thereof that binds to a filovirus, wherein the antibody or antigen-binding portion thereof comprises:
a) a variable heavy chain. comprising an amino acid sequence that has at least about 98% sequence identity to an amino acid sequence encoded by a nucleotide sequence comprising SEQ ID NO: 13, 37, 61, 85, 109, 133, 157, 181, or 205; and
b) a variable light chain comprising an amino acid sequence that has at least about 98% sequence identity to an amino acid sequence encoded by a nucleotide sequence comprising SEQ ID NO: 1, 25, 49, 73, 97, 121, 145, 169, or 193.

6. The antibody or antigen-binding portion thereof of claim 5, wherein the antibody of antigen-binding portion thereof comprises:
a) a variable heavy chain comprising an amino acid sequence that has at least about 98% sequence identity to an amino acid sequence encoded by a nucleotide sequence comprising SEQ ID NO: 13 and a variable light chain comprising an amino acid sequence that has at least about 98% sequence identity to an amino acid sequence encoded by a nucleotide sequence comprising SEQ ID NO: 1;
b) a variable heavy chain comprising an amino acid sequence that has at least about 98% sequence identity to an amino acid sequence encoded by a nucleotide sequence comprising SEQ ID NO: 37 and a variable light chain comprising an amino acid sequence that has at least about 98% sequence identity to an amino acid sequence encoded by a nucleotide sequence comprising SEQ ID NO: 25;
c) a variable heavy chain comprising an amino acid sequence that has at least about 98% sequence identity to an amino acid sequence encoded by a nucleotide sequence comprising SEQ ID NO: 61 and a variable light chain comprising an amino acid sequence that has at least about 98% sequence identity to an amino acid sequence encoded by a nucleotide sequence comprising SEQ ID NO: 49;
d) a variable heavy chain comprising an amino acid sequence that has.at least about 98% sequence identity to an amino acid sequence encoded by a nucleotide sequence comprising SEQ ID NO: 85 and a variable light chain comprising an amino acid sequence that has at least about 98% sequence identity to an amino acid sequence encoded by a nucleotide sequence comprising SEQ ID NO: 73;
c) a variable heavy chain comprising an amino acid sequence that has at least about 98% sequence identity to an amino acid sequence encoded by a nucleotide sequence comprising SEQ ID NO: 109 and a variable light chain comprising an amino acid sequence that has at least about 98% sequence identity to an amino acid sequence encoded by a nucleotide sequence comprising SEQ ID NO: 97;
f) a variable heavy chain comprising an amino acid sequence that has at least about 98% sequence identity to an amino acid sequence encoded by a nucleotide sequence comprising SEQ ID NO: 133 and a variable light chain comprising an amino acid sequence that has at least about 98% sequence identity to an amino acid sequence encoded by a nucleotide sequence comprising SEQ ID NO: 121;
g) a variable heavy chain comprising an amino acid sequence that has at least about 98% sequence identity to an amino acid sequence encoded by a nucleotide sequence comprising SEQ ID NO: 157 and a variable light chain comprising an amino acid sequence that has at least about 98% sequence identity to an amino acid sequence encoded by a nucleotide sequence comprising SEQ ID NO: 145;
h) a variable heavy chain comprising an amino acid sequence that has at least about 98% sequence identity to an amino acid sequence encoded by a nucleotide sequence comprising SEQ ID NO: 181 and a variable light chain comprising an amino acid sequence that has at least about 98% sequence identity to an amino acid sequence encoded by a nucleotide sequence comprising SEQ ID NO: 169; or
i) a variable heavy chain comprising an amino acid sequence that has at least about 98% sequence identity to an amino acid sequence encoded by a nucleotide sequence comprising SEQ ID NO: 205 and a variable light chain comprising an amino acid sequence that has at least. about 98% sequence identity to an amino acid sequence encoded by a nucleotide sequence comprising SEQ NO: 193.

7. An isolated antibody or antigen-binding portion thereof that binds to a lilovirus, wherein the antibody or antigen-binding portion thereof comprises:
a) a variable heavy chain comprising an amino acid sequence that has at least about 98% sequence identity to SEQ ID NO: 14, 38, 62, 86. 110, 134. 158, 182, or 206; and
b) a variable light chain comprising an amino acid sequence that has at least about 98% sequence identity to SEQ NO: 2, 26, 50, 74, 98, 122, 146, 170 or 194.

8. The isolated antibody or antigen-binding portion of claim 7, wherein the antibody or antigen-binding portion thereof comprises:
a) a variable heavy chain comprising an amino acid sequence that has at least about 98% sequence identity to SEQ ID NO: 14 and a variable light chain comprising an amino acid sequence that has at least about 98% sequence identity to SEQ ID NO: 2;
h) a variable heavy chain comprising an amino acid sequence that has at least about 98% sequence identity to SEQ ID NO: 38 and a variable light chain comprising an amino acid sequence that has at least about 98% sequence identity to SEQ ID NO: 26;
c) a variable heavy chain comprising an amino acid sequence that has at least about 98% sequence identity to SEQ ID NO: 62 and a variable light chain comprising an amino acid sequence that has at least about 98% sequence identity to SEQ ID NO: 50;
d) a variable heavy chain comprising an amino acid sequence that has at least about 98% sequence identity to SEQ ID NO: 86 and a variable light chain comprising an amino acid sequence that has at least about 98% sequence identity to SEQ ID NO: 74;
e) a variable heavy chain comprising an amino acid sequence that has at least about 98% sequence identity to SEQ ID NO: 110 and a variable light chain comprising an amino acid sequence that has at least about 98% sequence identity to SEQ ID NO: 98;
f) a variable heavy chain comprising an amino acid sequence that has at least about 98% sequence identity to SEQ ID NO: 134 and a variable light chain comprising an amino acid sequence that has at least about 98% sequence identity to SEQ ID NO: 122;
g) a variable heavy chain comprising an amino acid sequence that has at least about 98% sequence identity to SEQ ID NO: 158 and a variable light chain comprising an amino acid sequence that has at least about 98% sequence identity to SEQ ID NO: 146;
h) a variable heavy chain comprising an amino acid sequence that has at least about 98% sequence identity to SEQ ED NO: 182 and a variable light chain comprising an amino acid sequence that has at least about 98% sequence identity to SEQ ID NO: 170; or
i) a variable heavy chain comprising an amino acid sequence that has at least about 98% sequence identity to SEQ ID NO: 206 and a variable light chain comprising an amino acid sequence that has at least about 98% sequence identity to SEQ ID NO: 194.

9. The isolated antibody or antigen-binding portion of claim 8, wherein the antibody or antigen-binding portion thereof comprises:
a) a variable heavy chain comprising an amino acid sequence comprising SEQ ID NO: 14 and a varia.ble light chain comprising an amino acid sequence comprising SEQ ID NO: 2;
b) a variable heavy chain comprising an amino acid sequence comprising SEQ ID NO: 38 and a variable light chain comprising an amino acid sequence comprising SEQ ID NO: 26:
c) a variable heavy chain comprising an amino acid sequence comprising SEQ ID NO: 62 and a variable light chain comprising an amino acid sequence comprising SEQ ID NO: 50;
d) a variable heavy chain comprising an amino acid sequence comprising SEQ ID NO: 86 and a variable light chain comprising an amino acid sequence comprising SEQ ID NO: 74;
e) a variable heavy chain comprising an amino acid sequence comprising SEQ ID NO: 110 and a variable light chain comprising an amino acid sequence comprising SEQ ID NO: 98;
f) a variable heavy chain comprising an amino acid sequence comprising SEQ ID NO: 134 and a variable light chain comprising an amino acid sequence comprising SEQ ID NO: 122;
g) a variable heavy chain comprising an amino acid. sequence comprising SEQ ID NO: 158 and a variable light chain comprising an amino acid sequence comprising SEQ ID NO: 146;
h) a variable heavy chain comprising an amino acid sequence comprising SEQ ID NO: 182 and a variable light chain comprising an amino acid sequence comprising SEQ ID NO: 170; or
i) a variable heavy chain comprising an amino acid sequence comprising SEQ ID NO: 206 and a variable light chain comprising an amino acid sequence comprising SEQ ID NO: 194.

10. The isolated antibody or antigen-binding portion thereof of any one of the preceding claims, wherein th.e antibody or antigen-binding portion is selected from the group consisting of: (i) a whole immunoglobulin; (ii) an scfv; (iii) a Fab fragment; (iv) an F(ab′)2; and (v) a disulfide linked Fv.

11. The isolated antibody of antigen-binding portion thereof of any one of the preceding claims, wherein the antibody or antigen-binding portion thereof binds to the GP subunit of the Filovirus.

12. The isolated antibody or antigen-binding portion thereof of any one of the preceding claims, wherein the antibody or antigen-binding portion thereof binds to the mucin domain of the GP1 subunit of the filovirus.

13. The isolated antibody or antigen-binding portion thereof of any one of the preceding claims, wherein the antibody or antigen-binding portion thereof binds to the GP2 subunit of the filovirus.

14. The isolated antibody or antigen-binding portion thereof of any one of the preceding claims, wherein the antibody or antigen-binding portion thereof binds to the GP2 wing subunit of the filovirus.

15. The isolated antibody or antigen-binding portion thereof of any one of the preceding claims, wherein the antibody or antigen-binding portion thereof is cross-reactive to at least two filoviruses.

16. The isolated antibody of antigen-binding portion thereof of any one of the preceding claims, wherein the filovirus is Ebolavirus or Marburgvirus.

17. The isolated antibody of antigen-binding portion thereof of any one of the preceding claims, wherein the filovirus is Zaire ebolavirus, Szrdee ebolavirus, Reston ebolavirus, Tai Forest ebolavirus, Cote d'Ivoire ebolavirus or Bundibugyo ebolavirus.

18. A nucleic acid sequence encoding the antibody or antigen-binding portion thereof of any one of claims 1-17.

19. An. expression vector comprising a promoter operably linked to a nucleotide sequence of claim 18.

20. An expression vector comprising a nucleotide sequence with the following sequences:
a) SEQ ID NOs: 3, 4, 5, 9, 10, 11, 12, 15, 16, 17, 21, 22, 23, and 24;
b) SEQ ID NOs: 27, 28, 29, 33, 34, 35, 36, 39, 40, 41, 45, 46, 47, and 48;
c) SEQ ID NOs: 51, 52, 53, 57, 58, 59, 60, 63, 64, 65, 69, 70, 71, and 72;
d) SEQ ID NOs: 75, 76, 77, 81, 82, 83, 84, 87, 88, 89.93, 94, 95, and 96;
e) SEQ ID NOs: 99, 100, 101, 105, 106, 107, 108, 111, 112, 113. 117, 118, 119, and 120,
f) SEQ ID NOs: 123, 124, 125, 129, 130, 131, 132, 135, 136, 137, 141, 142, 143, and 144;
g) SEQ ID NOs: 147, 148, 149, 153, 154, 155, 156, 159, 160, 161, 165, 166, 167, and 168;
h) SEQ ID NOs: 171, 172, 173, 177, 178, 179, 180, 183, 184, 185, 189, 190, 191, and 192; or
i) SEQ NOs: 195 196, 197, 201., 202, 203, 204, 207, 208, 209, 213, 214, 215 and 216.

21. An expression vector comprising a nucleotide sequence with the following sequences:
a) SEQ ID NOs: 1 and 13;
b) SEQ ID NOs: 25 and 37;
c) SEQ ID NOs: 49 and 61;
d) SEQ ID NOs: 73 and 85;
e) SEQ ID NOs: 97 and 109;
f) SEQ ID NOs: 121 and. 133;
g) SEQ ID NOs: 145 and 157;
h) SEQ ID NOs: 169 and 181; or
i) SEQ ID NOs: 193 and 205.

22. A host cell comprising the expression vector of any one of claims 19-21.

23. The host cell of claim 22, wherein the cell is a bacterial, eukaryotic or mammalian cell,

24. The host cell of claim 22 or 23, wherein the cell is COS-1, COS-7, HEK293, BHK21, CHO, BSC-1, HepG2, SP2/0, HeLa, myeloma or lymphoma cell.

25. A method of producing an antibody or antigen -binding portion thereof that binds to a filovirus comprising:
a) ctilturing a host cell of any one of claims 22-24; and
b) recovering the antibody or antigen-binding portion thereof.

26. A pharmaceutical composition comprising the antibody or antigen-binding portion thereof of any one of claims 1-17 and a pharmaceutically acceptable carrier.

27. A composition comprising the antibody or antigen-binding portion thereof of any one of claims 1-17 and one or more other antibodies or antigen-binding portions thereof, wherein the one or more other antibodies or antigen-binding portions thereof binds a protein produced by a virus in the Filoviridae family.

28. The composition of claim 27, wherein the protein is a glycoprotein.

29. The composition of claim 27 or 28, wherein the virus is Eboiaviras or Marburgvirus.

30. The composition of claim 29, wherein the virus is Zaire ebolavirus, Sudan ebolavirus, Reston ebolavirus, Tai Forest ebolavirus, Bundibugyo ebolavirus, Cote d'Ivoire ebolavirus, Marburg virus or Ravn virus.

31. The composition of any one of claims 27-30, further comprising a pharmaceutically acceptable carrier.

32. A method for ameliorating, treating or preventing a filovirus infection in a subject in need thereof, the method comprising administering to the subject a therapeutically effective amount of the antibody or antigen-binding portion thereof of any one of claims 1-17.

33. A method ibr ameliorating, treating or preventing a filovirus infection in a subject in need thereof, the method comprising administering to the subject a therapeutically effective amount of the composition of any one of claims 26-31.

34. The method of claim 32 or 33, wherein the subject is a human.

35. A method for detecting Marburgvirus in a sample, the method comprising contacting the Sample with the antibody or antigen-binding portion thereof of any one of claims 1-13.

32. The method of claim 35, wherein the sample is a cell, tissue, or biological fluid from a subject suspected of having or at risk of a filovims infection.

33. An expression vector comprising a nucleotide sequence selected from the group consisting of SEQ ID NOs: 1, 13, 25, 37, 49, 61, 73, 85, 97, 109, 121, 133, 145, 157, 169, 181, 193, and 205.


SEQUENCE LISTING (404 sequences)

SEQ ID NO: 1 - DNA, 337 bp, Artificial Sequence
  huCAN30G5 Chain K, Variable region
gacatcgtgctgacccagtcccccctgtccctgcccgtgaccctgggccagcccgcctccatctcctgccgctcctcccagtccctggtgcactccaacggcaacacctacctgcactggtaccagcagcgccccggccagtccccccgcctgctgatctacaaggtgtccaaccgcttctccggcgtgcccgaccgcttctccggctccggctccggcaccgacttcaccctgaagatctcccgcgtggaggccgaggacgtgggcgtgtactactgctcccagtccacccacgtgccctggaccttcggcggcggcaccaaggtggagatcaagc

SEQ ID NO: 2 - protein, 112 aa, Artificial Sequence
  huCAN30G5 Chain K, variable region
DIVLTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVPWTFGGGTKVEIK

SEQ ID NO: 3 - DNA, 33 bp, Artificial Sequence
  huCAN30G5 Chain K, CDR1
cagtccctggtgcactccaacggcaacacctac

SEQ ID NO: 4 - DNA, 9 bp, Artificial Sequence
  huCAN30G5 Chain K, CDR2
aaggtgtcc

SEQ ID NO: 5 - DNA, 27 bp, Artificial Sequence
  huCAN30G5 Chain K, CDR3
tcccagtccacccacgtgccctggacc

SEQ ID NO: 6 - protein, 11 aa, Artificial Sequence
  huCAN30G5 Chain K, CDR1
QSLVHSNGNTY

SEQ ID NO: 7 - protein, 3 aa, Artificial Sequence
  huCAN30G5 Chain K, CDR2
KVS

SEQ ID NO: 8 - protein, 9 aa, Artificial Sequence
  huCAN30G5 Chain K, CDR3
SQSTHVPWT

SEQ ID NO: 9 - DNA, 78 bp, Artificial Sequence
  huCAN30G5 Chain K, FR1
gacatcgtgctgacccagtcccccctgtccctgcccgtgaccctgggccagcccgcctccatctcctgccgctcctcc

SEQ ID NO: 10 - DNA, 51 bp, Artificial Sequence
  huCAN30G5 Chain K, FR2
ctgcactggtaccagcagcgccccggccagtccccccgcctgctgatctac

SEQ ID NO: 11 - DNA, 108 bp, Artificial Sequence
  huCAN30G5 Chain K, FR3
aaccgcttctccggcgtgcccgaccgcttctccggctccggctccggcaccgacttcaccctgaagatctcccgcgtggaggccgaggacgtgggcgtgtactactgc

SEQ ID NO: 12 - DNA, 31 bp, Artificial Sequence
  huCAN30G5 Chain K, FR4
ttcggcggcggcaccaaggtggagatcaagc

SEQ ID NO: 13 - DNA, 358 bp, Artificial Sequence
  huCAN30G5 Chain H, Variable region
gaggtgcagctggtggagtccggcggcggcctggtgcagcccggcggctccctgaagctgtcctgcgccgcctccggcttcgccttcaactcctacgccatgcactgggtgcgccaggcctccggcaagggcctggagtgggtggcccgcatccgcatcaagtccggcaactacgccacctcctacgccggctccgtgaagggccgcttcaccgtgtcccgcgacgactccaagaacaccttctacctgcagatgaactccctgaagaccgaggacaccgccatgtactactgcgtgcgcgagtgggagggcgccatggactactggggccagggcaccctggtgaccgtgtcctccg

SEQ ID NO: 14 - protein, 119 aa, Artificial Sequence
  huCAN30G5 Chain H, variable region
EVQLVESGGGLVQPGGSLKLSCAASGFAFNSYAMHWVRQASGKGLEWVARIRIKSGNYATSYAGSVKGRFTVSRDDSKNTFYLQMNSLKTEDTAMYYCVREWEGAMDYWGQGTLVTVSS

SEQ ID NO: 15 - DNA, 24 bp, Artificial Sequence
  huCAN30G5 Chain H, CDR1
ggcttcgccttcaactcctacgcc

SEQ ID NO: 16 - DNA, 30 bp, Artificial Sequence
  huCAN30G5 Chain H, CDR2
atccgcatcaagtccggcaactacgccacc

SEQ ID NO: 17 - DNA, 30 bp, Artificial Sequence
  huCAN30G5 Chain H, CDR3
gtgcgcgagtgggagggcgccatggactac

SEQ ID NO: 18 - protein, 8 aa, Artificial Sequence
  huCAN30G5 Chain H, CDR1
GFAFNSYA

SEQ ID NO: 19 - protein, 10 aa, Artificial Sequence
  huCAN30G5 Chain H, CDR2
IRIKSGNYAT

SEQ ID NO: 20 - protein, 10 aa, Artificial Sequence
  huCAN30G5 Chain H, CDR3
VREWEGAMDY

SEQ ID NO: 21 - DNA, 75 bp, Artificial Sequence
  huCAN30G5 Chain H, FR1
gaggtgcagctggtggagtccggcggcggcctggtgcagcccggcggctccctgaagctgtcctgcgccgcctcc

SEQ ID NO: 22 - DNA, 51 bp, Artificial Sequence
  huCAN30G5 Chain H, FR2
atgcactgggtgcgccaggcctccggcaagggcctggagtgggtggcccgc

SEQ ID NO: 23 - DNA, 114 bp, Artificial Sequence
  huCAN30G5 Chain H, FR3
tcctacgccggctccgtgaagggccgcttcaccgtgtcccgcgacgactccaagaacaccttctacctgcagatgaactccctgaagaccgaggacaccgccatgtactactgc

SEQ ID NO: 24 - DNA, 34 bp, Artificial Sequence
  huCAN30G5 Chain H, FR4
tggggccagggcaccctggtgaccgtgtcctccg

SEQ ID NO: 25 - DNA, 336 bp, Artificial Sequence
  rehuCAN30G5 Chain K, Variable region
gacatcgtgctgacccagtcccccctgtccctgcccgtgaccctgggccagcccgcctccatctcctgccgctcctcccagtccctggtgcactccaacggcaacacctacctgcactggtacctgcagaagcccggccagtccccccgcctgctgatctacaaggtgtccaaccgcttctccggcgtgcccgaccgcttctccggctccggctccggcaccgacttcaccctgaagatctcccgcgtggaggccgaggacgtgggcgtgtacttctgctcccagtccacccacgtgccctggaccttcggcggcggcaccaagctggagatcaag

SEQ ID NO: 26 - protein, 112 aa, Artificial Sequence
  rehuCAN30G5 Chain K, variable region
DIVLTQSPLSLPVTLGQPASISCRSSQSLVHSNGNTYLHWYLQKPGQSPRLLIYKVSNRFSGVPDRFSGSGSGTDFTLKISRVEAEDVGVYFCSQSTHVPWTFGGGTKLEIK

SEQ ID NO: 27 - DNA, 33 bp, Artificial Sequence; identical to SEQ ID NO: 3
  rehuCAN30G5 Chain K, CDR1

SEQ ID NO: 28 - DNA, 9 bp, Artificial Sequence
  rehuCAN30G5 Chain K, CDR2
aaggtgtcc

SEQ ID NO: 29 - DNA, 27 bp, Artificial Sequence
  rehuCAN30G5 Chain K, CDR3
tcccagtccacccacgtgccctggacc

SEQ ID NO: 30 - protein, 11 aa, Artificial Sequence
  rehuCAN30G5 Chain K, CDR1
QSLVHSNGNTY

SEQ ID NO: 31 - protein, 3 aa, Artificial Sequence
  rehuCAN30G5 Chain K, CDR2
KVS

SEQ ID NO: 32 - protein, 9 aa, Artificial Sequence
  rehuCAN30G5 Chain K, CDR3
SQSTHVPWT

SEQ ID NO: 33 - DNA, 78 bp, Artificial Sequence; identical to SEQ ID NO: 9
  rehuCAN30G5 Chain K, FR1

SEQ ID NO: 34 - DNA, 51 bp, Artificial Sequence
  rehuCAN30G5 Chain K, FR2
ctgcactggtacctgcagaagcccggccagtccccccgcctgctgatctac

SEQ ID NO: 35 - DNA, 108 bp, Artificial Sequence
  rehuCAN30G5 Chain K, FR3
aaccgcttctccggcgtgcccgaccgcttctccggctccggctccggcaccgacttcaccctgaagatctcccgcgtggaggccgaggacgtgggcgtgtacttctgc

SEQ ID NO: 36 - DNA, 30 bp, Artificial Sequence
  rehuCAN30G5 Chain K, FR4
ttcggcggcggcaccaagctggagatcaag

SEQ ID NO: 37 - DNA, 357 bp, Artificial Sequence
  rehuCAN30G5 Chain H, Variable region
gaggtgcagctggtggagtccggcggcggcctggtgcagcccggcggctccctgaagctgtcctgcgccgcctccggcttcgccttcaactcctacgccatgcactgggtgtgccaggcctccggcaagggcctggagtgggtggcccgcatccgcatcaagtccggcaactacgccacctcctacgccggctccgtgaagggccgcttcaccgtgtcccgcgacgactccaagtccctgttctacctgcagatgaacaacctgaagaccgaggacaccgccatgtactactgcgtgcgcgagtgggagggcgccatggactactggggccagggcaccctggtgaccgtgtcctcc

SEQ ID NO: 38 - protein, 119 aa, Artificial Sequence
  rehuCAN30G5 Chain H, variable region
EVQLVESGGGLVQPGGSLKLSCAASGFAFNSYAMHWVCQASGKGLEWVARIRIKSGNYATSYAGSVKGRFTVSRDDSKSLFYLQMNNLKTEDTAMYYCVREWEGAMDYWGQGTLVTVSS

SEQ ID NO: 39 - DNA, 24 bp, Artificial Sequence
  rehuCAN30G5 Chain H, CDR1
ggcttcgccttcaactcctacgcc

SEQ ID NO: 40 - DNA, 30 bp, Artificial Sequence; identical to SEQ ID NO: 16
  rehuCAN30G5 Chain H, CDR2

SEQ ID NO: 41 - DNA, 30 bp, Artificial Sequence; identical to SEQ ID NO: 17
  rehuCAN30G5 Chain H, CDR3

SEQ ID NO: 42 - protein, 8 aa, Artificial Sequence
  rehuCAN30G5 Chain H, CDR1
GFAFNSYA

SEQ ID NO: 43 - protein, 10 aa, Artificial Sequence
  rehuCAN30G5 Chain H, CDR2
IRIKSGNYAT

SEQ ID NO: 44 - protein, 10 aa, Artificial Sequence
  rehuCAN30G5 Chain H, CDR3
VREWEGAMDY

SEQ ID NO: 45 - DNA, 75 bp, Artificial Sequence; identical to SEQ ID NO: 21
  rehuCAN30G5 Chain H, FR1

SEQ ID NO: 46 - DNA, 51 bp, Artificial Sequence
  rehuCAN30G5 Chain H, FR2
atgcactgggtgtgccaggcctccggcaagggcctggagtgggtggcccgc

SEQ ID NO: 47 - DNA, 114 bp, Artificial Sequence
  rehuCAN30G5 Chain H, FR3
tcctacgccggctccgtgaagggccgcttcaccgtgtcccgcgacgactccaagtccctgttctacctgcagatgaacaacctgaagaccgaggacaccgccatgtactactgc

SEQ ID NO: 48 - DNA, 33 bp, Artificial Sequence
  rehuCAN30G5 Chain H, FR4
tggggccagggcaccctggtgaccgtgtcctcc

SEQ ID NO: 49 - DNA, 336 bp, Artificial Sequence
  cdrCAN30G5 Chain K, Variable region
gacgtggtgatgacccagtcccccctgtccctgcccgtgaccctgggccagcccgcctccatctcctgccgctcctcccagtccctggtgcactccaacggcaacacctacctgaactggttccagcagcgccccggccagtccccccgccgcctgatctacaaggtgtccaaccgcgactccggcgtgcccgaccgcttctccggctccggctccggcaccgacttcaccctgaagatctcccgcgtggaggccgaggacgtgggcgtgtactactgctcccagtccacccacgtgccctggaccttcggcggcggcaccaaggtggagatcaag

SEQ ID NO: 50 - protein, 112 aa, Artificial Sequence
  cdrCAN30G5 Chain K, variable region
DVVMTQSPLSLPVTLGQPASISCRSSQSLVHSNGNTYLNWFQQRPGQSPRRLIYKVSNRDSGVPDRFSGSGSGTDFTLKISRVEAEDVGVYYCSQSTHVPWTFGGGTKVEIK

SEQ ID NO: 51 - DNA, 33 bp, Artificial Sequence; identical to SEQ ID NO: 3
  cdrCAN30G5 Chain K, CDR1

SEQ ID NO: 52 - DNA, 9 bp, Artificial Sequence
  cdrCAN30G5 Chain K, CDR2
aaggtgtcc

SEQ ID NO: 53 - DNA, 27 bp, Artificial Sequence
  cdrCAN30G5 Chain K, CDR3
tcccagtccacccacgtgccctggacc

SEQ ID NO: 54 - protein, 11 aa, Artificial Sequence
  cdrCAN30G5 Chain K, CDR1
QSLVHSNGNTY

SEQ ID NO: 55 - protein, 3 aa, Artificial Sequence
  cdrCAN30G5 Chain K, CDR2
KVS

SEQ ID NO: 56 - protein, 9 aa, Artificial Sequence
  cdrCAN30G5 Chain K, CDR3
SQSTHVPWT

SEQ ID NO: 57 - DNA, 78 bp, Artificial Sequence
  cdrCAN30G5 Chain K, FR1
gacgtggtgatgacccagtcccccctgtccctgcccgtgaccctgggccagcccgcctccatctcctgccgctcctcc

SEQ ID NO: 58 - DNA, 51 bp, Artificial Sequence
  cdrCAN30G5 Chain K, FR2
ctgaactggttccagcagcgccccggccagtccccccgccgcctgatctac

SEQ ID NO: 59 - DNA, 108 bp, Artificial Sequence
  cdrCAN30G5 Chain K, FR3
aaccgcgactccggcgtgcccgaccgcttctccggctccggctccggcaccgacttcaccctgaagatctcccgcgtggaggccgaggacgtgggcgtgtactactgc

SEQ ID NO: 60 - DNA, 30 bp, Artificial Sequence
  cdrCAN30G5 Chain K, FR4
ttcggcggcggcaccaaggtggagatcaag

SEQ ID NO: 61 - DNA, 357 bp, Artificial Sequence
  cdrCAN30G5 Chain H, Variable region
gaggtgcagctggtggagtccggcggcggcctggtgcagcccggcggctccctgaagctgtcctgcgccgcctccggcttcgccttcaactcctacgccatgcactgggtgcgccaggcctccggcaagggcctggagtgggtgggccgcatccgcatcaagtccggcaactacgccaccgcctacgccgcctccgtgaagggccgcttcaccatctcccgcgacgactccaagaacaccgcctacctgcagatgaactccctgaagaccgaggacaccgccgtgtactactgcgtgcgcgagtgggagggcgccatggactactggggccagggcaccctggtgaccgtgtcctcc

SEQ ID NO: 62 - protein, 119 aa, Artificial Sequence
  cdrCAN30G5 Chain H, variable region
EVQLVESGGGLVQPGGSLKLSCAASGFAFNSYAMHWVRQASGKGLEWVGRIRIKSGNYATAYAASVKGRFTISRDDSKNTAYLQMNSLKTEDTAVYYCVREWEGAMDYWGQGTLVTVSS

SEQ ID NO: 63 - DNA, 24 bp, Artificial Sequence
  cdrCAN30G5 Chain H, CDR1
ggcttcgccttcaactcctacgcc

SEQ ID NO: 64 - DNA, 30 bp, Artificial Sequence; identical to SEQ ID NO: 16
  cdrCAN30G5 Chain H, CDR2

SEQ ID NO: 65 - DNA, 30 bp, Artificial Sequence; identical to SEQ ID NO: 17
  cdrCAN30G5 Chain H, CDR3

SEQ ID NO: 66 - protein, 8 aa, Artificial Sequence
  cdrCAN30G5 Chain H, CDR1
GFAFNSYA

SEQ ID NO: 67 - protein, 10 aa, Artificial Sequence
  cdrCAN30G5 Chain H, CDR2
IRIKSGNYAT

SEQ ID NO: 68 - protein, 10 aa, Artificial Sequence
  cdrCAN30G5 Chain H, CDR3
VREWEGAMDY

SEQ ID NO: 69 - DNA, 75 bp, Artificial Sequence; identical to SEQ ID NO: 21
  cdrCAN30G5 Chain H, FR1

SEQ ID NO: 70 - DNA, 51 bp, Artificial Sequence
  cdrCAN30G5 Chain H, FR2
atgcactgggtgcgccaggcctccggcaagggcctggagtgggtgggccgc

SEQ ID NO: 71 - DNA, 114 bp, Artificial Sequence
  cdrCAN30G5 Chain H, FR3
gcctacgccgcctccgtgaagggccgcttcaccatctcccgcgacgactccaagaacaccgcctacctgcagatgaactccctgaagaccgaggacaccgccgtgtactactgc

SEQ ID NO: 72 - DNA, 33 bp, Artificial Sequence; identical to SEQ ID NO: 48
  cdrCAN30G5 Chain H, FR4

SEQ ID NO: 73 - DNA, 334 bp, Artificial Sequence
  huCAN40G1 Chain K, Variable region
gacatcgtgctgacccagtcccccgcctccctggccgtgtccctgggcgagcgcgccaccatctcctgcaaggcctcccagtccgtggaccacgacggcgactcctacatgaactggtaccagcagaagcccggccagccccccaagctgctgatctacgccacctccaacctggagtccggcatccccgcccgcttctccggctccggctccggcaccgacttcaccctgaccatctcctccctgcaggccgaggacgtggccacctactactgccagcagtcctacgaggtgcccctgaccttcggcgccggcaccaagctggagatcaagc

SEQ ID NO: 74 - protein, 111 aa, Artificial Sequence
  huCAN40G1 Chain K, variable region
DIVLTQSPASLAVSLGERATISCKASQSVDHDGDSYMNWYQQKPGQPPKLLIYATSNLESGIPARFSGSGSGTDFTLTISSLQAEDVATYYCQQSYEVPLTFGAGTKLEIK

SEQ ID NO: 75 - DNA, 30 bp, Artificial Sequence
  huCAN40G1 Chain K, CDR1
cagtccgtggaccacgacggcgactcctac

SEQ ID NO: 76 - DNA, 9 bp, Artificial Sequence
  huCAN40G1 Chain K, CDR2
gccacctcc

SEQ ID NO: 77 - DNA, 27 bp, Artificial Sequence
  huCAN40G1 Chain K, CDR3
cagcagtcctacgaggtgcccctgacc

SEQ ID NO: 78 - protein, 10 aa, Artificial Sequence
  huCAN40G1 Chain K, CDR1
QSVDHDGDSY

SEQ ID NO: 79 - protein, 3 aa, Artificial Sequence
  huCAN40G1 Chain K, CDR2
ATS

SEQ ID NO: 80 - protein, 9 aa, Artificial Sequence
  huCAN40G1 Chain K, CDR3
QQSYEVPLT

SEQ ID NO: 81 - DNA, 78 bp, Artificial Sequence
  huCAN40G1 Chain K, FR1
gacatcgtgctgacccagtcccccgcctccctggccgtgtccctgggcgagcgcgccaccatctcctgcaaggcctcc

SEQ ID NO: 82 - DNA, 51 bp, Artificial Sequence
  huCAN40G1 Chain K, FR2
atgaactggtaccagcagaagcccggccagccccccaagctgctgatctac

SEQ ID NO: 83 - DNA, 108 bp, Artificial Sequence
  huCAN40G1 Chain K, FR3
aacctggagtccggcatccccgcccgcttctccggctccggctccggcaccgacttcaccctgaccatctcctccctgcaggccgaggacgtggccacctactactgc

SEQ ID NO: 84 - DNA, 31 bp, Artificial Sequence
  huCAN40G1 Chain K, FR4
ttcggcgccggcaccaagctggagatcaagc

SEQ ID NO: 85 - DNA, 343 bp, Artificial Sequence
  huCAN40G1 Chain H, Variable region
gaggtgcagctgcagcagtccggccccgaggtgaagaagcccggcgcctccgtgaaggtgtcctgccgcacctccggctacaccttcaccgagtacaccatccactgggtgaagcaggcccccggcaagggcctggagtggatcggcggcatcaaccccaaccacggcggcaccctgtacaaccagaagttcaagggccgcgtgaccctgaccgtggacaagtcctcctccaccgcctacatggagctgtcccgcctgcgctccgacgacaccgccgtgtactactgcgcccgcttcacctacgactactggggccagggcaccctggtgaccgtgtcctccg

SEQ ID NO: 86 - protein, 114 aa, Artificial Sequence
  huCAN40G1 Chain H, variable region
EVQLQQSGPEVKKPGASVKVSCRTSGYTFTEYTIHWVKQAPGKGLEWIGGINPNHGGTLYNQKFKGRVTLTVDKSSSTAYMELSRLRSDDTAVYYCARFTYDYWGQGTLVTVSS

SEQ ID NO: 87 - DNA, 24 bp, Artificial Sequence
  huCAN40G1 Chain H, CDR1
ggctacaccttcaccgagtacacc

SEQ ID NO: 88 - DNA, 24 bp, Artificial Sequence
  huCAN40G1 Chain H, CDR2
atcaaccccaaccacggcggcacc

SEQ ID NO: 89 - DNA, 21 bp, Artificial Sequence
  huCAN40G1 Chain H, CDR3
gcccgcttcacctacgactac

SEQ ID NO: 90 - protein, 8 aa, Artificial Sequence
  huCAN40G1 Chain H, CDR1
GYTFTEYT

SEQ ID NO: 91 - protein, 8 aa, Artificial Sequence
  huCAN40G1 Chain H, CDR2
INPNHGGT

SEQ ID NO: 92 - protein, 7 aa, Artificial Sequence
  huCAN40G1 Chain H, CDR3
ARFTYDY

SEQ ID NO: 93 - DNA, 75 bp, Artificial Sequence
  huCAN40G1 Chain H, FR1
gaggtgcagctgcagcagtccggccccgaggtgaagaagcccggcgcctccgtgaaggtgtcctgccgcacctcc

SEQ ID NO: 94 - DNA, 51 bp, Artificial Sequence
  huCAN40G1 Chain H, FR2
atccactgggtgaagcaggcccccggcaagggcctggagtggatcggcggc

SEQ ID NO: 95 - DNA, 114 bp, Artificial Sequence
  huCAN40G1 Chain H, FR3
ctgtacaaccagaagttcaagggccgcgtgaccctgaccgtggacaagtcctcctccaccgcctacatggagctgtcccgcctgcgctccgacgacaccgccgtgtactactgc

SEQ ID NO: 96 - DNA, 34 bp, Artificial Sequence; identical to SEQ ID NO: 24
  huCAN40G1 Chain H, FR4

SEQ ID NO: 97 - DNA, 333 bp, Artificial Sequence
  rehuCAN40G1 Chain K, Variable region
gacatcgtgctgacccagtcccccgcctccctggccgtgtccctgggcgagcgcgccaccatctcctgcaaggcctcccagtccgtggaccacgacggcgactcctacatgaactggtaccagcagaagcccggccagccccccaagctgctgatctacgccacctccaacctggagtccggcatccccgcccgcttctccggctccggctccggcaccgacttcaccctgaacatctcctccgtgcaggccgaggacgtggccacctactactgccagcagtcctacgaggtgcccctgaccttcggcgccggcaccaagctggagctgaag

SEQ ID NO: 98 - protein, 111 aa, Artificial Sequence
  rehuCAN40G1 Chain K, variable region
DIVLTQSPASLAVSLGERATISCKASQSVDHDGDSYMNWYQQKPGQPPKLLIYATSNLESGIPARFSGSGSGTDFTLNISSVQAEDVATYYCQQSYEVPLTFGAGTKLELK

SEQ ID NO: 99 - DNA, 30 bp, Artificial Sequence; identical to SEQ ID NO: 75
  rehuCAN40G1 Chain K, CDR1

SEQ ID NO: 100 - DNA, 9 bp, Artificial Sequence
  rehuCAN40G1 Chain K, CDR2
gccacctcc

SEQ ID NO: 101 - DNA, 27 bp, Artificial Sequence
  rehuCAN40G1 Chain K, CDR3
cagcagtcctacgaggtgcccctgacc

SEQ ID NO: 102 - protein, 10 aa, Artificial Sequence
  rehuCAN40G1 Chain K, CDR1
QSVDHDGDSY

SEQ ID NO: 103 - protein, 3 aa, Artificial Sequence
  rehuCAN40G1 Chain K, CDR2
ATS

SEQ ID NO: 104 - protein, 9 aa, Artificial Sequence
  rehuCAN40G1 Chain K, CDR3
QQSYEVPLT

SEQ ID NO: 105 - DNA, 78 bp, Artificial Sequence; identical to SEQ ID NO: 81
  rehuCAN40G1 Chain K, FR1

SEQ ID NO: 106 - DNA, 51 bp, Artificial Sequence; identical to SEQ ID NO: 82
  rehuCAN40G1 Chain K, FR2

SEQ ID NO: 107 - DNA, 108 bp, Artificial Sequence
  rehuCAN40G1 Chain K, FR3
aacctggagtccggcatccccgcccgcttctccggctccggctccggcaccgacttcaccctgaacatctcctccgtgcaggccgaggacgtggccacctactactgc

SEQ ID NO: 108 - DNA, 30 bp, Artificial Sequence
  rehuCAN40G1 Chain K, FR4
ttcggcgccggcaccaagctggagctgaag

SEQ ID NO: 109 - DNA, 342 bp, Artificial Sequence
  rehuCAN40G1 Chain H, Variable region
gaggtgcagctgcagcagtccggccccgaggtggtgaagcccggcgcctccgtgaagatctcctgccgcacctccggctacaccttcaccgagtacaccatccactgggtgaagcaggcccccggcaagggcctggagtggatcggcggcatcaaccccaaccacggcggcaccctgtacaaccagaagttcaagggccgcgccaccctgaccgtggacaagtcctcctccaccgcctacatggagctgtcccgcctgcgctccgacgacaccgccgtgtactactgcgcccgcttcacctacgactactggggccagggcaccctgctgaccgtgtcctcc

SEQ ID NO: 110 - protein, 114 aa, Artificial Sequence
  rehuCAN40G1 Chain H, variable region
EVQLQQSGPEVVKPGASVKISCRTSGYTFTEYTIHWVKQAPGKGLEWIGGINPNHGGTLYNQKFKGRATLTVDKSSSTAYMELSRLRSDDTAVYYCARFTYDYWGQGTLLTVSS

SEQ ID NO: 111 - DNA, 24 bp, Artificial Sequence
  rehuCAN40G1 Chain H, CDR1
ggctacaccttcaccgagtacacc

SEQ ID NO: 112 - DNA, 24 bp, Artificial Sequence
  rehuCAN40G1 Chain H, CDR2
atcaaccccaaccacggcggcacc

SEQ ID NO: 113 - DNA, 21 bp, Artificial Sequence
  rehuCAN40G1 Chain H, CDR3
gcccgcttcacctacgactac

SEQ ID NO: 114 - protein, 8 aa, Artificial Sequence
  rehuCAN40G1 Chain H, CDR1
GYTFTEYT

SEQ ID NO: 115 - protein, 8 aa, Artificial Sequence
  rehuCAN40G1 Chain H, CDR2
INPNHGGT

SEQ ID NO: 116 - protein, 7 aa, Artificial Sequence
  rehuCAN40G1 Chain H, CDR3
ARFTYDY

SEQ ID NO: 117 - DNA, 75 bp, Artificial Sequence
  rehuCAN40G1 Chain H, FR1
gaggtgcagctgcagcagtccggccccgaggtggtgaagcccggcgcctccgtgaagatctcctgccgcacctcc

SEQ ID NO: 118 - DNA, 51 bp, Artificial Sequence; identical to SEQ ID NO: 94
  rehuCAN40G1 Chain H, FR2

SEQ ID NO: 119 - DNA, 114 bp, Artificial Sequence
  rehuCAN40G1 Chain H, FR3
ctgtacaaccagaagttcaagggccgcgccaccctgaccgtggacaagtcctcctccaccgcctacatggagctgtcccgcctgcgctccgacgacaccgccgtgtactactgc

SEQ ID NO: 120 - DNA, 33 bp, Artificial Sequence
  rehuCAN40G1 Chain H, FR4
tggggccagggcaccctgctgaccgtgtcctcc

SEQ ID NO: 121 - DNA, 333 bp, Artificial Sequence
  cdrCAN40G1 Chain K, Variable region
gacatcgtgatgacccagtcccccgactccctggccgtgtccctgggcgagcgcgccaccatcaactgcaagtcctcccagtccgtggaccacgacggcgactcctacctggcctggtaccagcagaagcccggccagccccccaagctgctgatctacgccacctccacccgcgagtccggcgtgcccgaccgcttctccggctccggctccggcaccgacttcaccctgaccatctcctccctgcaggccgaggacgtggccgtgtactactgccagcagtcctacgaggtgcccctgaccttcggccagggcaccaagctggagatcaag

SEQ ID NO: 122 - protein, 111 aa, Artificial Sequence
  cdrCAN40G1 Chain K, variable region
DIVMTQSPDSLAVSLGERATINCKSSQSVDHDGDSYLAWYQQKPGQPPKLLIYATSTRESGVPDRFSGSGSGTDFTLTISSLQAEDVAVYYCQQSYEVPLTFGQGTKLEIK

SEQ ID NO: 123 - DNA, 30 bp, Artificial Sequence; identical to SEQ ID NO: 75
  cdrCAN40G1 Chain K, CDR1

SEQ ID NO: 124 - DNA, 9 bp, Artificial Sequence
  cdrCAN40G1 Chain K, CDR2
gccacctcc

SEQ ID NO: 125 - DNA, 27 bp, Artificial Sequence
  cdrCAN40G1 Chain K, CDR3
cagcagtcctacgaggtgcccctgacc

SEQ ID NO: 126 - protein, 10 aa, Artificial Sequence
  cdrCAN40G1 Chain K, CDR1
QSVDHDGDSY

SEQ ID NO: 127 - protein, 3 aa, Artificial Sequence
  cdrCAN40G1 Chain K, CDR2
ATS

SEQ ID NO: 128 - protein, 9 aa, Artificial Sequence
  cdrCAN40G1 Chain K, CDR3
QQSYEVPLT

SEQ ID NO: 129 - DNA, 78 bp, Artificial Sequence
  cdrCAN40G1 Chain K, FR1
gacatcgtgatgacccagtcccccgactccctggccgtgtccctgggcgagcgcgccaccatcaactgcaagtcctcc

SEQ ID NO: 130 - DNA, 51 bp, Artificial Sequence
  cdrCAN40G1 Chain K, FR2
ctggcctggtaccagcagaagcccggccagccccccaagctgctgatctac

SEQ ID NO: 131 - DNA, 108 bp, Artificial Sequence
  cdrCAN40G1 Chain K, FR3
acccgcgagtccggcgtgcccgaccgcttctccggctccggctccggcaccgacttcaccctgaccatctcctccctgcaggccgaggacgtggccgtgtactactgc

SEQ ID NO: 132 - DNA, 30 bp, Artificial Sequence
  cdrCAN40G1 Chain K, FR4
ttcggccagggcaccaagctggagatcaag

SEQ ID NO: 133 - DNA, 342 bp, Artificial Sequence
  cdrCAN40G1 Chain H, Variable region
caggtgcagctggtgcagtccggcgccgaggtgaagaagcccggcgcctccgtgaaggtgtcctgcaaggcctccggctacaccttcaccgagtacaccatgcactgggtgcgccaggcccccggccagggcctggagtggatgggctggatcaaccccaaccacggcggcaccaactacgcccagaagttccagggccgcgtgaccatgacccgcgacacctccatctccaccgcctacatggagctgtcccgcctgcgctccgacgacaccgccgtgtactactgcgcccgcttcacctacgactactggggccagggcaccctggtgaccgtgtcctcc

SEQ ID NO: 134 - protein, 114 aa, Artificial Sequence
  cdrCAN40G1 Chain H, variable region
QVQLVQSGAEVKKPGASVKVSCKASGYTFTEYTMHWVRQAPGQGLEWMGWINPNHGGTNYAQKFQGRVTMTRDTSISTAYMELSRLRSDDTAVYYCARFTYDYWGQGTLVTVSS

SEQ ID NO: 135 - DNA, 24 bp, Artificial Sequence
  cdrCAN40G1 Chain H, CDR1
ggctacaccttcaccgagtacacc

SEQ ID NO: 136 - DNA, 24 bp, Artificial Sequence
  cdrCAN40G1 Chain H, CDR2
atcaaccccaaccacggcggcacc

SEQ ID NO: 137 - DNA, 21 bp, Artificial Sequence
  cdrCAN40G1 Chain H, CDR3
gcccgcttcacctacgactac

SEQ ID NO: 138 - protein, 8 aa, Artificial Sequence
  cdrCAN40G1 Chain H, CDR1
GYTFTEYT

SEQ ID NO: 139 - protein, 8 aa, Artificial Sequence
  cdrCAN40G1 Chain H, CDR2
INPNHGGT

SEQ ID NO: 140 - protein, 7 aa, Artificial Sequence
  cdrCAN40G1 Chain H, CDR3
ARFTYDY

SEQ ID NO: 141 - DNA, 75 bp, Artificial Sequence
  cdrCAN40G1 Chain H, FR1
caggtgcagctggtgcagtccggcgccgaggtgaagaagcccggcgcctccgtgaaggtgtcctgcaaggcctcc

SEQ ID NO: 142 - DNA, 51 bp, Artificial Sequence
  cdrCAN40G1 Chain H, FR2
atgcactgggtgcgccaggcccccggccagggcctggagtggatgggctgg

SEQ ID NO: 143 - DNA, 114 bp, Artificial Sequence
  cdrCAN40G1 Chain H, FR3
aactacgcccagaagttccagggccgcgtgaccatgacccgcgacacctccatctccaccgcctacatggagctgtcccgcctgcgctccgacgacaccgccgtgtactactgc

SEQ ID NO: 144 - DNA, 33 bp, Artificial Sequence; identical to SEQ ID NO: 48
  cdrCAN40G1 Chain H, FR4

SEQ ID NO: 145 - DNA, 337 bp, Artificial Sequence
  huCAN54G2 Chain K, Variable region
gacgtggtgatgacccagacccccctgaccctgcccgtgaccctgggccagcccgcctccatctcctgcacctcctcccagtccctgctgaactccgacggcgagacctacctgaactggctgctgcagcgccccggccagtcccccaagcgcctgatccacctggtgtccaagctggactccggcgtgcccgaccgcttctccggctccggctccggcaccgacttcaccctgaagatctcccgcgtggaggccgaggacgtgggcgtgtactactgctggcagggcacccacttcccctggaccttcggcggcggcaccaaggtggagatcaagc

SEQ ID NO: 146 - protein, 112 aa, Artificial Sequence
  huCAN54G2 Chain K, variable region
DVVMTQTPLTLPVTLGQPASISCTSSQSLLNSDGETYLNWLLQRPGQSPKRLIHLVSKLDSGVPDRFSGSGSGTDFTLKISRVEAEDVGVYYCWQGTHFPWTFGGGTKVEIK

SEQ ID NO: 147 - DNA, 33 bp, Artificial Sequence
  huCAN54G2 Chain K, CDR1
cagtccctgctgaactccgacggcgagacctac

SEQ ID NO: 148 - DNA, 9 bp, Artificial Sequence
  huCAN54G2 Chain K, CDR2
ctggtgtcc

SEQ ID NO: 149 - DNA, 27 bp, Artificial Sequence
  huCAN54G2 Chain K, CDR3
tggcagggcacccacttcccctggacc

SEQ ID NO: 150 - protein, 11 aa, Artificial Sequence
  huCAN54G2 Chain K, CDR1
QSLLNSDGETY

SEQ ID NO: 151 - protein, 3 aa, Artificial Sequence
  huCAN54G2 Chain K, CDR2
LVS

SEQ ID NO: 152 - protein, 9 aa, Artificial Sequence
  huCAN54G2 Chain K, CDR3
WQGTHFPWT

SEQ ID NO: 153 - DNA, 78 bp, Artificial Sequence
  huCAN54G2 Chain K, FR1
gacgtggtgatgacccagacccccctgaccctgcccgtgaccctgggccagcccgcctccatctcctgcacctcctcc

SEQ ID NO: 154 - DNA, 51 bp, Artificial Sequence
  huCAN54G2 Chain K, FR2
ctgaactggctgctgcagcgccccggccagtcccccaagcgcctgatccac

SEQ ID NO: 155 - DNA, 108 bp, Artificial Sequence
  huCAN54G2 Chain K, FR3
aagctggactccggcgtgcccgaccgcttctccggctccggctccggcaccgacttcaccctgaagatctcccgcgtggaggccgaggacgtgggcgtgtactactgc

SEQ ID NO: 156 - DNA, 31 bp, Artificial Sequence; identical to SEQ ID NO: 12
  huCAN54G2 Chain K, FR4

SEQ ID NO: 157 - DNA, 349 bp, Artificial Sequence
  huCAN54G2 Chain H, Variable region
caggtgcagctgcagcagtccggcaccgaggtgaagaagcccggcgcctccgtgaaggtgtcctgcaaggcctccggctacgacttcaccaacttctggctgggctggatccgccaggcccccggccagggcctggagtggatcggcgacatctaccccggcggcgacaacacctactacaacgagaagttcaagggccgcgtgaccctgaccgccgacaagtcctccaacaccgcctacatggagctgtcctccctgcgctccgaggacaccgccgtgtacttctgcttcatgatcctgtacaccctggactactggggccagggcaccctggtgaccgtgtcctccg

SEQ ID NO: 158 - protein, 116 aa, Artificial Sequence
  huCAN54G2 Chain H, variable region
QVQLQQSGTEVKKPGASVKVSCKASGYDFTNFWLGWIRQAPGQGLEWIGDIYPGGDNTYYNEKFKGRVTLTADKSSNTAYMELSSLRSEDTAVYFCFMILYTLDYWGQGTLVTVSS

SEQ ID NO: 159 - DNA, 24 bp, Artificial Sequence
  huCAN54G2 Chain H, CDR1
ggctacgacttcaccaacttctgg

SEQ ID NO: 160 - DNA, 24 bp, Artificial Sequence
  huCAN54G2 Chain H, CDR2
atctaccccggcggcgacaacacc

SEQ ID NO: 161 - DNA, 27 bp, Artificial Sequence
  huCAN54G2 Chain H, CDR3
ttcatgatcctgtacaccctggactac

SEQ ID NO: 162 - protein, 8 aa, Artificial Sequence
  huCAN54G2 Chain H, CDR1
GYDFTNFW

SEQ ID NO: 163 - protein, 8 aa, Artificial Sequence
  huCAN54G2 Chain H, CDR2
IYPGGDNT

SEQ ID NO: 164 - protein, 9 aa, Artificial Sequence
  huCAN54G2 Chain H, CDR3
FMILYTLDY

SEQ ID NO: 165 - DNA, 75 bp, Artificial Sequence
  huCAN54G2 Chain H, FR1
caggtgcagctgcagcagtccggcaccgaggtgaagaagcccggcgcctccgtgaaggtgtcctgcaaggcctcc

SEQ ID NO: 166 - DNA, 51 bp, Artificial Sequence
  huCAN54G2 Chain H, FR2
ctgggctggatccgccaggcccccggccagggcctggagtggatcggcgac

SEQ ID NO: 167 - DNA, 114 bp, Artificial Sequence
  huCAN54G2 Chain H, FR3
tactacaacgagaagttcaagggccgcgtgaccctgaccgccgacaagtcctccaacaccgcctacatggagctgtcctccctgcgctccgaggacaccgccgtgtacttctgc

SEQ ID NO: 168 - DNA, 34 bp, Artificial Sequence; identical to SEQ ID NO: 24
  huCAN54G2 Chain H, FR4

SEQ ID NO: 169 - DNA, 336 bp, Artificial Sequence
  rehuCAN54G2 Chain K, Variable region
gacgtggtgatgacccagacccccctgaccctgcccgtgaccctgggccagcccgcctccatctcctgcacctcctcccagtccctgctgaactccgacggcgagacctacctgaactggctgctgcagcgccccggccagtcccccaagcgcctgatccacctggtgtccaagctggactccggcgtgcccgaccgcatctccggctccggctccggcaccgacttcaccctgaagatctcccgcgtggaggccgaggacctgggcatctactactgctggcagggcacccacttcccctggaccttcggcggcggcaccaaggtggagatcaag

SEQ ID NO: 170 - protein, 112 aa, Artificial Sequence
  rehuCAN54G2 Chain K, variable region
DVVMTQTPLTLPVTLGQPASISCTSSQSLLNSDGETYLNWLLQRPGQSPKRLIHLVSKLDSGVPDRISGSGSGTDFTLKISRVEAEDLGIYYCWQGTHFPWTFGGGTKVEIK

SEQ ID NO: 171 - DNA, 33 bp, Artificial Sequence; identical to SEQ ID NO: 147
  rehuCAN54G2 Chain K, CDR1

SEQ ID NO: 172 - DNA, 9 bp, Artificial Sequence
  rehuCAN54G2 Chain K, CDR2
ctggtgtcc

SEQ ID NO: 173 - DNA, 27 bp, Artificial Sequence
  rehuCAN54G2 Chain K, CDR3
tggcagggcacccacttcccctggacc

SEQ ID NO: 174 - protein, 11 aa, Artificial Sequence
  rehuCAN54G2 Chain K, CDR1
QSLLNSDGETY

SEQ ID NO: 175 - protein, 3 aa, Artificial Sequence
  rehuCAN54G2 Chain K, CDR2
LVS

SEQ ID NO: 176 - protein, 9 aa, Artificial Sequence
  rehuCAN54G2 Chain K, CDR3
WQGTHFPWT

SEQ ID NO: 177 - DNA, 78 bp, Artificial Sequence; identical to SEQ ID NO: 153
  rehuCAN54G2 Chain K, FR1

SEQ ID NO: 178 - DNA, 51 bp, Artificial Sequence; identical to SEQ ID NO: 154
  rehuCAN54G2 Chain K, FR2

SEQ ID NO: 179 - DNA, 108 bp, Artificial Sequence
  rehuCAN54G2 Chain K, FR3
aagctggactccggcgtgcccgaccgcatctccggctccggctccggcaccgacttcaccctgaagatctcccgcgtggaggccgaggacctgggcatctactactgc

SEQ ID NO: 180 - DNA, 30 bp, Artificial Sequence; identical to SEQ ID NO: 60
  rehuCAN54G2 Chain K, FR4

SEQ ID NO: 181 - DNA, 348 bp, Artificial Sequence
  rehuCAN54G2 Chain H, Variable region
caggtgcagctgcagcagtccggcaccgaggtggtgaagcccggcgcctccgtgaagatctcctgcaaggcctccggctacgacttcaccaacttctggctgggctggatcaagcaggcccccggccagggcctggagtggatcggcgacatctaccccggcggcgacaacacctactacaacgagaagttcaagggcaaggtgaccctgaccgccgacaagtcctccaacaccgcctacatggagttctcctccctgcgctccgaggacaccgccgtgtacttctgcttcatgatcctgtacaccctggactactggggccagggcaccctggtgaccgtgtcctcc

SEQ ID NO: 182 - protein, 116 aa, Artificial Sequence
  rehuCAN54G2 Chain H, variable region
QVQLQQSGTEVVKPGASVKISCKASGYDFTNFWLGWIKQAPGQGLEWIGDIYPGGDNTYYNEKFKGKVTLTADKSSNTAYMEFSSLRSEDTAVYFCFMILYTLDYWGQGTLVTVSS

SEQ ID NO: 183 - DNA, 24 bp, Artificial Sequence
  rehuCAN54G2 Chain H, CDR1
ggctacgacttcaccaacttctgg

SEQ ID NO: 184 - DNA, 24 bp, Artificial Sequence
  rehuCAN54G2 Chain H, CDR2
atctaccccggcggcgacaacacc

SEQ ID NO: 185 - DNA, 27 bp, Artificial Sequence
  rehuCAN54G2 Chain H, CDR3
ttcatgatcctgtacaccctggactac

SEQ ID NO: 186 - protein, 8 aa, Artificial Sequence
  rehuCAN54G2 Chain H, CDR1
GYDFTNFW

SEQ ID NO: 187 - protein, 8 aa, Artificial Sequence
  rehuCAN54G2 Chain H, CDR2
IYPGGDNT

SEQ ID NO: 188 - protein, 9 aa, Artificial Sequence
  rehuCAN54G2 Chain H, CDR3
FMILYTLDY

SEQ ID NO: 189 - DNA, 75 bp, Artificial Sequence
  rehuCAN54G2 Chain H, FR1
caggtgcagctgcagcagtccggcaccgaggtggtgaagcccggcgcctccgtgaagatctcctgcaaggcctcc

SEQ ID NO: 190 - DNA, 51 bp, Artificial Sequence
  rehuCAN54G2 Chain H, FR2
ctgggctggatcaagcaggcccccggccagggcctggagtggatcggcgac

SEQ ID NO: 191 - DNA, 114 bp, Artificial Sequence
  rehuCAN54G2 Chain H, FR3
tactacaacgagaagttcaagggcaaggtgaccctgaccgccgacaagtcctccaacaccgcctacatggagttctcctccctgcgctccgaggacaccgccgtgtacttctgc

SEQ ID NO: 192 - DNA, 33 bp, Artificial Sequence; identical to SEQ ID NO: 48
  rehuCAN54G2 Chain H, FR4

SEQ ID NO: 193 - DNA, 336 bp, Artificial Sequence
  cdrCAN54G2 Chain K, Variable region
gacgtggtgatgacccagtcccccctgtccctgcccgtgaccctgggccagcccgcctccatctcctgccgctcctcccagtccctgctgaactccgacggcgagacctacctgaactggttccagcagcgccccggccagtccccccgccgcctgatctacctggtgtccaaccgcgactccggcgtgcccgaccgcttctccggctccggctccggcaccgacttcaccctgaagatctcccgcgtggaggccgaggacgtgggcgtgtactactgctggcagggcacccacttcccctggaccttcggcggcggcaccaaggtggagatcaag

SEQ ID NO: 194 - protein, 112 aa, Artificial Sequence
  cdrCAN54G2 Chain K, variable region
DVVMTQSPLSLPVTLGQPASISCRSSQSLLNSDGETYLNWFQQRPGQSPRRLIYLVSNRDSGVPDRFSGSGSGTDFTLKISRVEAEDVGVYYCWQGTHFPWTFGGGTKVEIK

SEQ ID NO: 195 - DNA, 33 bp, Artificial Sequence; identical to SEQ ID NO: 147
  cdrCAN54G2 Chain K, CDR1

SEQ ID NO: 196 - DNA, 9 bp, Artificial Sequence
  cdrCAN54G2 Chain K, CDR2
ctggtgtcc

SEQ ID NO: 197 - DNA, 27 bp, Artificial Sequence
  cdrCAN54G2 Chain K, CDR3
tggcagggcacccacttcccctggacc

SEQ ID NO: 198 - protein, 11 aa, Artificial Sequence
  cdrCAN54G2 Chain K, CDR1
QSLLNSDGETY

SEQ ID NO: 199 - protein, 3 aa, Artificial Sequence
  cdrCAN54G2 Chain K, CDR2
LVS

SEQ ID NO: 200 - protein, 9 aa, Artificial Sequence
  cdrCAN54G2 Chain K, CDR3
WQGTHFPWT

SEQ ID NO: 201 - DNA, 78 bp, Artificial Sequence; identical to SEQ ID NO: 57
  cdrCAN54G2 Chain K, FR1

SEQ ID NO: 202 - DNA, 51 bp, Artificial Sequence; identical to SEQ ID NO: 58
  cdrCAN54G2 Chain K, FR2

SEQ ID NO: 203 - DNA, 108 bp, Artificial Sequence; identical to SEQ ID NO: 59
  cdrCAN54G2 Chain K, FR3

SEQ ID NO: 204 - DNA, 30 bp, Artificial Sequence; identical to SEQ ID NO: 60
  cdrCAN54G2 Chain K, FR4

SEQ ID NO: 205 - DNA, 348 bp, Artificial Sequence
  cdrCAN54G2 Chain H, Variable region
caggtgcagctggtgcagtccggcgccgaggtgaagaagcccggcgcctccgtgaaggtgtcctgcaaggcctccggctacgacttcaccaacttctggatgcactgggtgcgccaggcccccggccagggcctggagtggatgggcatcatctaccccggcggcgacaacacctcctacgcccagaagttccagggccgcgtgaccatgacccgcgacacctccacctccaccgtgtacatggagctgtcctccctgcgctccgaggacaccgccgtgtactactgcttcatgatcctgtacaccctggactactggggccagggcaccctggtgaccgtgtcctcc

SEQ ID NO: 206 - protein, 116 aa, Artificial Sequence
  cdrCAN54G2 Chain H, variable region
QVQLVQSGAEVKKPGASVKVSCKASGYDFTNFWMHWVRQAPGQGLEWMGIIYPGGDNTSYAQKFQGRVTMTRDTSTSTVYMELSSLRSEDTAVYYCFMILYTLDYWGQGTLVTVSS

SEQ ID NO: 207 - DNA, 24 bp, Artificial Sequence
  cdrCAN54G2 Chain H, CDR1
ggctacgacttcaccaacttctgg

SEQ ID NO: 208 - DNA, 24 bp, Artificial Sequence
  cdrCAN54G2 Chain H, CDR2
atctaccccggcggcgacaacacc

SEQ ID NO: 209 - DNA, 27 bp, Artificial Sequence
  cdrCAN54G2 Chain H, CDR3
ttcatgatcctgtacaccctggactac

SEQ ID NO: 210 - protein, 8 aa, Artificial Sequence
  cdrCAN54G2 Chain H, CDR1
GYDFTNFW

SEQ ID NO: 211 - protein, 8 aa, Artificial Sequence
  cdrCAN54G2 Chain H, CDR2
IYPGGDNT

SEQ ID NO: 212 - protein, 9 aa, Artificial Sequence
  cdrCAN54G2 Chain H, CDR3
FMILYTLDY

SEQ ID NO: 213 - DNA, 75 bp, Artificial Sequence; identical to SEQ ID NO: 141
  cdrCAN54G2 Chain H, FR1

SEQ ID NO: 214 - DNA, 51 bp, Artificial Sequence
  cdrCAN54G2 Chain H, FR2
atgcactgggtgcgccaggcccccggccagggcctggagtggatgggcatc

SEQ ID NO: 215 - DNA, 114 bp, Artificial Sequence
  cdrCAN54G2 Chain H, FR3
tcctacgcccagaagttccagggccgcgtgaccatgacccgcgacacctccacctccaccgtgtacatggagctgtcctccctgcgctccgaggacaccgccgtgtactactgc

SEQ ID NO: 216 - DNA, 33 bp, Artificial Sequence; identical to SEQ ID NO: 48
  cdrCAN54G2 Chain H, FR4

SEQ ID NO: 217 - protein, 468 aa, Artificial Sequence
  Synthetic Marburg virus Ravn Gpe delta muc Gpe delta muc delta TM
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETQRNDTGCFGILQEYNSTNNQTCPPSLKPPSLPTVTPSIHSTNTQINTAKSGTMRPPIYFRKKRSIFWKEGDIFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEET

SEQ ID NO: 218 - protein, 468 aa, Artificial Sequence
  Synthetic Marburg virus Angola Gpe delta muc Gpe delta muc delta TM
MKTTCLLISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGT

SEQ ID NO: 219 - protein, 468 aa, Artificial Sequence
  Synthetic Marburg virus Musoke Gpe delta muc Gpe delta muc delta TM
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGT

SEQ ID NO: 220 - protein, 468 aa, Artificial Sequence
  Synthetic Marburg virus Ci67 Gpe delta muc Gpe delta muc delta TM
MKTTCLFISLILIQGIKTLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGRVFTEGNIAAMIVNKTVHKMIFSRQGQGYRHMNLTSTNKYWTSNNGTQTNDTGCFGALQEYNSTKNQTCAPSKIPSPLPTARPEIKPTSTPTDATTLNTTQHLVYFRKKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGT

SEQ ID NO: 221 - DNA, 336 bp, Mus sp.
gatattgtgctgacccaatctccactctccctgcctgtcagtcttggagatcaagcctccatctcttgcagatctagtcagagccttgtacacagtaatggaaacacctatttacattggtacctgcagaagccaggccagtctccaaacctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 222 - protein, 112 aa, Mus sp.
DIVLTQSPLSLPVSLGDQASISCRSSQSLVHSNGNTYLHWYLQKPGQSPNLLIYKVSNRFSGVPDRFSGSGSGTDFTLKISRVEAEDLGVYFCSQSTHVPWTFGGGTKLEIK

SEQ ID NO: 223 - DNA, 33 bp, Mus sp.
cagagccttgtacacagtaatggaaacacctat

SEQ ID NO: 224 - DNA, 9 bp, Mus sp.
aaagtttcc

SEQ ID NO: 225 - DNA, 27 bp, Mus sp.
tctcaaagtacacatgttccgtggacg

SEQ ID NO: 226 - protein, 11 aa, Mus sp.
QSLVHSNGNTY

SEQ ID NO: 227 - protein, 3 aa, Mus sp.
KVS

SEQ ID NO: 228 - protein, 9 aa, Mus sp.
SQSTHVPWT

SEQ ID NO: 229 - DNA, 358 bp, Mus sp.
gaggtgcagcttgttgagtctggtggaggattggtgcagcctaaaggatcattgaaactctcatgtgccgcctctggtttcgccttcaattcctatgccatgcactgggtctgccaggctccaggaaagggtttggaatgggttgctcgcataagaattaaaagtggtaattatgcaacatcttatgccggttcagtgacagacagattcaccgtctccagagatgattcacaaaacttgttctatctgcaaatgaacaacctgaaaactgaggacacagccatgtattactgtgtgagagagtgggaaggggctatggactactggggtcaaggaacctcagtcaccgtctcctcag

SEQ ID NO: 230 - protein, 119 aa, Mus sp.
EVQLVESGGGLVQPKGSLKLSCAASGFAFNSYAMHWVCQAPGKGLEWVARIRIKSGNYATSYAGSVTDRFTVSRDDSQNLFYLQMNNLKTEDTAMYYCVREWEGAMDYWGQGTSVTVSS

SEQ ID NO: 231 - DNA, 24 bp, Mus sp.
ggtttcgccttcaattcctatgcc

SEQ ID NO: 232 - DNA, 30 bp, Mus sp.
ataagaattaaaagtggtaattatgcaaca

SEQ ID NO: 233 - DNA, 30 bp, Mus sp.
gtgagagagtgggaaggggctatggactac

SEQ ID NO: 234 - protein, 8 aa, Mus sp.
GFAFNSYA

SEQ ID NO: 235 - protein, 10 aa, Mus sp.
IRIKSGNYAT

SEQ ID NO: 236 - protein, 10 aa, Mus sp.
VREWEGAMDY

SEQ ID NO: 237 - DNA, 337 bp, Mus sp.
gatgttgtgatgacccagactcccctcactttgtcggttaccattggacagccagcctccatctcttgcacgtcaagtcagagcctcttaaatagtgatggggagacatatttgaattggttgttacagaggccaggccagtctccaaagcgcctaatccatctggtgtctaaactggactctggagtccctgacaggatcactggcagtggatctgggacagatttcacactgaaaatcaacagagtggaggctgaggatttgggaatttattattgctggcaaggtacacattttccgtggacgttcggtggaggcaccaagctggaaatcaaac

SEQ ID NO: 238 - protein, 112 aa, Mus sp.
DVVMTQTPLTLSVTIGQPASISCTSSQSLLNSDGETYLNWLLQRPGQSPKRLIHLVSKLDSGVPDRITGSGSGTDFTLKINRVEAEDLGIYYCWQGTHFPWTFGGGTKLEIK

SEQ ID NO: 239 - DNA, 33 bp, Mus sp.
cagagcctcttaaatagtgatggggagacatat

SEQ ID NO: 240 - DNA, 9 bp, Mus sp.
ctggtgtct

SEQ ID NO: 241 - DNA, 27 bp, Mus sp.
tggcaaggtacacattttccgtggacg

SEQ ID NO: 242 - protein, 11 aa, Mus sp.
QSLLNSDGETY

SEQ ID NO: 243 - protein, 3 aa, Mus sp.
LVS

SEQ ID NO: 244 - protein, 9 aa, Mus sp.
WQGTHFPWT

SEQ ID NO: 245 - DNA, 349 bp, Mus sp.
caggtccagttgcagcagtctggaactgagctggtaaggcctgggacttcagtgaagatatcctgcaaggcttctggatacgacttcactaacttttggctaggttggataaagcagaggcctggacatggacttgaatggattggagatatttaccctggaggtgataatacttactacaatgagaagttcaagggcaaagtcacgctgactgcagacaaatcctcgaacacagcctatatgcagttcagtagcctgacatctgaggactctgctgtctatttctgttttatgattctctatactttggactactggggtcaaggaacctcagtcaccgtctcctcag

SEQ ID NO: 246 - protein, 116 aa, Mus sp.
QVQLQQSGTELVRPGTSVKISCKASGYDFTNFWLGWIKQRPGHGLEWIGDIYPGGDNTYYNEKFKGKVTLTADKSSNTAYMQFSSLTSEDSAVYFCFMILYTLDYWGQGTSVTVSS

SEQ ID NO: 247 - DNA, 24 bp, Mus sp.
ggatacgacttcactaacttttgg

SEQ ID NO: 248 - DNA, 24 bp, Mus sp.
atttaccctggaggtgataatact

SEQ ID NO: 249 - DNA, 27 bp, Mus sp.
tttatgattctctatactttggactac

SEQ ID NO: 250 - protein, 8 aa, Mus sp.
GYDFTNFW

SEQ ID NO: 251 - protein, 8 aa, Mus sp.
IYPGGDNT

SEQ ID NO: 252 - protein, 9 aa, Mus sp.
FMILYTLDY

SEQ ID NO: 253 - DNA, 337 bp, Mus sp.
gatgttttgatgacccaaactccactctccctgcctgtcagtcttggagatcaagcctccatctcttgcagatctagtcagagcattgtacatagtaatggagacacctatttagaatggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggagtttattactgctttcaaggttcacattttccgtggacgttcggtggaggcaccaagctggaaatcaaac

SEQ ID NO: 254 - protein, 112 aa, Mus sp.
DVLMTQTPLSLPVSLGDQASISCRSSQSIVHSNGDTYLEWYLQKPGQSPKLLIYKVSNRFSGVPDRFSGSGSGTDFTLRISRVEAEDLGVYYCFQGSHFPWTFGGGTKLEIK

SEQ ID NO: 255 - DNA, 33 bp, Mus sp.
cagagcattgtacatagtaatggagacacctat

SEQ ID NO: 256 - DNA, 9 bp, Mus sp.
aaagtttcc

SEQ ID NO: 257 - DNA, 27 bp, Mus sp.
tttcaaggttcacattttccgtggacg

SEQ ID NO: 258 - protein, 11 aa, Mus sp.
QSIVHSNGDTY

SEQ ID NO: 259 - protein, 3 aa, Mus sp.
KVS

SEQ ID NO: 260 - protein, 9 aa, Mus sp.
FQGSHFPWT

SEQ ID NO: 261 - DNA, 358 bp, Mus sp.
gatgtgcagctggtggagtctgggggaggcttagtgcagcctggagggtcccggaaactctcctgtacagcctctggattcactttcagtagctttggaatgcactgggttcgtcaggctccagagaaggggctggagtgggtcgcatacattagtagtggcagtagtaaaatctactatgcagacacggtgaagggccgattcaccatctccagagacaatcccaagaacaccctgttcctgcaaatgaccagtctaaggtctgaggacacggccatgtattactgtgcaagagggtggtacgagggggcctggtttgcttactggggccaagggactctggtcactgtctctgcag

SEQ ID NO: 262 - protein, 119 aa, Mus sp.
DVQLVESGGGLVQPGGSRKLSCTASGFTFSSFGMHWVRQAPEKGLEWVAYISSGSSKIYYADTVKGRFTISRDNPKNTLFLQMTSLRSEDTAMYYCARGWYEGAWFAYWGQGTLVTVSA

SEQ ID NO: 263 - DNA, 24 bp, Mus sp.
ggattcactttcagtagctttgga

SEQ ID NO: 264 - DNA, 24 bp, Mus sp.
attagtagtggcagtagtaaaatc

SEQ ID NO: 265 - DNA, 36 bp, Mus sp.
gcaagagggtggtacgagggggcctggtttgcttac

SEQ ID NO: 266 - protein, 8 aa, Mus sp.
GFTFSSFG

SEQ ID NO: 267 - protein, 8 aa, Mus sp.
ISSGSSKI

SEQ ID NO: 268 - protein, 12 aa, Mus sp.
ARGWYEGAWFAY

SEQ ID NO: 269 - DNA, 334 bp, Mus sp.
gacattgtgctgacccaatctccagcttctttggctgtgtctctagggcagagggcctccatctcctgcaaggccagccaaagtgttgatcatgatggtgatagttatatgaactggtaccaacagaaaccaggacagccacccaaactcctcatctatgctacatccaatctagaatctgggatcccagccaggtttagtggcagtgggtctgggacagacttcaccctcaacatccatcctgtggaggaggaggatgctgcaacctattactgtcagcagagttatgaggttccgctcacgttcggtgctgggaccaagctggagctgaaac

SEQ ID NO: 270 - protein, 111 aa, Mus sp.
DIVLTQSPASLAVSLGQRASISCKASQSVDHDGDSYMNWYQQKPGQPPKLLIYATSNLESGIPARFSGSGSGTDFTLNIHPVEEEDAATYYCQQSYEVPLTFGAGTKLELK

SEQ ID NO: 271 - DNA, 30 bp, Mus sp.
caaagtgttgatcatgatggtgatagttat

SEQ ID NO: 272 - DNA, 9 bp, Mus sp.
gctacatcc

SEQ ID NO: 273 - DNA, 27 bp, Mus sp.
cagcagagttatgaggttccgctcacg

SEQ ID NO: 274 - protein, 10 aa, Mus sp.
QSVDHDGDSY

SEQ ID NO: 275 - protein, 3 aa, Mus sp.
ATS

SEQ ID NO: 276 - protein, 9 aa, Mus sp.
QQSYEVPLT

SEQ ID NO: 277 - DNA, 343 bp, Mus sp.
gaggtccagctgcaacagtctggacctgagctggtgaagcctggggcttcagtgaagatatcctgcaggacttctggatacacattcactgaatacaccattcactgggtgaagcagagccgtggaaagagccttgagtggattggaggtattaatcctaaccatggtggtactctctacaaccagaagttcaaggtcaaggccacattgactgtagacaagtcctccagcacagcctacatggagctccgcagcctgacatctgaggattctgcagtctattactgtgcaagatttacttacgactactggggccaaggcaccactctcacagtctcctcag

SEQ ID NO: 278 - protein, 114 aa, Mus sp.
EVQLQQSGPELVKPGASVKISCRTSGYTFTEYTIHWVKQSRGKSLEWIGGINPNHGGTLYNQKFKVKATLTVDKSSSTAYMELRSLTSEDSAVYYCARFTYDYWGQGTTLTVSS

SEQ ID NO: 279 - DNA, 24 bp, Mus sp.
ggatacacattcactgaatacacc

SEQ ID NO: 280 - DNA, 24 bp, Mus sp.
attaatcctaaccatggtggtact

SEQ ID NO: 281 - DNA, 21 bp, Mus sp.
gcaagatttacttacgactac

SEQ ID NO: 282 - protein, 8 aa, Mus sp.
GYTFTEYT

SEQ ID NO: 283 - protein, 8 aa, Mus sp.
INPNHGGT

SEQ ID NO: 284 - protein, 7 aa, Mus sp.
ARFTYDY

SEQ ID NO: 285 - DNA, 319 bp, Mus sp.
gaaaatgttctcacccagtctccagcaatcatgtctgcatctccaggggaaaaggtcaccatgacctgcagtgccagctcaagtgtaacttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccgtacacgttcggaggggggaccaagctggaaataaaac

SEQ ID NO: 286 - protein, 106 aa, Mus sp.
ENVLTQSPAIMSASPGEKVTMTCSASSSVTYMHWYQQKSSTSPKLWIYDTSKLASGVPGRFSGSGSGNSYSLTISSMEAEDVATYYCFQGSGYPYTFGGGTKLEIK

SEQ ID NO: 287 - DNA, 21 bp, Mus sp.
gccagctcaagtgtaacttac

SEQ ID NO: 288 - DNA, 9 bp, Mus sp.
gacacatcc

SEQ ID NO: 289 - DNA, 27 bp, Mus sp.
tttcaggggagtgggtacccgtacacg

SEQ ID NO: 290 - protein, 7 aa, Mus sp.
ASSSVTY

SEQ ID NO: 291 - protein, 3 aa, Mus sp.
DTS

SEQ ID NO: 292 - protein, 9 aa, Mus sp.
FQGSGYPYT

SEQ ID NO: 293 - DNA, 349 bp, Mus sp.
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggaaagccaacatatgttgatgacttcaagggacggtttgccttctctttggaaacctctgccaacactgcctatttgcagatcaacaacctcaaaaatgaggacacggctacatatttctgtgaaagtggaggttacgacgaggactactggggccaaggcaccactctcacagtctcctcag

SEQ ID NO: 294 - protein, 116 aa, Mus sp.
QIQLVQSGPELKKPGETVKISCKASGYTFTNYGMNWVKQAPGKGLKWMGWINTYTGKPTYVDDFKGRFAFSLETSANTAYLQINNLKNEDTATYFCESGGYDEDYWGQGTTLTVSS

SEQ ID NO: 295 - DNA, 24 bp, Mus sp.
gggtataccttcacaaactatgga

SEQ ID NO: 296 - DNA, 24 bp, Mus sp.
ataaacacctacactggaaagcca

SEQ ID NO: 297 - DNA, 27 bp, Mus sp.
gaaagtggaggttacgacgaggactac

SEQ ID NO: 298 - protein, 8 aa, Mus sp.
GYTFTNYG

SEQ ID NO: 299 - protein, 8 aa, Mus sp.
INTYTGKP

SEQ ID NO: 300 - protein, 9 aa, Mus sp.
ESGGYDEDY

SEQ ID NO: 301 - DNA, 337 bp, Mus sp.
gatgttttgatgacccaaactccactctccctgcctgtcagtcttggagatcaagcctccatctcttgcagatctagtcagaacattgtacatagtaatggaaacacctatttagaatggtacctgcagaaatcaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacattttccgtggacgttcggtggaggcaccaagctggaaatcaaac

SEQ ID NO: 302 - protein, 112 aa, Mus sp.
DVLMTQTPLSLPVSLGDQASISCRSSQNIVHSNGNTYLEWYLQKSGQSPKLLIYKVSNRFSGVPDRFSGSGSGTDFTLKISRVEAEDLGVYYCFQGSHFPWTFGGGTKLEIK

SEQ ID NO: 303 - DNA, 33 bp, Mus sp.
cagaacattgtacatagtaatggaaacacctat

SEQ ID NO: 304 - DNA, 9 bp, Mus sp.
aaagtttcc

SEQ ID NO: 305 - DNA, 27 bp, Mus sp.
tttcaaggttcacattttccgtggacg

SEQ ID NO: 306 - protein, 11 aa, Mus sp.
QNIVHSNGNTY

SEQ ID NO: 307 - protein, 3 aa, Mus sp.
KVS

SEQ ID NO: 308 - protein, 9 aa, Mus sp.
FQGSHFPWT

SEQ ID NO: 309 - DNA, 358 bp, Mus sp.
caggtgcagctgaaggagtcaggacctggcctggtggcgccctcacagagcctgtccatcacatgcactgtctcagggttctccttaaccgactatggtataacctggattcgccagcctccaggaaagggtctggagtggctgggagtaatatggggtggtggaagcgcatactataatttagttctcaaatccagactgagcatcagcaaggacaactccaagagtcaagttttcttaaaaatgaacagtctgcaaactgatgacacagccatgtactactgtgccaaacatcggactttgattacgactgctatggactactggggtcaaggaatttcagtcaccgtctcctcag

SEQ ID NO: 310 - protein, 119 aa, Mus sp.
QVQLKESGPGLVAPSQSLSITCTVSGFSLTDYGITWIRQPPGKGLEWLGVIWGGGSAYYNLVLKSRLSISKDNSKSQVFLKMNSLQTDDTAMYYCAKHRTLITTAMDYWGQGISVTVSS

SEQ ID NO: 311 - DNA, 24 bp, Mus sp.
gggttctccttaaccgactatggt

SEQ ID NO: 312 - DNA, 21 bp, Mus sp.
atatggggtggtggaagcgca

SEQ ID NO: 313 - DNA, 39 bp, Mus sp.
gccaaacatcggactttgattacgactgctatggactac

SEQ ID NO: 314 - protein, 8 aa, Mus sp.
GFSLTDYG

SEQ ID NO: 315 - protein, 7 aa, Mus sp.
IWGGGSA

SEQ ID NO: 316 - protein, 13 aa, Mus sp.
AKHRTLITTAMDY

SEQ ID NO: 317 - DNA, 337 bp, Mus sp.
gatgttttgatgacccaaactccactctccctgcctgtcagtcttggagatcaagcctccatctcttgcagatctagtcagaacattgtacatagtgatggaaacacctatttagaatggtacctgcagaaaccaggccagtctccaaagctcctgatctacaaattttccaaccgattttctggggtcccagacaggttcagtggcagtggatcagggacagatttcacactcaagatcagcagagtggaggctgaggatctgggagtttattactgctttcaaggttcacatgttcctcccacgttcggtgctgggaccaagctggagctgaaac

SEQ ID NO: 318 - protein, 112 aa, Mus sp.
DVLMTQTPLSLPVSLGDQASISCRSSQNIVHSDGNTYLEWYLQKPGQSPKLLIYKFSNRFSGVPDRFSGSGSGTDFTLKISRVEAEDLGVYYCFQGSHVPPTFGAGTKLELK

SEQ ID NO: 319 - DNA, 33 bp, Mus sp.
cagaacattgtacatagtgatggaaacacctat

SEQ ID NO: 320 - DNA, 9 bp, Mus sp.
aaattttcc

SEQ ID NO: 321 - DNA, 27 bp, Mus sp.
tttcaaggttcacatgttcctcccacg

SEQ ID NO: 322 - protein, 11 aa, Mus sp.
QNIVHSDGNTY

SEQ ID NO: 323 - protein, 3 aa, Mus sp.
KFS

SEQ ID NO: 324 - protein, 9 aa, Mus sp.
FQGSHVPPT

SEQ ID NO: 325 - DNA, 364 bp, Mus sp.
gaagtgaagctggtggagtctgggggaggcttagtgaagtctggagggtccctgaaactctcctgtgcagtctctggattcactttcagtacctatgccatgtcttgggttcgccagattccggagaagaggctggagtgggtcgcaaccattagtaatggtggtagttatatctactattcagacagtgtgaagggtcgattcaccatctccagagacaatgccaagaacaccctgtacctgcaaatgagcagtctgaggtctgaggacacggccatgtattactgtgcacgacatagggagtcctataggtacgactggtttgcttactggggccaagggactctggtcactgtctctgcag

SEQ ID NO: 326 - protein, 121 aa, Mus sp.
EVKLVESGGGLVKSGGSLKLSCAVSGFTFSTYAMSWVRQIPEKRLEWVATISNGGSYIYYSDSVKGRFTISRDNAKNTLYLQMSSLRSEDTAMYYCARHRESYRYDWFAYWGQGTLVTVSA

SEQ ID NO: 327 - DNA, 24 bp, Mus sp.
ggattcactttcagtacctatgcc

SEQ ID NO: 328 - DNA, 24 bp, Mus sp.
attagtaatggtggtagttatatc

SEQ ID NO: 329 - DNA, 42 bp, Mus sp.
gcacgacatagggagtcctataggtacgactggtttgcttac

SEQ ID NO: 330 - protein, 8 aa, Mus sp.
GFTFSTYA

SEQ ID NO: 331 - protein, 8 aa, Mus sp.
ISNGGSYI

SEQ ID NO: 332 - protein, 14 aa, Mus sp.
ARHRESYRYDWFAY

SEQ ID NO: 333 - protein, 26 aa, Artificial Sequence
  huCAN30G5 Chain K, FR1
DIVLTQSPLSLPVTLGQPASISCRSS

SEQ ID NO: 334 - protein, 17 aa, Artificial Sequence
  huCAN30G5 Chain K, FR2
LHWYQQRPGQSPRLLIY

SEQ ID NO: 335 - protein, 36 aa, Artificial Sequence
  huCAN30G5 Chain K, FR3
NRFSGVPDRFSGSGSGTDFTLKISRVEAEDVGVYYC

SEQ ID NO: 336 - protein, 10 aa, Artificial Sequence
  huCAN30G5 Chain K, FR4
FGGGTKVEIK

SEQ ID NO: 337 - protein, 25 aa, Artificial Sequence
  huCAN30G5 Chain H, FR1
EVQLVESGGGLVQPGGSLKLSCAAS

SEQ ID NO: 338 - protein, 17 aa, Artificial Sequence
  huCAN30G5 Chain H, FR2
MHWVRQASGKGLEWVAR

SEQ ID NO: 339 - protein, 38 aa, Artificial Sequence
  huCAN30G5 Chain H, FR3
SYAGSVKGRFTVSRDDSKNTFYLQMNSLKTEDTAMYYC

SEQ ID NO: 340 - protein, 11 aa, Artificial Sequence
  huCAN30G5 Chain H, FR4
WGQGTLVTVSS

SEQ ID NO: 341 - protein, 26 aa, Artificial Sequence
  rehuCAN30G5 Chain K, FR1
DIVLTQSPLSLPVTLGQPASISCRSS

SEQ ID NO: 342 - protein, 17 aa, Artificial Sequence
  rehuCAN30G5 Chain K, FR2
LHWYLQKPGQSPRLLIY

SEQ ID NO: 343 - protein, 36 aa, Artificial Sequence
  rehuCAN30G5 Chain K, FR3
NRFSGVPDRFSGSGSGTDFTLKISRVEAEDVGVYFC

SEQ ID NO: 344 - protein, 10 aa, Artificial Sequence
  rehuCAN30G5 Chain K, FR4
FGGGTKLEIK

SEQ ID NO: 345 - protein, 25 aa, Artificial Sequence
  rehuCAN30G5 Chain H, FR1
EVQLVESGGGLVQPGGSLKLSCAAS

SEQ ID NO: 346 - protein, 17 aa, Artificial Sequence
  rehuCAN30G5 Chain H, FR2
MHWVCQASGKGLEWVAR

SEQ ID NO: 347 - protein, 38 aa, Artificial Sequence
  rehuCAN30G5 Chain H, FR3
SYAGSVKGRFTVSRDDSKSLFYLQMNNLKTEDTAMYYC

SEQ ID NO: 348 - protein, 11 aa, Artificial Sequence
  rehuCAN30G5 Chain H, FR4
WGQGTLVTVSS

SEQ ID NO: 349 - protein, 26 aa, Artificial Sequence
  cdrCAN30G5 Chain K, FR1
DVVMTQSPLSLPVTLGQPASISCRSS

SEQ ID NO: 350 - protein, 17 aa, Artificial Sequence
  cdrCAN30G5 Chain K, FR2
LNWFQQRPGQSPRRLIY

SEQ ID NO: 351 - protein, 36 aa, Artificial Sequence
  cdrCAN30G5 Chain K, FR3
NRDSGVPDRFSGSGSGTDFTLKISRVEAEDVGVYYC

SEQ ID NO: 352 - protein, 10 aa, Artificial Sequence
  cdrCAN30G5 Chain K, FR4
FGGGTKVEIK

SEQ ID NO: 353 - protein, 25 aa, Artificial Sequence
  cdrCAN30G5 Chain H, FR1
EVQLVESGGGLVQPGGSLKLSCAAS

SEQ ID NO: 354 - protein, 17 aa, Artificial Sequence
  cdrCAN30G5 Chain H, FR2
MHWVRQASGKGLEWVGR

SEQ ID NO: 355 - protein, 38 aa, Artificial Sequence
  cdrCAN30G5 Chain H, FR3
AYAASVKGRFTISRDDSKNTAYLQMNSLKTEDTAVYYC

SEQ ID NO: 356 - protein, 11 aa, Artificial Sequence
  cdrCAN30G5 Chain H, FR4
WGQGTLVTVSS

SEQ ID NO: 357 - protein, 26 aa, Artificial Sequence
  huCAN40G1 Chain K, FR1
DIVLTQSPASLAVSLGERATISCKAS

SEQ ID NO: 358 - protein, 17 aa, Artificial Sequence
  huCAN40G1 Chain K, FR2
MNWYQQKPGQPPKLLIY

SEQ ID NO: 359 - protein, 36 aa, Artificial Sequence
  huCAN40G1 Chain K, FR3
NLESGIPARFSGSGSGTDFTLTISSLQAEDVATYYC

SEQ ID NO: 360 - protein, 10 aa, Artificial Sequence
  huCAN40G1 Chain K, FR4
FGAGTKLEIK

SEQ ID NO: 361 - protein, 25 aa, Artificial Sequence
  huCAN40G1 Chain H, FR1
EVQLQQSGPEVKKPGASVKVSCRTS

SEQ ID NO: 362 - protein, 17 aa, Artificial Sequence
  huCAN40G1 Chain H, FR2
IHWVKQAPGKGLEWIGG

SEQ ID NO: 363 - protein, 38 aa, Artificial Sequence
  huCAN40G1 Chain H, FR3
LYNQKFKGRVTLTVDKSSSTAYMELSRLRSDDTAVYYC

SEQ ID NO: 364 - protein, 11 aa, Artificial Sequence
  huCAN40G1 Chain H, FR4
WGQGTLVTVSS

SEQ ID NO: 365 - protein, 26 aa, Artificial Sequence
  rehuCAN40G1 Chain K, FR1
DIVLTQSPASLAVSLGERATISCKAS

SEQ ID NO: 366 - protein, 17 aa, Artificial Sequence
  rehuCAN40G1 Chain K, FR2
MNWYQQKPGQPPKLLIY

SEQ ID NO: 367 - protein, 36 aa, Artificial Sequence
  rehuCAN40G1 Chain K, FR3
NLESGIPARFSGSGSGTDFTLNISSVQAEDVATYYC

SEQ ID NO: 368 - protein, 10 aa, Artificial Sequence
  rehuCAN40G1 Chain K, FR4
FGAGTKLELK

SEQ ID NO: 369 - protein, 25 aa, Artificial Sequence
  rehuCAN40G1 Chain H, FR1
EVQLQQSGPEVVKPGASVKISCRTS

SEQ ID NO: 370 - protein, 17 aa, Artificial Sequence
  rehuCAN40G1 Chain H, FR2
IHWVKQAPGKGLEWIGG

SEQ ID NO: 371 - protein, 38 aa, Artificial Sequence
  rehuCAN40G1 Chain H, FR3
LYNQKFKGRATLTVDKSSSTAYMELSRLRSDDTAVYYC

SEQ ID NO: 372 - protein, 11 aa, Artificial Sequence
  rehuCAN40G1 Chain H, FR4
WGQGTLLTVSS

SEQ ID NO: 373 - protein, 26 aa, Artificial Sequence
  cdrCAN40G1 Chain K, FR1
DIVMTQSPDSLAVSLGERATINCKSS

SEQ ID NO: 374 - protein, 17 aa, Artificial Sequence
  cdrCAN40G1 Chain K, FR2
LAWYQQKPGQPPKLLIY

SEQ ID NO: 375 - protein, 36 aa, Artificial Sequence
  cdrCAN40G1 Chain K, FR3
TRESGVPDRFSGSGSGTDFTLTISSLQAEDVAVYYC

SEQ ID NO: 376 - protein, 10 aa, Artificial Sequence
  cdrCAN40G1 Chain K, FR4
FGQGTKLEIK

SEQ ID NO: 377 - protein, 25 aa, Artificial Sequence [sequence omitted]

SEQ ID NO: 378 - protein, 17 aa, Artificial Sequence
  cdrCAN40G1 Chain H, FR2
MHWVRQAPGQGLEWMGW

SEQ ID NO: 379 - protein, 38 aa, Artificial Sequence
  cdrCAN40G1 Chain H, FR3
NYAQKFQGRVTMTRDTSISTAYMELSRLRSDDTAVYYC

SEQ ID NO: 380 - protein, 11 aa, Artificial Sequence
  cdrCAN40G1 Chain H, FR4
WGQGTLVTVSS

SEQ ID NO: 381 - protein, 26 aa, Artificial Sequence
  huCAN54G2 Chain K, FR1
DVVMTQTPLTLPVTLGQPASISCTSS

SEQ ID NO: 382 - protein, 17 aa, Artificial Sequence
  huCAN54G2 Chain K, FR2
LNWLLQRPGQSPKRLIH

SEQ ID NO: 383 - protein, 36 aa, Artificial Sequence
  huCAN54G2 Chain K, FR3
KLDSGVPDRFSGSGSGTDFTLKISRVEAEDVGVYYC

SEQ ID NO: 384 - protein, 10 aa, Artificial Sequence
  huCAN54G2 Chain K, FR4
FGGGTKVEIK

SEQ ID NO: 385 - protein, 25 aa, Artificial Sequence
  huCAN54G2 Chain H, FR1
QVQLQQSGTEVKKPGASVKVSCKAS

SEQ ID NO: 386 - protein, 17 aa, Artificial Sequence
  huCAN54G2 Chain H, FR2
LGWIRQAPGQGLEWIGD

SEQ ID NO: 387 - protein, 38 aa, Artificial Sequence
  huCAN54G2 Chain H, FR3
YYNEKFKGRVTLTADKSSNTAYMELSSLRSEDTAVYFC

SEQ ID NO: 388 - protein, 11 aa, Artificial Sequence
  huCAN54G2 Chain H, FR4
WGQGTLVTVSS

SEQ ID NO: 389 - protein, 26 aa, Artificial Sequence
  rehuCAN54G2 Chain K, FR1
DVVMTQTPLTLPVTLGQPASISCTSS

SEQ ID NO: 390 - protein, 17 aa, Artificial Sequence
  rehuCAN54G2 Chain K, FR2
LNWLLQRPGQSPKRLIH

SEQ ID NO: 391 - protein, 36 aa, Artificial Sequence
  rehuCAN54G2 Chain K, FR3
KLDSGVPDRISGSGSGTDFTLKISRVEAEDLGIYYC

SEQ ID NO: 392 - protein, 10 aa, Artificial Sequence
  rehuCAN54G2 Chain K, FR4
FGGGTKVEIK

SEQ ID NO: 393 - protein, 25 aa, Artificial Sequence
  rehuCAN54G2 Chain H, FR1
QVQLQQSGTEVVKPGASVKISCKAS

SEQ ID NO: 394 - protein, 17 aa, Artificial Sequence
  rehuCAN54G2 Chain H, FR2
LGWIKQAPGQGLEWIGD

SEQ ID NO: 395 - protein, 38 aa, Artificial Sequence
  rehuCAN54G2 Chain H, FR3
YYNEKFKGKVTLTADKSSNTAYMEFSSLRSEDTAVYFC

SEQ ID NO: 396 - protein, 11 aa, Artificial Sequence
  rehuCAN54G2 Chain H, FR4
WGQGTLVTVSS

SEQ ID NO: 397 - protein, 26 aa, Artificial Sequence
  cdrCAN54G2 Chain K, FR1
DVVMTQSPLSLPVTLGQPASISCRSS

SEQ ID NO: 398 - protein, 17 aa, Artificial Sequence
  cdrCAN54G2 Chain K, FR2
LNWFQQRPGQSPRRLIY

SEQ ID NO: 399 - protein, 36 aa, Artificial Sequence; identical to SEQ ID NO: 351
  cdrCAN54G2 Chain K, FR3

SEQ ID NO: 400 - protein, 10 aa, Artificial Sequence
  cdrCAN54G2 Chain K, FR4
FGGGTKVEIK

SEQ ID NO: 401 - protein, 25 aa, Artificial Sequence [sequence omitted]

SEQ ID NO: 402 - protein, 17 aa, Artificial Sequence
  cdrCAN54G2 Chain H, FR2
MHWVRQAPGQGLEWMGI

SEQ ID NO: 403 - protein, 38 aa, Artificial Sequence
  cdrCAN54G2 Chain H, FR3
SYAQKFQGRVTMTRDTSTSTVYMELSSLRSEDTAVYYC

SEQ ID NO: 404 - protein, 11 aa, Artificial Sequence
  cdrCAN54G2 Chain H, FR4
WGQGTLVTVSS